(19) United States
(12) Patent Application Publication
(10) Pub. No.: US 2020/0188388 A1
(43) Pub. Date: Jun. 18, 2020

(54) METHODS AND COMPOSITIONS FOR POTENTIATING CNS DRUGS AND REDUCING THEIR SIDE EFFECTS

(71) Applicant: RVX THERAPEUTICS LTD., Kiriat Yam (IL)

(72) Inventors: Sergey SERDYUK, Jerusalem (IL); Vladimir RITTER, Kiriat Yam (IL)

(21) Appl. No.: 16/474,237

(22) PCT Filed: Dec. 27, 2017
(86) PCT No.: PCT/IL2017/051391

§ 371 Date: Jun. 27, 2019

Related U.S. Application Data
(60) Provisional application No. 62/439,905, filed on Dec. 29, 2016.
(60) Provisional application No. 62/439,901, filed on Dec. 29, 2016.

(51) Int. Cl. A61K 31/485; A61K 31/4152; A61K 31/165; A61K 31/13; A61P 25/16

(52) U.S. Cl. CPC A61K 31/485; A61K 31/4152; A61P 25/16; A61K 31/13; A61K 31/165

(57) ABSTRACT

The present invention provides safe, low-dose, therapeutic combinations of CNS drugs with peripheral adrenergic receptor agonists, and their use in methods for treating various conditions.

### FIELD OF THE INVENTION

The present invention provides combinations of drugs which affect the central nervous system (CNS) and peripheral adrenergic receptor agonists which potentiate and reduce the side effects of these drugs. The present invention further provides methods for the use of such combinations in therapy.

### BACKGROUND OF THE INVENTION

Patients receiving drugs that affect the CNS are usually treated with the lowest effective dose of these drugs, under the assumption that the use of low dosages should minimize or eliminate drug-associated side effects. The main disadvantage of this rationale is the inability to attain the maximal therapeutic effect. Under these conditions, the efficacy of CNS drugs in the treatment of acute or chronic forms of CNS disorders is limited.

Previous attempts at bolstering the therapeutic action of CNS drugs, especially in the generally-accepted high therapeutic doses, to achieve the maximal therapeutic effect by using combinations of different CNS drugs usually resulted in increased, rather than decreased, side effects and therefore may not be safe.

U.S. Pat. No. 6,833,377 and EP Patent 1359939 to one of the present inventors disclose a method of potentiating the activity of a CNS drug comprising systemically administrating said drug together with an effective amount of a compound which affects peripheral chemoreceptors and, optionally, with an effective amount of a stimulator of osmoreceptors.

U.S. Pat. No. 8,729,070 is directed to CNS pharmaceutical compositions comprising a CNS active agent and preferably at least two vagal neuromodulators, one of which is a mechanoreceptor stimulator. U.S. Pat. No. 8,729,070 further discloses methods of reducing CNS active agent side-effects.

There remains a need in the field of CNS therapy for safe, low-dose and highly-effective compositions of CNS drugs.

### SUMMARY OF THE INVENTION

The present invention relates to methods of potentiating the activity and/or reducing the side effects of CNS drugs by their co-administration together with peripheral adrenergic receptor agonists. The present invention is based in part on the unexpected ability of certain combinations of CNS drugs and peripheral adrenergic receptor agonists to potentiate the therapeutic efficacy of CNS drugs, as well as to reduce the side effects of CNS drugs in both standard (known as therapeutic) and sub-standard (known as non-therapeutic) doses.

The ability of combinations of CNS drugs and peripheral adrenergic receptor agonists to potentiate CNS drugs safely can be utilized to administer dosages of CNS drugs and peripheral adrenergic receptor agonists which up till now were considered to be sub-standard (or non-therapeutic), without the risk of diminishing the therapeutic efficacy of the CNS drugs or developing drug-related side effects, as demonstrated herein. This ability can further be utilized to administer conventional dosages of combinations of CNS drugs together with peripheral adrenergic receptor agonists thereby gaining an increased therapeutic efficacy without development of related side effects, as also demonstrated herein.

As exemplified below, the combined administration of anti-parkinsonian drugs or analgesics together with peripheral adrenergic receptor agonists and optionally with NMDA receptor antagonists in sub-standard doses (FDA List of Approved Drug Products, 37^{th} Edition, 2017) results in a safe anti-parkinsonian or analgesic effect, which is at least equal to the anti-parkinsonian or analgesic effect of these drugs alone in standard therapeutic doses, without the development of side effects, which are routinely invoked while using standard, high doses of such drugs.

As further exemplified below, the combined administration of different drugs in standard doses results in a safe and synergistic anti-parkinsonian or analgesic effect, surpassing the anti-parkinsonian and analgesic effect of these drugs alone in standard therapeutic doses, and reduces side effects of standard doses of analgesics and anti-parkinsonian drugs.

The present invention provides, in one aspect, a method of potentiating the activity or reducing at least one side effect of a CNS drug selected from the group consisting of an anti-parkinsonian drug and an analgesic drug in a subject in need thereof, comprising systemically administering to the subject (a) at least one first CNS drug, selected from the group consisting of an anti-parkinsonian drug and an analgesic drug; (b) at least one second CNS drug, which is an NMDA receptor antagonist; and (c) at least one peripheral adrenergic receptor agonist.

In certain embodiments, the first CNS drug is an anti-parkinsonian drug. In certain embodiments, the first CNS drug is an analgesic drug. In some embodiments the drugs are each administered in separate dosage forms. In alternative embodiments the drugs may be in a single dosage form. In yet further embodiments two of the drugs may be combined in a single dosage form and the third may be administered in a separate dosage form.

In certain embodiments, the anti-parkinsonian drug is selected from the group consisting of (a) L-3,4-dihydroxyphenylalanine (levodopa); (b) a dopamine agonist selected from the group consisting of bromocriptine, cabergoline, pergolide, pramipexole, ropinirole, piribedil, apomorphine, rigotine, quinagolide, fenoldopam, and lisuride; and (c) a monoamine oxidase B (MAOB) inhibitor selected from the group consisting of selegiline, desmethylselegiline, pargyline, rasagiline, lazabemide, milacemide, mofegiline, D-deprenyl and ladostigil. In particular embodiments, the anti-parkinsonian drug is levodopa.

In certain embodiments, the analgesic drug is selected from the group consisting of morphine, amitriptyline, dipyrone, fentanyl, promedolum, omnoponum, oxycodone, hydrocodone, hydromorphone, hydrocodone bitartrate, and buprenorphine. In certain embodiments, the analgesic drug is morphine, amitriptyline or dipyrone. In certain embodiments, the analgesic drug is morphine. In certain embodiments, the analgesic drug is amitriptyline. In certain embodiments, the analgesic drug is dipyrone.

In certain embodiments, the NMDA receptor antagonist is selected from the group of memantine, amantadine, dextromethorphan and ketamine. In certain embodiments, the NMDA receptor antagonist is memantine.

In certain embodiments, the peripheral adrenergic receptor agonist is selected from the group consisting of phenylephrine, epinephrine, midodrine and pseudoephedrine. In certain embodiments, the peripheral adrenergic receptor agonist is phenylephrine or epinephrine. In certain embodiments, the peripheral adrenergic receptor agonist is phenylephrine. In certain embodiments, the peripheral adrenergic receptor agonist is epinephrine.

In certain embodiments, the first CNS drug is selected from the group consisting of levodopa, morphine, amitriptyline or dipyrone; the NMDA receptor antagonist is memantine; and the peripheral adrenergic receptor agonist is selected from phenylephrine or epinephrine.

In certain embodiments, the first CNS drug is levodopa, the NMDA receptor antagonist is memantine, and the peripheral adrenergic receptor agonist is phenylephrine. In certain embodiments, the first CNS drug is morphine, the NMDA receptor antagonist is memantine, and the peripheral adrenergic receptor agonist is epinephrine. In certain embodiments, the first CNS drug is morphine, the NMDA receptor antagonist is memantine, and the peripheral adrenergic receptor agonist is phenylephrine. In certain embodiments, the first CNS drug is amitriptyline, the NMDA receptor antagonist is memantine, and the peripheral adrenergic receptor agonist is phenylephrine. In certain embodiments, the first CNS drug is dipyrone, the NMDA receptor antagonist is memantine, and the peripheral adrenergic receptor agonist is phenylephrine.

In certain embodiments, the side effect is selected from the group consisting hyperkinesia, sedation, hyperalgesia, catalepsy, dyskinesia, and addiction. In certain embodiments, the side effect is hyperkinesia. In certain embodiments, the side effect is sedation. In certain embodiments, the side effect is hyperalgesia. In certain embodiments, the side effect is catalepsy. In certain embodiments, the side effect is dyskinesia. In certain embodiments, the side effect is addiction.

In certain embodiments, the method comprises systemically administering to the subject 5-200 mg levodopa. In certain embodiments, the method comprises systemically administering to the subject 5, 10, 30, 50, 100 or 200 mg levodopa.

In certain embodiments, the method comprises systemically administering to the subject 0.1-50 mg morphine. In certain embodiments, the method comprises systemically administering to the subject 0.1, 0.2, 0.5, 1, 2, 4, 5, 10, 25, 30 or 50 mg morphine.

In certain embodiments, the method comprises systemically administering to the subject 0.5-20 mg amitriptyline. In certain embodiments, the method comprises systemically administering to the subject 0.5, 1 or 20 mg amitriptyline.

In certain embodiments, the method comprises systemically administering to the subject 0.1-40 mg dipyrone. In certain embodiments, the method comprises systemically administering to the subject 0.5, 1 or 40 mg dipyrone.

In certain embodiments, the method comprises systemically administering to the subject 5-30 mg memantine. In certain embodiments, the method comprises systemically administering to the subject 5, 10 or 30 mg memantine.

In certain embodiments, the method comprises systemically administering to the subject 0.1-3 mg phenylephrine. In certain embodiments, the method comprises systemically administering to the subject 0.1, 0.2, 0.3, 0.5, 1 or 3 mg phenylephrine.

In certain embodiments, the method comprises systemically administering to the subject 0.05-0.1 mg epinephrine. In certain embodiments, the method comprises systemically administering to the subject 0.05 or 0.1 mg epinephrine.

In certain embodiments, the method comprises systemically administering to the subject (a) 5-200, 5, 10, 30, 50, 100 or 200 mg levodopa, (b) 0.1-50, 0.1, 0.2, 0.5, 1, 2, 4, 5, 10, 25, 30 or 50 mg morphine (c) 0.5-20, 0.5, 1 or 20 mg amitriptyline or (d) 0.5-40, 0.5, 1 or 40 mg dipyrone; (e) 5-30, 5, 10 or 30 mg memantine; and (f) 0.1-3, 0.1, 0.2, 0.3, 0.5, 1 or 3 mg phenylephrine or (g) 0.05-0.1, 0.05 or 0.1 mg epinephrine.

In certain embodiments, the method comprises systemically administering to the subject the first CNS drug, the second CNS drug and the peripheral adrenergic receptor agonist in a molar ratio of 0.2-1000:1-300:1, respectively.

The present invention further provides, in another aspect, a method of treating Parkinson Disease (PD) or Progressive Supranuclear Palsy (PSP) or Parkinsonism syndrome in a subject in need, comprising systemically administering to the subject (a) at least one first CNS drug, which is an anti-parkinsonian drug; (b) at least one second CNS drug, which is an NMDA receptor antagonist; and (c) at least one peripheral adrenergic receptor agonist.

The present invention further provides, in another aspect, a method of treating pain or hyperalgesia in a subject in need, comprising systemically administering to the subject (a) at least one first CNS drug, which is an analgesic drug; (b) at least one second CNS drug, which is an NMDA receptor antagonist; and (c) at least one peripheral adrenergic receptor agonist.

The present invention further provides, in another aspect, a pharmaceutical composition, comprising (a) at least one first CNS drug, selected from the group consisting of an anti-parkinsonian drug and an analgesic drug; (b) at least one second CNS drug, which is an NMDA receptor antagonist; and (c) at least one peripheral adrenergic receptor agonist.

In certain embodiments, the pharmaceutical composition described above is for use in a method of potentiating the activity or reducing at least one side effect of first the CNS drug. In certain embodiments, the first CNS drug is an anti-parkinsonian drug, for use in a method of treating Parkinson Disease (PD) or Progressive Supranuclear Palsy (PSP) or Parkinsonism syndrome. In certain embodiments, the first CNS drug is an analgesic drug, for use in a method of treating pain or hyperalgesia.

The present invention further provides, in another aspect, a unit dosage form, comprising a pharmaceutical composition as described above.

The present invention provides, in another aspect, a combination of (a) at least one first CNS drug, selected from the group consisting of an anti-parkinsonian drug and an analgesic drug; (b) at least one second CNS drug, which is an NMDA receptor antagonist; and (c) at least one peripheral adrenergic receptor agonist, for use in a method of (i) potentiating the activity or reducing at least one side effect of the first CNS drug, (ii) treating Parkinson Disease (PD) or Progressive Supranuclear Palsy (PSP) or Parkinsonism syndrome, or (iii) treating pain or hyperalgesia, the method comprising systemic administration of the combination.

The present invention further provides, in another aspect, a method of potentiating the activity or reducing at least one side effect of a CNS drug selected from the group consisting of an anti-parkinsonian drug and an analgesic drug in a subject in need, comprising systemically administering to the subject (a) at least one CNS drug, wherein the CNS drug is levodopa; and (b) at least one peripheral adrenergic receptor agonist, wherein the peripheral adrenergic receptor agonist is phenylephrine or epinephrine; wherein the molar ratio of the CNS drug and peripheral adrenergic receptor agonist is in the range of 1-2000:1, respectively.

In certain embodiments, the CNS drug is levodopa and the peripheral adrenergic receptor agonist is phenylephrine or epinephrine. Each possibility represents a separate embodiment of the present invention. In certain embodiments, the CNS drug is levodopa and the peripheral adrenergic receptor agonist is phenylephrine. In certain embodiments, the CNS drug is levodopa and the peripheral adrenergic receptor agonist is epinephrine.

In certain embodiments, the side effect is selected from the group consisting hyperkinesia, sedation, hyperalgesia, catalepsy, dyskinesia, and addiction. In certain embodiments, the side effect is hyperkinesia. In certain embodiments, the side effect is sedation. In certain embodiments, the side effect is hyperalgesia. In certain embodiments, the side effect is catalepsy. In certain embodiments, the side effect is dyskinesia. In certain embodiments, the side effect is addiction.

In certain embodiments, the method comprises systemically administering to the subject 15-200 mg levodopa. In certain embodiments, the method comprises systemically administering to the subject 15, 30, 100 or 200 mg levodopa.

In certain embodiments, the peripheral adrenergic receptor agonist is phenylephrine. In certain embodiments, the method comprises systemically administering to the subject 0.2-3 mg phenylephrine. In certain embodiments, the method comprises systemically administering to the subject 0.2, 1 or 3 mg phenylephrine.

In certain embodiments, the peripheral adrenergic receptor agonist is epinephrine. In certain embodiments, the method comprises systemically administering to the subject 0.1-0.3 mg epinephrine. In certain embodiments, the method comprises systemically administering to the subject 0.1, 0.2 or 0.3 mg epinephrine.

In certain embodiments, the method comprises systemically administering to the subject (a) 15-200, 15, 30, 100 or 200 mg levodopa; and (b) 0.2-3, 0.2, 1 or 3 mg phenylephrine or (c) 0.1-0.3, 0.1, 0.2 or 0.3 mg epinephrine.

The present invention further provides, in another aspect, a method of treating Parkinson Disease (PD) or Progressive Supranuclear Palsy (PSP) or Parkinsonism syndrome in a subject in need, comprising systemically administering to the subject (a) at least one CNS drug, wherein the CNS drug is levodopa; and (b) at least one peripheral adrenergic receptor agonist, wherein the peripheral adrenergic receptor agonist is phenylephrine or epinephrine; wherein the molar ratio of the CNS drug and peripheral adrenergic receptor agonist is in the range of 1-2000:1, respectively.

The present invention further provides, in another aspect, a pharmaceutical composition, comprising (a) at least one CNS drug, wherein the CNS drug is levodopa; and (b) at least one peripheral adrenergic receptor agonist, wherein the peripheral adrenergic receptor agonist is phenylephrine or epinephrine; wherein the molar ratio of the CNS drug and the peripheral adrenergic receptor agonist is in the range of 1-2000:1, respectively.

The present invention further provides, in another aspect, a unit dosage form, comprising a pharmaceutical composition as described above.

The present invention further provides, in another aspect, a combination of (a) at least one CNS drug, wherein the CNS drug is levodopa; and (b) at least one peripheral adrenergic receptor agonist, wherein the peripheral adrenergic receptor agonist is phenylephrine or epinephrine; wherein the molar ratio of the CNS drug and peripheral adrenergic receptor agonist is in the range of 1-2000:1, respectively, for use in a method of (i) potentiating the activity or reducing at least one side effect of the CNS drug, or (ii) treating Parkinson Disease (PD) or Progressive Supranuclear Palsy (PSP) or Parkinsonism syndrome, the method comprising systemic administration of the combination.

It should be emphasized that embodiments relating to molar ratio ranges, specific molar ratios, weight ranges and specific weights of CNS drugs and/or peripheral adrenergic receptor agonists relate to pharmaceutical compositions in which the CNS drugs and the peripheral adrenergic receptor agonists may be combined. It is further explicitly understood that the drugs may be present in separate dosage forms. It is further explicitly understood that in methods of treatment, the CNS drugs and the peripheral adrenergic receptor agonists may be administered separately or together. It is further explicitly understood that when the drugs are administered in separate dosage forms they are administered substantially at the same time or in substantially overlapping schedules. Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention provides pharmaceutical compositions comprising synergistic combinations of CNS drugs and peripheral adrenergic receptor agonists, which provide therapeutic advantages over standard CNS drugs currently used for treating a variety of human diseases or disorders. Two major advantages of the pharmaceutical compositions of the present invention are the ability to efficiently treat subjects with lower-than-standard doses of CNS drugs without compromising therapeutical outcome, and the ability to treat subjects with standard doses of CNS drugs with superior therapeutical outcome while reducing CNS drug-related side effects which are routinely invoked while using CNS drugs in standard doses.

The present invention further provides novel methods for treating subjects receiving or in need of receiving CNS drug therapy. One such method comprises the administration of combinations of lower-than-standard doses of CNS drugs together with lower-than-standard doses of peripheral adrenergic receptor agonists. Another such method comprises the administration of combinations of lower-than-standard doses of CNS drugs together with standard doses of peripheral adrenergic receptor agonists. Such methods provide the therapeutic benefits of standard doses of CNS drugs, without the risk of common CNS drug-related side effects or CNS drug tolerability. Another such method comprises the administration of combinations of standard doses of CNS drugs together with standard doses of peripheral adrenergic receptor agonists. Such method potentiates the therapeutic efficacy and reduces the side effects of standard doses of CNS drugs. This method may be used for the safe treatment of drug-resistant subjects and/or for the treatment of acute forms of CNS diseases and disorders.

CNS drugs are approved for human therapy in a limited dosage range, taking into account the minimal dosage required to achieve a therapeutically-significant effect, the maximal dosage which would not invoke detrimental side-effects, and patient-specific indexes, such as weight and age. For example, levodopa and morphine have an approved therapeutic range in humans of 50-400 mg and 5-20 mg, respectively. Amitriptyline has an approved therapeutic range in humans of 25-100 mg, and dipyrone has an approved therapeutic range in humans of 0.2-1.0 mg. Memantine, for example, has an approved therapeutic range in humans of 5-20 mg. Similar to CNS drugs, peripheral adrenergic receptor agonists are also approved for human therapy in a limited dosage range. For example, phenylephrine and epinephrine have an approved therapeutic range in humans of 5-20 mg and 0.2-1 mg, respectively. According to the principles of the present invention, there are provided pharmaceutical compositions and methods of therapy which comprise or utilize combinations of CNS drugs and peripheral adrenergic receptor agonists in dosages below the known, approved therapeutic ranges, and/or minimize the side-effects of the approved therapeutic ranges.

Further according to the principles of the present invention, at least one drug is administered in a low dose, which is 10 fold to 100 fold lower than its standard dose, as described above, or administered in a sub-standard dose, which is 2 fold to 5 fold lower than its standard dose, as described above. For example, epinephrine having an approved therapeutic range in humans of 0.2 mg to 1 mg, may be administered at a dose of 0.05 mg to 0.1 mg, which is considered as a low dose (20 fold, 1 mg vs. 0.05 mg) or a sub-standard dose (2 fold, 0.2 mg vs. 0.1 mg).

Without being bound by any theory or mechanism, it is hypothesized that the safe potentiation of anti-parkinsonian drugs and analgesics is realized by the following two-stage mechanism: in the first stage, peripheral adrenergic receptor agonists reduce the effective dose of NMDA receptor antagonists, without development of side effects, and at the second stage these reduced effective doses of NMDA receptor antagonists potentiate the efficacy, decrease the effective doses and eliminate the side effects of anti-parkinsonian drugs and opioid and non-op ioid analgesics.

The present invention provides, in one aspect, a method of potentiating the activity or reducing at least one side effect of a CNS drug selected from the group consisting of an anti-parkinsonian drug and an analgesic drug in a subject in need, comprising systemically administering to the subject (a) at least one first CNS drug selected from the group consisting of an anti-parkinsonian drug and an analgesic drug; (b) at least one second CNS drug which is an NMDA receptor antagonist; and (c) at least one peripheral adrenergic receptor agonist.

The present invention further provides, in another aspect, a method of treating Parkinson Disease (PD) or Progressive Supranuclear Palsy (PSP) in a subject in need, comprising systemically administering to the subject (a) at least one first CNS drug which is an anti-parkinsonian drug; (b) at least one second CNS drug which is an NMDA receptor antagonist; and (c) at least one peripheral adrenergic receptor agonist.

The present invention further provides, in another aspect, a method of treating pain or hyperalgesia in a subject in need, comprising systemically administering to the subject (a) at least one first CNS drug which is an analgesic drug; (b) at least one second CNS drug which is an NMDA receptor antagonist; and (c) at least one peripheral adrenergic receptor agonist.

The present invention further provides, in another aspect, a pharmaceutical composition, comprising (a) at least one first CNS drug selected from the group consisting of an anti-parkinsonian drug and an analgesic drug; (b) at least one second CNS drug which is an NMDA receptor antagonist; and (c) at least one peripheral adrenergic receptor agonist.

The present invention further provides, in another aspect, a unit dosage form, comprising a pharmaceutical composition as described above.

In certain embodiments, the anti-parkinsonian drug is selected from the group consisting of (a) L-3,4-dihydroxyphenylalanine (levodopa); (b) a dopamine agonist selected from the group consisting of bromocriptine, cabergoline, pergolide, pramipexole, ropinirole, piribedil, apomorphine, rigotine, quinagolide, fenoldopam, and lisuride; and (c) a monoamine oxidase B (MAOB) inhibitor selected from the group consisting of selegiline, desmethylselegiline, pargyline, rasagiline, lazabemide, milacemide, mofegiline, D-deprenyl and ladostigil. Each possibility represents a separate embodiment of the present invention. In certain embodiments, the anti-parkinsonian drug is levodopa.

In certain embodiments, the analgesic drug is selected from the group consisting of morphine, amitriptyline, dipyrone, fentanyl, promedolum, omnoponum, oxycodone, hydrocodone, hydromorphone, hydrocodone bitartrate, and buprenorphine. In certain embodiments, the analgesic drug is morphine, amitriptyline or dipyrone. Each possibility represents a separate embodiment of the present invention. In certain embodiments, the analgesic drug is morphine. In certain embodiments, the analgesic drug is amitriptyline. In certain embodiments, the analgesic drug is dipyrone.

In certain embodiments, the NMDA receptor antagonist is selected from the group of memantine, amantadine, dextromethorphan and ketamine Each possibility represents a separate embodiment of the present invention. In certain embodiments, the NMDA receptor antagonist is memantine.

In certain embodiments, the peripheral adrenergic receptor agonist is selected from the group consisting of phenylephrine, epinephrine, midodrine and pseudoephedrine. In certain embodiments, the peripheral adrenergic receptor agonist is phenylephrine or epinephrine. Each possibility represents a separate embodiment of the present invention. In certain embodiments, the peripheral adrenergic receptor agonist is phenylephrine. In certain embodiments, the peripheral adrenergic receptor agonist is epinephrine.

In certain embodiments, the first CNS drug is levodopa, morphine, amitriptyline or dipyrone, the NMDA receptor antagonist is memantine, and the peripheral adrenergic receptor agonist is phenylephrine or epinephrine. Each possibility represents a separate embodiment of the present invention.

In certain embodiments, the first CNS drug is levodopa, the NMDA receptor antagonist is memantine, and the peripheral adrenergic receptor agonist is phenylephrine. In certain embodiments, the first CNS drug is morphine, the NMDA receptor antagonist is memantine, and the peripheral adrenergic receptor agonist is epinephrine. In certain embodiments, the first CNS drug is morphine, the NMDA receptor antagonist is memantine, and the peripheral adrenergic receptor agonist is phenylephrine. In certain embodiments, the first CNS drug is amitriptyline, the NMDA receptor antagonist is memantine, and the peripheral adrenergic receptor agonist is phenylephrine. In certain embodiments, the first CNS drug is dipyrone, the NMDA receptor antagonist is memantine, and the peripheral adrenergic receptor agonist is phenylephrine.

In certain embodiments, the side effect is selected from the group consisting hyperkinesia, sedation, hyperalgesia, catalepsy, dyskinesia, and addiction. Each possibility represents a separate embodiment of the present invention. In certain embodiments, the side effect is hyperkinesia. In certain embodiments, the side effect is sedation. In certain embodiments, the side effect is hyperalgesia. In certain embodiments, the side effect is catalepsy. In certain embodiments, the side effect is dyskinesia. In certain embodiments, the side effect is addiction.

In certain embodiments, the method comprises systemically administering to the subject 5-200 mg levodopa. In certain embodiments, the method comprises systemically administering to the subject 5, 10, 30, 50, 100 or 200 mg levodopa. Each possibility represents a separate embodiment of the present invention.

In certain embodiments, the method comprises systemically administering to the subject 0.1-50 mg morphine. In certain embodiments, the method comprises systemically administering to the subject 0.1, 0.2, 0.5, 1, 2, 4, 5, 10, 25, 30 or 50 mg morphine. Each possibility represents a separate embodiment of the present invention.

In certain embodiments, the method comprises systemically administering to the subject 0.5-20 mg amitriptyline. In certain embodiments, the method comprises systemically administering to the subject 0.5, 1 or 20 mg amitriptyline. Each possibility represents a separate embodiment of the present invention.

In certain embodiments, the method comprises systemically administering to the subject 0.1-40 mg dipyrone. In certain embodiments, the method comprises systemically administering to the subject 0.5, 1 or 40 mg dipyrone. Each possibility represents a separate embodiment of the present invention.

In certain embodiments, the method comprises systemically administering to the subject 5-30 mg memantine. In certain embodiments, the method comprises systemically administering to the subject 5, 10 or 30 mg memantine. Each possibility represents a separate embodiment of the present invention.

In certain embodiments, the method comprises systemically administering to the subject 0.1-3 mg phenylephrine. In certain embodiments, the method comprises systemically administering to the subject 0.1, 0.2, 0.3, 0.5, 1 or 3 mg phenylephrine. Each possibility represents a separate embodiment of the present invention.

In certain embodiments, the method comprises systemically administering to the subject 0.05-0.1 mg epinephrine. In certain embodiments, the method comprises systemically administering to the subject 0.05 or 0.1 mg epinephrine. Each possibility represents a separate embodiment of the present invention.

In certain embodiments, the method comprises systemically administering to the subject (a) 5-200, 5, 10, 30, 50, 100 or 200 mg levodopa, (b) 0.1-50, 0.1, 0.2, 0.5, 1, 2, 4, 5, 10, 25, 30 or 50 mg morphine (c) 0.5-20, 0.5, 1 or 20 mg amitriptyline or (d) 0.5-40, 0.5, 1 or 40 mg dipyrone; (e) 5-30, 5, 10 or 30 mg memantine; and (f) 0.1-3, 0.1, 0.2, 0.3, 0.5, 1 or 3 mg phenylephrine or (g) 0.05-0.1, 0.05 or 0.1 mg epinephrine. Each possibility represents a separate embodiment of the present invention.

In certain embodiments, the method comprises systemically administering to the subject (a) 5-10, 5 or 10 mg levodopa, (b) 0.1-2, 0.1, 0.2, 0.5, 1 or 2 mg morphine, (c) 0.5-1, 0.5 or 1 mg amitriptyline or (d) 0.5-40, 0.5, 1 or 40 mg dipyrone; (e) 5-30, 5, 10 or 30 mg memantine; and/or (f) 0.1-1, 0.1, 0.2, 0.3, 0.5 or 1 mg phenylephrine or (g) 0.05-0.1, 0.05 or 0.1 mg epinephrine. Each possibility represents a separate embodiment of the present invention.

In certain embodiments, the method comprises systemically administering to the subject the first CNS drug, the second CNS drug and the peripheral adrenergic receptor agonist in a molar ratio of 0.2-1000:1-300:1, respectively. Each possibility represents a separate embodiment of the present invention.

In certain embodiments, the pharmaceutical composition described above is for use in a method of potentiating the activity or reducing at least one side effect of first the CNS drug. In certain embodiments, the first CNS drug is an anti-parkinsonian drug, for use in a method of treating Parkinson Disease (PD) or Progressive Supranuclear Palsy (PSP). In certain embodiments, the first CNS drug is an analgesic drug, for use in a method of treating pain or hyperalgesia.

In certain embodiments, the anti-parkinsonian drug is selected from the group consisting L-3,4-dihydroxyphenylalanine (L-DOPA); dopamine agonists selected from the group consisting of bromocriptine, cabergoline, pergolide, pramipexole, ropinirole, piribedil, apomorphine, rigotine, quinagolide, fenoldopam and lisuride; monoamine oxidase B (MAOB) inhibitors selected from the group consisting of selegiline, desmethylselegiline, pargyline, rasagiline, lazabemide, milacemide, mofegiline, D-deprenyl and ladostigil; and an NMDA receptor antagonists selected from the group consisting of memantine, amantadine and ketamine. Each possibility represents a separate embodiment of the present invention. Each possibility represents a separate embodiment of the present invention. In certain embodiments, the anti-parkinsonian drug is L-DOPA.

In certain embodiments, the method described above comprises administering to the subject L-DOPA and a DOPA decarboxylase inhibitor (DDCI) selected from the group consisting of benserazide, (2S)-3-(3,4-dihydroxyphenyl)-2-hydrazino-2-methylpropanoic acid (carbidopa), α-Difluoromethyl-DOPA (DFMD) and L-α-Methyl-3,4-dihydroxyphenylalanine (methyldopa). Each possibility represents a separate embodiment of the present invention. In certain embodiments, the method described above comprises administering to the subject L-DOPA and carbidopa. In certain embodiments, the pharmaceutical composition described above comprises L-DOPA and carbidopa.

In certain embodiments, the analgesic drug is non-opioid analgesic drug selected from the group consisting of: a) a nonsteroidal anti-inflammatory (NSAID) drug selected from the group consisting of dipyrone, piroxicam, paracetamol, naproxen, nabumetone, ketoprofen, diclofenac, ibuprofen, naproxen sodium and aspirin; and b) an antidepressant drug selected from the group consisting of amitriptyline, duloxetine, fluoxetine, milnacipran, and imipramine Each possibility represents a separate embodiment of the present invention.

In certain embodiments, the molar ratio of the first CNS drug, the second CNS drug and the peripheral adrenergic receptor agonist is at least 0.25:at least 1:1, respectively. In certain embodiments, the molar ratio of the first CNS drug, the second CNS drug and the peripheral adrenergic receptor agonist is at least 0.5:at least 2.5:1, respectively. In certain embodiments, the molar ratio of the first CNS drug, the second CNS drug and the peripheral adrenergic receptor agonist is at least 0.2:at least 1:1, respectively.

In certain embodiments, the molar ratio of the first CNS drug, the second CNS drug and the peripheral adrenergic receptor agonist is 0.25-2000:1-600:1, respectively. In certain embodiments, the molar ratio of the first CNS drug, the second CNS drug and the peripheral adrenergic receptor agonist is 0.5-1000:2.5-300:1, respectively. In certain embodiments, the molar ratio of the first CNS drug, the second CNS drug and the peripheral adrenergic receptor agonist is 0.2-1000:1-300:1, respectively. Each possibility represents a separate embodiment of the present invention.

In certain embodiments, the molar ratio of the first CNS drug, the second CNS drug, and the peripheral adrenergic receptor agonist is 4-180:9-30:1, respectively. In certain embodiments, the first CNS drug is levodopa, the second CNS drug is memantine, and the peripheral adrenergic receptor agonist is phenylephrine. In certain embodiments, the method described above comprises administering to the subject 0.025-12 mg phenylephrine. In certain embodiments, the method described above comprises administering to the subject 0.05-6 mg phenylephrine. In certain embodiments, the method described above comprises administering to the subject 0.1-3 mg phenylephrine. In certain embodiments, the method described above comprises administering to the subject 0.3-1 mg phenylephrine. In certain embodiments, the method described above comprises administering to the subject 0.4-400 mg levodopa. In certain embodiments, the method described above comprises administering to the subject 0.75-300 mg levodopa. In certain embodiments, the method described above comprises administering to the subject 1.5-200 mg levodopa. In certain embodiments, the method described above comprises administering to the subject 4-30 mg levodopa. In certain embodiments, the method described above comprises administering to the subject 0.4-40 mg memantine. In certain embodiments, the method described above comprises administering to the subject 0.75-40 mg memantine. In certain embodiments, the method described above comprises administering to the subject 1.5-30 mg memantine. In certain embodiments, the method described above comprises administering to the subject 4-10 mg memantine.

In certain embodiments, the molar ratio of first CNS drug:second CNS drug:peripheral adrenergic receptor agonist is 10-350:50-150:1, respectively. In certain embodiments, the molar ratio of first CNS drug:second CNS drug:peripheral adrenergic receptor agonist is 10-350:103:1, respectively. Each possibility represents a separate embodiment of the present invention. In certain embodiments, the first CNS drug is morphine, the second CNS drug is memantine, and the peripheral adrenergic receptor agonist is epinephrine. In certain embodiments, the method described above comprises administering to the subject 0.004-1.2 mg epinephrine. In certain embodiments, the method described above comprises administering to the subject 0.0075-0.6 mg epinephrine. In certain embodiments, the method described above comprises administering to the subject 0.015-0.3 mg epinephrine. In certain embodiments, the method described above comprises administering to the subject 0.04-0.1 mg epinephrine. In certain embodiments, the method described above comprises administering to the subject 0.04-30 mg morphine. In certain embodiments, the method described above comprises administering to the subject 0.075-30 mg morphine. In certain embodiments, the method described above comprises administering to the subject 0.15-20 mg morphine. In certain embodiments, the method described above comprises administering to the subject 0.4-1 mg morphine. In certain embodiments, the method described above comprises administering to the subject 0.4-40 mg memantine. In certain embodiments, the method described above comprises administering to the subject 0.75-40 mg memantine. In certain embodiments, the method described above comprises administering to the subject 1.5-30 mg memantine. In certain embodiments, the method described above comprises administering to the subject 4-10 mg memantine.

In certain embodiments, the pharmaceutical composition described above comprises the first CNS drug, the second CNS drug and the peripheral adrenergic receptor agonist in a molar ratio of 0.2-1000:1-300:1, respectively. Each possibility represents a separate embodiment of the present invention. In certain embodiments, the pharmaceutical composition described above comprises 0.15-200 mg of the first CNS drug, 1.5-30 mg of the second CNS drug and 0.015-5 mg of the peripheral adrenergic receptor agonist.

In certain embodiments, the pharmaceutical compositions described above are formulated for systemic administration, wherein said systemic administration is independently selected from the group consisting of parenteral, intravenous, intramuscular, subcutaneous, sublingual, rectal and oral administration for each drug. Each possibility represents a separate embodiment of the present invention. In certain embodiments of the methods described above, the first CNS drug, the second CNS drug and the peripheral adrenergic receptor agonist are each administered by the same technique of administration or by different techniques of administration.

In certain embodiments, the molar ratio of the first CNS drug, the second CNS drug and the peripheral adrenergic receptor agonist is 0.25-4000:0.25-800:1, respectively. In certain embodiments, the molar ratio of the first CNS drug, the second CNS drug and the peripheral adrenergic receptor agonist is 0.5-2000:0.5-400:1, respectively. In certain embodiments, the molar ratio of the first CNS drug, the second CNS drug and the peripheral adrenergic receptor agonist is 0.2-1000:1-300:1, respectively.

FDA guidelines (Guidance for Industry, “Estimating the Maximum Safe Starting Dose in Initial Clinical Trials for Therapeutics in Adult Healthy Volunteers”, July 2005, page 7) are routinely used to convert animal doses in mg/kg to human equivalent doses (HED) in mg/kg based on body surface area, assuming a human adult weighing 60 kg and a child weighing 20 kg. In certain embodiments, dosages refer to a human adult subject weighing 60 kg. In certain embodiments, dosages correspond to the dosages of a human adult subject weighing 60 kg. The phrase “correspond to the dosages of a human adult subject weighing 60 kg” as used herein refer to a dosage which is calculated, or derived, from a dosage calculated for a 60 kg human adult subject. Such calculations/derivations are known in the art (see FDA guidelines above) and are routinely practiced by persons with average skill in the art. In certain embodiments, “dosages” refer to the dosage of a single administration.

In certain embodiments, the molar ratio of first CNS drug:second CNS drug:peripheral adrenergic receptor agonist is 4-180:9-30:1, respectively. In certain embodiments, the first CNS drug is levodopa, the second CNS drug is memantine, and the peripheral adrenergic receptor agonist is phenylephrine. In certain embodiments, the method described above comprises administering to the subject 0.025-12 mg phenylephrine. In certain embodiments, the method described above comprises administering to the subject 0.05-6 mg phenylephrine. In certain embodiments, the method described above comprises administering to the subject 0.1-3 mg phenylephrine. In certain embodiments, the method described above comprises administering to the subject 0.3-1 mg phenylephrine. In certain embodiments, the method described above comprises administering to the subject 0.3-4 mg phenylephrine. In certain embodiments, the method described above comprises administering to the subject 0.3, 0.5 or 1 mg phenylephrine. In certain embodiments, the method described above comprises administering to the subject 0.5-400 mg levodopa. In certain embodiments, the method described above comprises administering to the subject 0.75-300 mg levodopa. In certain embodiments, the method described above comprises administering to the subject 1.5-200 mg levodopa. In certain embodiments, the method described above comprises administering to the subject 4-30 mg levodopa. In certain embodiments, the method described above comprises administering to the subject 0.4-40 mg memantine. In certain embodiments, the method described above comprises administering to the subject 0.75-40 mg memantine. In certain embodiments, the method described above comprises administering to the subject 1.5-30 mg memantine. In certain embodiments, the method described above comprises administering to the subject 4, 5, 10 or 30 mg memantine. In certain embodiments, the method described above comprises administering to the subject 4-10 mg memantine. In certain embodiments, the method described above comprises administering to the subject 4, 5 or 10 mg memantine.

In certain embodiments, the molar ratio of first CNS drug:second CNS drug:peripheral adrenergic receptor agonist is 10-350:50-150:1, respectively. In certain embodiments, the first CNS drug is morphine, the second CNS drug is memantine, and the peripheral adrenergic receptor agonist is epinephrine. In certain embodiments, the method described above comprises administering to the subject 0.004-1.2 mg epinephrine. In certain embodiments, the method described above comprises administering to the subject 0.0075-0.6 mg epinephrine. In certain embodiments, the method described above comprises administering to the subject 0.015-0.3 mg epinephrine. In certain embodiments, the method described above comprises administering to the subject 0.04-0.1 mg epinephrine. In certain embodiments, the method described above comprises administering to the subject 0.04, 0.05 or 0.1 mg epinephrine. In certain embodiments, the method described above comprises administering to the subject 0.04-50 mg morphine. In certain embodiments, the method described above comprises administering to the subject 0.04-30 mg morphine. In certain embodiments, the method described above comprises administering to the subject 0.15-50 mg morphine. In certain embodiments, the method described above comprises administering to the subject 0.15-30 mg morphine. In certain embodiments, the method described above comprises administering to the subject 0.4, 0.5, 1, 10, 25 or 50 mg morphine. In certain embodiments, the method described above comprises administering to the subject 0.4, 0.5, 1, 10, 25 or 30 mg morphine.

In certain embodiments, the method described above comprises administering to the subject 0.4, 0.5 or 1 mg morphine. In certain embodiments, the method described above comprises administering to the subject 0.075-30 mg morphine. In certain embodiments, the method described above comprises administering to the subject 0.15-20 mg morphine. In certain embodiments, the method described above comprises administering to the subject 0.4-1 mg morphine. In certain embodiments, the method described above comprises administering to the subject 0.4-40 mg memantine. In certain embodiments, the method described above comprises administering to the subject 0.75-40 mg memantine. In certain embodiments, the method described above comprises administering to the subject 1.5-30 mg memantine. In certain embodiments, the method described above comprises administering to the subject 4-10 mg memantine. In certain embodiments, the method described above comprises administering to the subject 4, 5 or 10 mg memantine.

In certain embodiments, the pharmaceutical composition described above comprises the first CNS drug, the second CNS drug and the peripheral adrenergic receptor agonist in a molar ratio of 0.2-1000:1-300:1, respectively. In certain embodiments, the pharmaceutical composition described above comprises 0.15-200 mg of the first CNS drug, 1.5-30 mg of the second CNS drug and 0.015-5 mg of the peripheral adrenergic receptor agonist.

Preferred dose ranges of active ingredients in compositions for systemic intramuscular administration are as follows: for morphine: from 0.05 mg/kg to 5 mg/kg; for epinephrine: from 0.005 mg/kg to 0.01 mg/kg and for memantine from 0.5 mg/kg to 1 mg/kg. In humans, calculated doses for morphine are from 0.5 mg to 50 mg; for epinephrine from 0.05 mg to 0.1 mg; and for memantine from 0.5 mg to 1.0 mg. In certain embodiments, the method comprises intramuscular administration of 0.5 mg to 50 mg morphine, 0.05 mg to 0.1 mg epinephrine, and 0.5 mg to 1.0 mg memantine.

Preferred dose ranges of active ingredients in compositions for systemic intramuscular administration are as follows: for morphine: from 0.01 mg/kg to 5 mg/kg; for epinephrine: 0.01 mg/kg and for memantine 1 mg/kg. In humans, calculated doses for morphine are from 0.1 mg to 50 mg; for epinephrine 0.1 mg; and for memantine 10 mg. In certain embodiments, the method comprises intramuscular administration of 0.1 mg to 50 mg morphine, 0.1 mg epinephrine, and 10 mg memantine.

Preferred dose ranges of active ingredients in compositions for systemic intramuscular administration are as follows: for morphine: from 0.01 mg/kg to 5 mg/kg; for phenylephrine: 0.02 mg/kg and for memantine 1 mg/kg. In humans, calculated doses for morphine are from 0.1 mg to 50 mg; for phenylephrine 0.2 mg; and for memantine 10 mg. In certain embodiments, the method comprises intramuscular administration of 0.1 mg to 50 mg morphine, 0.2 mg phenylephrine, and 10 mg memantine.

Preferred dose ranges of active ingredients in compositions for systemic intramuscular administration are as follows: for amitriptyline: from 0.05 mg/kg to 10 mg/kg; for phenylephrine: 0.02 mg/kg and for memantine 1 mg/kg. In humans, calculated doses for amitriptyline are from 0.5 mg to 100 mg; for phenylephrine 0.2 mg; and for memantine 10 mg. In certain embodiments, the method comprises intramuscular administration of 0.5 mg to 100 mg amitriptyline, 0.2 mg phenylephrine, and 10 mg memantine.

Preferred dose ranges of active ingredients in compositions for systemic intramuscular administration are as follows: for dipyrone: from 0.05 mg/kg to 20 mg/kg; for phenylephrine: 0.02 mg/kg and for memantine 1 mg/kg. In humans, calculated doses for dipyrone are from 0.5 mg to 200 mg; for phenylephrine 0.2 mg; and for memantine 10 mg. In certain embodiments, the method comprises intramuscular administration of 0.5 mg to 200 mg dipyrone, 0.2 mg phenylephrine, and 10 mg memantine.

Preferred concentration ranges of active ingredients in compositions for systemic oral administration are as follows: for levodopa, from 0.5 mg/kg to 20 mg/kg; for phenylephrine, 0.1 mg/kg; and for memantine, 1 mg/kg. In humans, calculated doses are for levodopa, from 5 mg to 200 mg; for phenylephrine, 1 mg; and for memantine 10 mg. In certain embodiments, the method comprises oral administration of 5 mg to 200 mg levodopa, 1 mg phenylephrine, and 10 mg memantine.

Preferred concentration ranges of active ingredients in compositions for systemic oral administration are as follows: for levodopa, from 5 mg/kg to 20 mg/kg; for phenylephrine, 0.3 mg/kg; and for memantine, 1 mg/kg. In humans, calculated doses are for levodopa, from 50 mg to 200 mg for phenylephrine, 3 mg; and for memantine 10 mg. In certain embodiments, the method comprises oral administration of 50 mg to 200 mg levodopa, 3 mg phenylephrine, and 10 mg memantine.

The present invention provides, in one aspect, a method of potentiating the activity and/or reducing at least one side effect of a CNS drug selected from the group consisting of an anti-parkinsonian drug and an analgesic drug in a subject in need, comprising systemically administering to the subject at least one CNS drug, selected from the group consisting of an anti-parkinsonian drug and an analgesic drug; and at least one peripheral adrenergic receptor agonist; wherein the molar ratio of the CNS drug and peripheral adrenergic receptor agonist is in the range of 1-2000:1, respectively.

The present invention further provides, in another aspect, a method of potentiating the activity or reducing at least one side effect of a CNS drug selected from the group consisting of an anti-parkinsonian drug and an analgesic drug in a subject in need, comprising systemically administering to the subject (a) at least one CNS drug, wherein the CNS drug is levodopa; and (b) at least one peripheral adrenergic receptor agonist, wherein the peripheral adrenergic receptor agonist is phenylephrine or epinephrine; wherein the molar ratio of the CNS drug and peripheral adrenergic receptor agonist is in the range of 1-2000:1, respectively.

The present invention provides, in another aspect, a method of treating Parkinson Disease (PD) or Progressive Supranuclear Palsy (PSP) in a subject in need, comprising systemically administering to the subject at least one CNS drug, which is an anti-parkinsonian drug; and at least one peripheral adrenergic receptor agonist; wherein the molar ratio of the CNS drug and peripheral adrenergic receptor agonist is in the range 1-2000:1, respectively.

The present invention further provides, in another aspect, a method of treating Parkinson Disease (PD) or Progressive Supranuclear Palsy (PSP) in a subject in need, comprising systemically administering to the subject (a) at least one CNS drug, wherein the CNS drug is levodopa; and (b) at least one peripheral adrenergic receptor agonist, wherein the peripheral adrenergic receptor agonist is phenylephrine or epinephrine; wherein the molar ratio of the CNS drug and peripheral adrenergic receptor agonist is in the range of 1-2000:1, respectively.

The present invention provides, in another aspect, a method of treating pain or hyperalgesia in a subject in need, comprising systemically administering to the subject at least one CNS drug, which is an analgesic drug; and at least one peripheral adrenergic receptor agonist; wherein the molar ratio of the CNS drug and peripheral adrenergic receptor agonist is in the range of 1-2000:1, respectively.

The present invention provides, in another aspect, a pharmaceutical composition, comprising at least one CNS drug, selected from the group consisting of an anti-parkinsonian drug and an analgesic drug; and at least one peripheral adrenergic receptor agonist; wherein the molar ratio of the CNS drug and the peripheral adrenergic receptor agonist is in the range of 1-2000:1, respectively.

The present invention further provides, in another aspect, a pharmaceutical composition, comprising (a) at least one CNS drug, wherein the CNS drug is levodopa; and (b) at least one peripheral adrenergic receptor agonist, wherein the peripheral adrenergic receptor agonist is phenylephrine or epinephrine; wherein the molar ratio of the CNS drug and the peripheral adrenergic receptor agonist is in the range of 1-2000:1, respectively.

The present invention further provides, in another aspect, a unit dosage form, comprising a pharmaceutical composition as described above.

In certain embodiments, the CNS drug is levodopa and the peripheral adrenergic receptor agonist is phenylephrine or epinephrine. Each possibility represents a separate embodiment of the present invention. In certain embodiments, the CNS drug is levodopa and the peripheral adrenergic receptor agonist is phenylephrine. In certain embodiments, the CNS drug is levodopa and the peripheral adrenergic receptor agonist is epinephrine.

In certain embodiments, the side effect is selected from the group consisting hyperkinesia, sedation, hyperalgesia, catalepsy, dyskinesia, and addiction. In certain embodiments, the side effect is hyperkinesia. In certain embodiments, the side effect is sedation. In certain embodiments, the side effect is hyperalgesia. In certain embodiments, the side effect is catalepsy. In certain embodiments, the side effect is dyskinesia. In certain embodiments, the side effect is addiction.

In certain embodiments, the method comprises systemically administering to the subject 15-200 mg levodopa. In certain embodiments, the method comprises systemically administering to the subject 15, 30, 100 or 200 mg levodopa.

In certain embodiments, the peripheral adrenergic receptor agonist is phenylephrine. In certain embodiments, the method comprises systemically administering to the subject 0.2-3 mg phenylephrine. In certain embodiments, the method comprises systemically administering to the subject 0.2, 1 or 3 mg phenylephrine.

In certain embodiments, the peripheral adrenergic receptor agonist is epinephrine. In certain embodiments, the method comprises systemically administering to the subject 0.1-0.3 mg epinephrine. In certain embodiments, the method comprises systemically administering to the subject 0.1, 0.2 or 0.3 mg epinephrine.

In certain embodiments, the method comprises systemically administering to the subject (a) 15-30, 15 or 30 mg levodopa; and (b) 0.2-3, 0.2, 1 or 3 mg phenylephrine or (c) 0.1 mg epinephrine.

In certain embodiments, the method comprises systemically administering to the subject (a) 15 mg levodopa; and/or (b) 0.2-1, 0.2 or 1 mg phenylephrine and/or (c) 0.1 mg epinephrine.

In certain embodiments, the pharmaceutical composition described above is for use in a method of potentiating the activity or reducing at least one side effect of the CNS drug. In certain embodiments, the pharmaceutical composition is for use in a method of treating Parkinson Disease (PD) or Progressive Supranuclear Palsy (PSP).

The phrase “potentiating the activity” as used herein generally means increasing the biological activity and/or increasing the therapeutic efficacy of a CNS drug. The phrase “reducing at least one side effect” as used herein generally means decreasing the likelihood of appearance, postponing the appearance and/or decreasing the severity of at least one side effect associated with or caused by a CNS drug. The term “CNS drug” as used herein generally relates to drugs and classes of drugs acting on the human central nervous system.

The term “anti-parkinsonian drug” as used herein relates to any drug which prevents, alleviates or treats at least one symptom of Parkinson's disease (PD) or Progressive Supranuclear Palsy (PSP) or Parkinsonism syndrome. The term “analgesic drug” as used herein relates to any drug which prevents or alleviates pain. The term “peripheral adrenergic receptor agonist” as used herein relates to any drug acting on one or more peripheral adrenoreceptors. The term “subject” as used herein relates to an animal, preferably a mammal, most preferably a human, who is in the need of prevention or treatment of PD, PSP, pain or hyperalgesia. The term “treating” as used herein means to prevent or ameliorate one or more symptoms associated with the referenced symptom, disorder, disease or condition. The term “pharmaceutical composition” as used herein refers to any composition comprising at least one pharmaceutically active ingredient and at least one other ingredient, as well as to any product which results, directly or indirectly, from combination, complexation, or aggregation of the two or more the ingredients, or from dissociation of one or more of the ingredients. Accordingly, the term “pharmaceutical composition” as used herein may encompass, inter alia, any composition made by admixing a pharmaceutically active ingredient and one or more pharmaceutically acceptable carriers. Non-limiting examples of pharmaceutically active ingredients are CNS drugs and peripheral adrenergic receptor agonists. The term “pharmaceutically acceptable carrier” refers to a carrier for administration of a therapeutic agent. Such carriers include, but are not limited to, saline, buffered saline, dextrose, water, glycerol, ethanol, and combinations thereof.

The term “molar ratio” as used herein relates to the respective molar ratio between at least one CNS drug and at least one peripheral adrenergic receptor agonist which are found in the same pharmaceutical composition, or which are separately administered to a subject.

As used herein, the term “systemic administration” refers to a route of administration that is, e.g., enteral or parenteral, and results in the systemic distribution of an active agent leading to systemic absorption or accumulation of active agents in the blood stream followed by distribution throughout the entire body. Suitable unit dosage forms, in part, depend upon the use or the route of entry, for example oral, transdermal, or by injection. Administration routes which lead to systemic circulation include, but not limited to, oral, intramuscular, intravenous, subcutaneous, intraperitoneal, inhalation and intrapulmonary and administration.

In certain embodiments, the systemic administration is oral or intramuscular administration. In certain embodiments, the systemic administration is oral administration. In certain embodiments, the systemic administration is intramuscular administration.

Preferred concentration ranges of active ingredients in compositions for systemic oral administration are as follows: for levodopa, from 1.5 mg/kg to 20 mg/kg; and for phenylephrine, from 0.1 mg/kg to 0.3 mg/kg. In humans, the calculated doses range for levodopa from 15 mg to 200 mg, and for phenylephrine from 1 mg to 3 mg. In certain embodiments, the method comprises oral administration of 15 mg to 200 mg levodopa and 1 mg to 3 mg phenylephrine.

Preferred dose ranges of active ingredients in compositions for intramuscular administration are as follows: for morphine: from 0.2 mg/kg to 5 mg/kg; and for epinephrine: from 0.01 mg/kg to 0.03 mg/kg. In humans, calculated doses for the morphine: from 2 mg to 50 mg; and for epinephrine: from 0.05 mg to 0.3 mg. In certain embodiments, the method comprises intramuscular administration of 2 mg to 50 mg morphine and 0.05 mg to 0.3 mg epinephrine.

Preferred dose ranges of active ingredients in compositions for oral administration are as follows: for morphine: from 0.1 mg/kg to 3 mg/kg; and for phenylephrine: from 0.02 mg/kg to 0.1 mg/kg. In humans, calculated doses for morphine: from 1 mg to 30 mg; and for phenylephrine: from 0.2 mg to 1 mg. In certain embodiments, the method comprises oral administration of 1 mg to 30 mg morphine and 0.2 mg to 1 mg phenylephrine.

Preferred dose ranges of active ingredients in compositions for intramuscular administration are as follows: for morphine: from 0.2 mg/kg to 5 mg/kg; and for epinephrine: 0.01 mg/kg. In humans, calculated doses for morphine: from 2 mg to 50 mg; and for epinephrine: 1 mg. In certain embodiments, the method comprises intramuscular administration of 2 mg to 50 mg morphine and 1 mg epinephrine.

Preferred dose ranges of active ingredients in compositions for intramuscular administration are as follows: for memantine from 1 mg/kg to 15 mg/kg; for epinephrine: 0.01 mg/kg. In humans, calculated doses for memantine: from 10 mg to 150 mg; and for epinephrine: 1 mg. In certain embodiments, the method comprises intramuscular administration of 10 mg to 150 mg memantine and 1 mg epinephrine.

Preferred dose ranges of active ingredients in compositions for oral administration are as follows: for morphine: from 0.1 mg/kg to 5 mg/kg; and for phenylephrine: 0.02 mg/kg. In humans, calculated doses for morphine: from 1 mg to 50 mg; and for phenylephrine: 0.2 mg. In certain embodiments, the method comprises oral administration of 1 mg to 50 mg morphine and 0.2 mg phenylephrine.

Preferred dose ranges of active ingredients in compositions for oral administration are as follows: for memantine: from 1 mg/kg to 20 mg/kg; and for phenylephrine: 0.02 mg/kg. In humans, calculated doses for memantine: from 10 mg to 200 mg; and for phenylephrine: 0.2 mg. In certain embodiments, the method comprises oral administration of 10 mg to 200 mg memantine and 0.2 mg phenylephrine.

Preferred dose ranges of active ingredients in compositions for oral administration are as follows: for amitriptyline: from 0.1 mg/kg to 10 mg/kg; for phenylephrine: 0.02 mg/kg. In humans, calculated doses for amitriptyline: from 1 mg to 100 mg; and for phenylephrine: 0.2 mg. In certain embodiments, the method comprises oral administration of 1 mg to 100 mg amitriptyline and 0.2 mg phenylephrine.

Preferred dose ranges of active ingredients in compositions for oral administration are as follows: for dipyrone: from 0.2 mg/kg to 20 mg/kg; and for phenylephrine: 0.02 mg/kg. In humans, calculated doses for dipyrone: from 2 mg to 200 mg; and for phenylephrine: 0.2 mg. In certain embodiments, the method comprises oral administration of 2 mg to 200 mg dipyrone and 0.2 mg phenylephrine.

Preferred concentration ranges of active ingredients in compositions for systemic oral administration are as follows: for levodopa, from 1.5 mg/kg to 20 mg/kg; and for phenylephrine, from 0.1 mg/kg to 0.3 mg/kg. In humans, the calculated doses range for levodopa: from 15 mg to 200 mg, and for phenylephrine, from 1 mg to 3 mg. In certain embodiments, the method comprises oral administration of 15 mg to 200 mg levodopa and 1 mg to 3 mg phenylephrine.

Preferred concentration ranges of active ingredients in compositions for systemic oral administration are as follows: for memantine, from 0.5 mg/kg to 10 mg/kg; for phenylephrine 0.1 mg/kg. In humans, the calculated doses range for memantine from 5 mg to 100 mg, and for phenylephrine 1 mg. In certain embodiments, the method comprises oral administration of 5 mg to 100 mg memantine and 1 mg phenylephrine.

In certain embodiments, the molar ratio of the CNS drug and peripheral adrenergic receptor agonist is in the range of 2-1000:1, respectively. In certain embodiments, the molar ratio of the CNS drug and peripheral adrenergic receptor agonist is in the range of 5-500:1, respectively.

In certain embodiments, the molar ratio of the CNS drug:peripheral adrenergic receptor agonist is 1-1000:1, respectively. In certain embodiments, the molar ratio of the CNS drug:peripheral adrenergic receptor agonist is 2.5-400:1, respectively. In certain embodiments, the molar ratio of the CNS drug:peripheral adrenergic receptor agonist is 5-200:1, respectively. In certain embodiments, the CNS drug is levodopa and the peripheral adrenergic receptor agonist is phenylephrine. In certain embodiments, the method described above comprises administering to the subject 0.05-20 mg phenylephrine. In certain embodiments, the method described above comprises administering to the subject 0.15-6 mg phenylephrine. In certain embodiments, the method described above comprises administering to the subject 0.3-3 mg phenylephrine. In certain embodiments, the method described above comprises administering to the subject 0.2-12 mg phenylephrine. In certain embodiments, the method described above comprises administering to the subject 0.45-6 mg phenylephrine. In certain embodiments, the method described above comprises administering to the subject 0.9-3 mg phenylephrine. In certain embodiments, the method described above comprises administering to the subject 1 or 3 mg phenylephrine. In certain embodiments, the method described above comprises administering to the subject 1-5 mg phenylephrine. In certain embodiments, the method described above comprises administering to the subject 1-400 mg levodopa. In certain embodiments, the method described above comprises administering to the subject 2.5-400 mg levodopa. In certain embodiments, the method described above comprises administering to the subject 5-200 mg levodopa. In certain embodiments, the method described above comprises administering to the subject 5-30 mg levodopa. In certain embodiments, the method described above comprises administering to the subject 30 mg levodopa. In certain embodiments, the method described above comprises administering to the subject 25-200 mg levodopa. In certain embodiments, the method described above comprises administering to the subject 5-30 mg levodopa.

In certain embodiments, the molar ratio of the CNS drug:peripheral adrenergic receptor agonist is 1-1300:1, respectively. In certain embodiments, the molar ratio of the CNS drug:peripheral adrenergic receptor agonist is 3-640:1, respectively. In certain embodiments, the molar ratio of the CNS drug:peripheral adrenergic receptor agonist is 6-322:1, respectively. In certain embodiments, the CNS drug is morphine and the peripheral adrenergic receptor agonist is epinephrine. In certain embodiments, the method described above comprises administering to the subject 0.005-2 mg epinephrine. In certain embodiments, the method described above comprises administering to the subject 0.015-0.6 mg epinephrine. In certain embodiments, the method described above comprises administering to the subject 0.05-0.3 mg epinephrine. In certain embodiments, the method described above comprises administering to the subject 0.05 mg epinephrine. In certain embodiments, the method described above comprises administering to the subject 0.05, 0.1 or 0.3 mg epinephrine. In certain embodiments, the method described above comprises administering to the subject 0.05 or 0.1 mg epinephrine. In certain embodiments, the method described above comprises administering to the subject 0.05-0.1 mg epinephrine. In certain embodiments, the method described above comprises administering to the subject 0.3 mg epinephrine. In certain embodiments, the method described above comprises administering to the subject 0.5-30 mg morphine. In certain embodiments, the method described above comprises administering to the subject 0.15-15 mg morphine. In certain embodiments, the method described above comprises administering to the subject 0.3-8 mg morphine. In certain embodiments, the method described above comprises administering to the subject 1-4 mg morphine. In certain embodiments, the method described above comprises administering to the subject 0.5-4 mg morphine. In certain embodiments, the method described above comprises administering to the subject 0.5-1 mg morphine.

In certain embodiments, the molar ratio of the CNS drug:peripheral adrenergic receptor agonist is 1-350:1, respectively. In certain embodiments, the molar ratio of the CNS drug:peripheral adrenergic receptor agonist is 2.5-180:1, respectively. In certain embodiments, the molar ratio of the CNS drug:peripheral adrenergic receptor agonist is 5-100:1, respectively. In certain embodiments, CNS drug is morphine and the peripheral adrenergic receptor agonist is phenylephrine. In certain embodiments, the method described above comprises administering to the subject 0.15-20 mg phenylephrine. In certain embodiments, the method described above comprises administering to the subject 0.2-4 mg phenylephrine. In certain embodiments, the method described above comprises administering to the subject 0.2-1 mg phenylephrine. In certain embodiments, the method described above comprises administering to the subject 0.2 or 1 mg phenylephrine. In certain embodiments, the method described above comprises administering to the subject 1-10 mg phenylephrine. In certain embodiments, the method described above comprises administering to the subject 0.05-4 mg phenylephrine. In certain embodiments, the method described above comprises administering to the subject 0.5-30 mg morphine. In certain embodiments, the method described above comprises administering to the subject 0.15-8 mg morphine. In certain embodiments, the method described above comprises administering to the subject 0.3-4 mg morphine. In certain embodiments, the method described above comprises administering to the subject 0.5-2 mg morphine.

In certain embodiments, the pharmaceutical composition described above comprises 0.15-200 mg of the CNS drug and 0.005-20 mg of the peripheral adrenergic receptor agonist. In certain embodiments, the pharmaceutical composition described above comprises 0.3-100 mg of the CNS drug and 0.015-10 mg of the peripheral adrenergic receptor agonist. In certain embodiments, the pharmaceutical composition described above comprises 0.5-50 mg of the CNS drug and 0.02-5 mg of the peripheral adrenergic receptor agonist. In certain embodiments, the pharmaceutical composition described above comprises 0.6-30 mg of the CNS drug and 0.3-5 mg of the peripheral adrenergic receptor agonist.

The term “and/or” as used herein means “either or both of”. The phrase “at least one of ‘A’ and ‘B’,” as used herein means “any group having at least one ‘A’; or any group having at least one ‘B’.

In certain embodiments, at least one drug is administered in a low dose, which is 10 fold to 100 fold lower than its standard dose. In certain embodiments, at least one drug is administered in a sub-standard dose, which is 2 fold to 5 fold lower than its standard dose. In certain embodiments, at least one drug is administered in its standard dose.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

The following examples are presented in order to more fully illustrate some embodiments of the invention. They should, in no way be construed, however, as limiting the broad scope of the invention.

### EXAMPLES

### Example 1. Potentiation of the Anti-Parkinsonian Effect and Reduction of the Side Effects of Levodopa in a Haloperidol Catalepsy Model (Oral Administration)

### Methods.

Anti-parkinsonian effect of drugs was investigated on a model of induced catalepsy by I.M. injection of haloperidol at a dose of 1 mg/kg in Wistar rats (weight 160-170 g). Catalepsy was evaluated by duration of immobilization of rats positioned on a large grid fixed at an angle of 45 degrees 60 minutes after the haloperidol administration. Maximum catalepsy in rats was recorded in the case of their complete immobilization on grid after 180 seconds of observation (Campbell A., et al., 1988, Neuropharmacology, Vol. 27(11), pages 1197-1199; Ossowska K. J., Neural. Transm. Park. Dis. Dement. Sect., 1994, Vol. 8(1-2), pages 39-71; Gmiro V. E. and Serdyuk S. E., Bulletin of Experimental Biology and Medicine, 2007, Vol. 143 (5), pages 554-556).

Levodopa, memantine, phenylephrine, as well as ternary combinations of levodopa with phenylephrine and memantine were administered orally in a volume of 1.0 ml through a rigid metal probe, 45 minutes before the administration of haloperidol. Control animals received orally 1 ml of distilled water.

Anti-parkinsonian drugs effects were estimated as decrease of the average duration of immobilization of animals treated by test compositions compared to a control (DW).

### Open Field Test.

To evaluate the (hyperkinetic) side effects of tested drugs, an “open field” (OF) test was applied. In the OF test, the locomotive activity of rats is determined Animals were placed in the center of square of the illuminated field (1 meter×1 meter) for 3 minutes and the mobility time was recorded in seconds (horizontal activity). The registration of horizontal locomotive activity was performed 40 minutes after administration of the test drug(s), 5 minutes before the administration of haloperidol. To quantify the locomotive activity for each dose of test drug(s) an average horizontal activity was calculated. Hyperkinetic effect of levodopa, phenylephrine, memantine, as well as of combinations with levodopa, phenylephrine and memantine was evaluated by an average increase in horizontal activity in the OF test in % compared with measures for rats in the control group, and also as the number of rats with significant increase in horizontal activity (50% and more as compared to control).

Intramuscular (IM) injection of haloperidol at a dose of 1 mg/kg, 60 minutes after injection, results in the control group in immobilization of 158.2±25 seconds (Table 1).

TABLE 1

| Levodopa | Memantine | Phenylephrine | Time on grid | Hyperkinesia | Levodopa | Memantine | Phenylephrine |
|---|---|---|---|---|---|---|---|
| (Rat mg/kg) | (Rat mg/kg) | (Rat mg/kg) | (seconds) | (% of Rats) | (mg, Human Dose) ** | (mg, Human Dose) ** | (mg, Human Dose) ** |
| | | | 158.2* | 0* | | | |
| 1 | | | 147 | 0 | 9.6 | | |
| 3 | | | 125 | 0 | 29 | | |
| 10 | | | 85 | 50 | 96.7 | | |
| 20 | | | 35 | 100 | 193.5 | | |
| | | 0.1 | 137 | 0 | | | 0.9 |
| | | 0.3 | 98 | 0 | | | 2.9 |
| | | 1 | 78 | 0 | | | 9.6 |
| | 1 | | 139 | 0 | | 9.6 | |
| | 1 | 0.1 | 79 | 0 | | 9.6 | 0.9 |
| | 3 | | 108 | 0 | | 29 | |
| | 10 | | 65 | 50 | | 96.7 | |
| | 20 | | 34 | 100 | | 193.5 | |
| 0.5 | 0.5 | 0.03 | 38 | 0 | 4.83 | 4.83 | 0.29 |
| 0.5 | 1 | 0.1 | 28 | 0 | 4.83 | 9.67 | 0.96 |
| 1 | 1 | 0.05 | 33 | 0 | 9.67 | 9.67 | 0.48 |
| 3 | 1 | 0.1 | 12 | 0 | 29.03 | 9.67 | 0.96 |
| 5 | 3 | 0.1 | 12 | 0 | 48.38 | 29.03 | 0.96 |
| 10 | 1 | 0.1 | 6 | 0 | 96.77 | 9.67 | 0.96 |
| 10 | 3 | 0.1 | 5 | 0 | 96.77 | 29.03 | 0.96 |
| 20 | 1 | 0.1 | 3 | 0 | 193.54 | 29.67 | 0.96 |

*N.C.—Negative control (distilled water).
** Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).

### Triple-Therapy.

Combined levodopa administration in a dose of 0.5 mg/kg with memantine in the low dose of 0.5 mg/kg and phenylephrine at a low dose of 0.03 mg/kg caused a significant, nearly maximal anti-parkinsonian effect (reduced the duration of immobilization on the grid by a factor of 4, from 158 to 38 seconds), comparable to levodopa and memantine in the high dose of 20 mg/kg for independent use. This combination has not increased the horizontal activity of rats. Consequently, compositions comprising memantine in a low threshold dose of 0.5 mg/kg and phenylephrine in a low threshold dose 0.03 mg/kg reduced the effective dose of levodopa causing maximal anti-parkinsonian effect without developing hyperkinetic side effect by 40 times (20.0 mg/kg versus 0.5 mg/kg).

Combined levodopa administration in a dose of 0.5 mg/kg with memantine in the threshold dose of 1 mg/kg and phenylephrine at a threshold dose of 0.1 mg/kg reached a maximal anti-parkinsonian effect (reduced the duration of immobilization on the grid by a factor of 5.6, from 158 to 28 seconds), that is 1.2 fold stronger than levodopa alone in the high dose of 20 mg/kg. This combination has not increased the horizontal activity of rats.

Combined levodopa administration in a dose of 1 mg/kg with memantine in the dose of 1 mg/kg and phenylephrine at a low dose of 0.05 mg/kg caused a significant anti-parkinsonian effect (reduced the duration of immobilization on the grid by a factor of 4.7, from 158 to 33 seconds), comparable to levodopa alone or memantine alone in the high dose of 20 mg/kg. This combination has not increased the horizontal activity of rats.

Combined administration of levodopa in conventional low dose of 3 mg/kg with memantine in the threshold dose of 1 mg/kg and phenylephrine at a threshold dose of 0.1 mg/kg leads to a 2.8 fold increases in the maximal anti-parkinsonian effect compared to levodopa at a dose of 20 mg/kg (reduced immobilization duration the grid from 35 to 12 seconds), without development hyperkinesia in rats. In comparison, the composition of memantine in the threshold dose 1 mg/kg with phenylephrine at a threshold dose of 0.1 mg/kg caused only a mild anti-parkinsonian effect, as it reduced the amount of haloperidol catalepsy from 158 to 79 seconds, while in combining with oral administration of levodopa on the one hand multiplies the maximal anti-parkinsonian effect of levodopa, and on the other hand reduced by 7 times (to 3 mg/kg) the effective dose of levodopa causing potentiated anti-parkinsonian effect without developing side hyperkinetic effects. It is highly surprising that the potentiated synergy upon combined administration of levodopa within a triple composition with phenylephrine 0.1 mg/kg and memantine 1.0 mg/kg, results in a multiple increase in anti-parkinsonian action and 7 fold reduction of the effective dose of levodopa causing potentiated effect.

Combined levodopa administration in a dose of 5 mg/kg with memantine in the dose of 3 mg/kg and phenylephrine at a threshold dose of 0.1 mg/kg caused a maximal anti-parkinsonian effect (reduced the duration of immobilization on the grid by a factor of 13, from 158 to 12 seconds), that is 3 fold stronger than levodopa alone in the high dose of 20 mg/kg. Such a combination has not increased the horizontal activity of rats.

Combined levodopa administration in a dose of 10 mg/kg with memantine in the dose of 3 mg/kg and phenylephrine at a threshold dose of 0.1 mg/kg caused a maximal anti-parkinsonian effect (reduced the duration of immobilization on the grid by a factor of 31, from 158 to 5 seconds), that is 7 fold stronger than levodopa alone in the high dose of 20 mg/kg. Such a combination has not increased the horizontal activity of rats.

Combined administration of levodopa in medium therapeutic dose of 10 mg/kg and in the highest dose of 20 mg/kg with memantine in threshold dose of 1 mg/kg and phenylephrine at a threshold dose of 0.1 mg/kg increased the maximum anti-parkinsonian effect by a factor of 5-8 compared to levodopa alone in a dose of 20 mg/kg (immobilization duration on the grid decreases from 35 to 6 and 3 seconds, respectively) and also eliminated hyperkinesia in all rats.

The results of these studies indicate that ternary compositions containing levodopa, phenylephrine, and memantine exert an anti-parkinsonian effect which is vastly superior to the anti-parkinsonian effect of levodopa in the conventional dose, without developing hyperkinetic side effect, and significantly reduce the dosage of conventional levodopa, memantine and phenylephrine in the composition. The above-mentioned compositions cause a potentiation of the anti-parkinsonian action of levodopa in a safe manner, since it eliminates the side effects of the use of each of the components of the composition.

### Example 2. Potentiation of Analgesic Effect and Reduction of Side Effects of Morphine in Tail-Flick and Open Field Tests (Intramuscular Administration)

### Methods.

### Tail-Flick Test.

Acute analgesic action of drugs was estimated from prolongation of the tail-flick latency in male Wistar rats (Woolf C. J. et al., 1977, Eur. J. Pharmacol., Vol. 45(3), pages 311-314; Serdyuk S. E. and Gmiro V. E., 2007, Bull. Exp. Biol. Med., Vol. 143(3), pages 350-352). In the tail-flick test, pain is stimulated by rat tail immersion in hot water at 55±0.1° C. The latent period of withdrawal of the tail (time to get rid of the pain stimulus) is determined every 3 minutes. For evaluation of pain sensitivity the rats that have short latency (3-5 seconds) during the last 15 min before administration of substances were used. Morphine, memantine, epinephrine, as well as ternary combinations of morphine with epinephrine and memantine is administered intramuscular (I.M.) in a volume of 0.1-0.3 ml in increasing doses until a maximal analgesia (measured as maximal latency of tail withdrawal in seconds) during the last three measurements of pain sensitivity. Control animals were injected I.M. 0.2 ml of distilled water. Analgesic effect is assessed by an increase of latency of tail withdrawal (s) compared to control (dist. Water).

### Open Field Test.

To evaluate the hypokinetic (sedative) or hyperkinetic action effects of tested drugs an “open field” (OF) test was applied. In the OF test, the locomotive activity of rats is determined. Animals were placed in the center of square of the illuminated field (1 meter×1 meter) for 3 minutes and the mobility time was recorded in seconds (horizontal activity). The registration of horizontal locomotive activity was performed 40 minutes after administration of the test drug(s). To quantify the locomotive activity for each dose of test drug(s) an average horizontal activity was calculated. Hypokinetic (sedative) or hyperkinetic action of morphine, memantine, epinephrine, and the triple combination of morphine with epinephrine and memantine was assessed by reduction (hypokynesia) or increase (hyperkinesia) of average horizontal activity in the OF test in % compared with measurements of rats in the control group as well as the number of rats with a significant decrease or increase the horizontal activity (50% and more as compared to the control).

TABLE 2

| Morphine | Memantine | Epinephrine | Latent period | Sedation | Morphine | Memantine | Epinephrine |
|---|---|---|---|---|---|---|---|
| (Rat mg/kg) | (Rat mg/kg) | (Rat mg/kg) | (seconds) | (% of Rats) | (mg, Human Dose) ** | (mg, Human Dose) ** | (mg, Human Dose) ** |
| | | | 4.5* | 0* | | | |
| 1 | | | 6.9 | 0 | 9.67 | | |
| 2.5 | | | 11.7 | 20 | 24.19 | | |
| 5 | | | 19.8 | 90 | 48.38 | | |
| | | 0.01 | 5 | 0 | | | 0.096 |
| | | 0.03 | 9.2 | 0 | | | 0.29 |
| | | 0.1 | 18.2 | 0 | | | 0.96 |
| | 1 | | 5.3 | 0 | | 9.67 | |
| | 3 | | 9.5 | 10*** | | 29 | |
| | 10 | | 18.7 | 90*** | | 96.77 | |
| | 1 | 0.01 | 6.5 | 0 | | 9.67 | 0.096 |
| 0.05 | 0.5 | 0.005 | 6.9 | 0 | 0.48 | 4.83 | 0.048 |
| 0.1 | 0.5 | 0.005 | 18.5 | 0 | 0.96 | 4.83 | 0.048 |
| 1 | 1 | 0.01 | 27.2 | 0 | 9.67 | 9.67 | 0.096 |
| 2.5 | 1 | 0.01 | 33.2 | 0 | 24.19 | 9.67 | 0.096 |
| 5 | 1 | 0.01 | 38.9 | 10 | 48.38 | 9.67 | 0.096 |

*N.C.—Negative control (distilled water).
** Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).
***Hyperkinesia.

### Triple-Therapy.

Combined administration of morphine in low dose of 0.05 mg/kg with epinephrine in lowest threshold dose of 0.005 mg/kg and memantine at a low threshold dose of 0.5 mg/kg insignificantly reduced sensitivity to pain compared with control, as it increased the latency of the tail-flick from 4.5 to 6.9 seconds.

Combined administration of morphine at a higher dose of 0.1 mg/kg with epinephrine lowest threshold dose of 0.005 mg/kg, and memantine at a low threshold dose of 0.5 mg/kg caused a near-maximal analgesic effect (increased the latency tail withdrawal compared to control 4.1 fold, from 4.5 to 18.5 seconds), almost the same as the high dose of morphine 5 mg/kg alone (19.8 seconds). This combination did not reduce the horizontal activity in the rats. Consequently, compositions comprising memantine in a low threshold dose of 0.5 mg/kg and epinephrine in the low threshold dose of 0.005 mg/kg reduced the effective dose of morphine by 50 times (from 5 to 0.1 mg/kg), causing maximal analgesic effect without developing sedative (hypokinetic) side effect. It is highly surprising to achieve synergy by a combined administration of 0.1 mg/kg morphine within a triple composition with epinephrine 0.005 mg/kg and memantine 0.5 mg/kg, as the threshold doses of epinephrine and memantine in the ternary composition with morphine are reduced 2 fold compared with the doses used in the composition of epinephrine 0.01 mg/kg and 1 mg/kg memantine, causing very little analgesic effect (increased the latency tail withdrawal from 4.5 to 6.5 seconds compared to control).

Combined administration of morphine in low conventional dose of 1 mg/kg with epinephrine in the threshold dose of 0.01 mg/kg and memantine in the threshold dose of 1.0 mg/kg increased the maximal analgesic effect by 1.4 fold compared to 5 mg/kg of morphine (increase the latency of tail-flick from 19.8 to 27.2 seconds) without the development of hypokinesia.

Combined morphine administration at a mean dose of 2.5 mg/kg and higher doses of 5 mg/kg with epinephrine in threshold dose of 0.01 mg/kg, and memantine in the threshold dose of 1.0 mg/kg increased the maximum analgesic effect by 1.7 and 2.0 times, respectively, compared to 5 mg/kg of morphine (increased the latency of tail withdrawal from 19.8 seconds to 33.2 and 38.9 seconds, respectively), as well as eliminated hypokinesia in 80-90% of the rats. Thus, the administration of memantine in the threshold dose of 1 mg/kg with epinephrine in threshold dose of 0.01 mg/kg in combined administration with morphine on the one hand increased the maximum effect of morphine by 1.4-2.0 times, and on the other hand reduced the effective dose of morphine by 5 times (to 1 mg/kg), causing potentiated analgesic effect without developing side hypokinetic (sedative) effect. The potentiated synergism of morphine is highly surprising when combined within a triple composition with epinephrine 0.01 mg/kg and memantine (1.0 mg/kg), comprising 1.4-2.0 fold increase in the maximum analgesic effect and a 5 fold decrease in the effective doses of morphine causing potentiated analgesic effect. The potentiation of the analgesic effect of morphine in the content of the ternary composition cannot be explained with the additional analgesic effect of the composition of epinephrine 0.01 mg/kg+memantine 1 mg/kg, as the composition of 0.01 mg/kg epinephrine+1 mg/kg memantine had very weak analgesic effect.

The results of these studies indicate that ternary compositions containing morphine, epinephrine and memantine cause analgesic effect which is significantly superior to the analgesic effect of morphine in conventional dose without developing sedative (hypokinetic) side effect, and also significantly reduce the conventional doses of morphine, memantine and epinephrine as part of composition. The above-mentioned compositions cause a potentiation of analgesic effect of morphine in a safe manner, since they eliminate the side effects of the use of each of the components of the composition.

### Example 3. Potentiation of Analgesic and Anti-Hyperalgesic Effects, and Reduction of Side Effects of Memantine, Morphine, Amitriptyline and Dipyrone in Tail-Flick, Paw Withdrawal and Open Field Tests

### Methods.

### Tail-Flick Test (Acute Pain).

Acute analgesic action of drugs was estimated from prolongation of the tail-flick latency in male Wistar rats (Woolf C. J. et al., 1977, Eur. J. Pharmacol., Vol. 45(3), pages 311-314; Serdyuk S.E. and Gmiro V.E., 2007, Bull. Exp. Biol. Med., Vol. 143(3), pages 350-352). In the tail-flick test, pain is stimulated by rat tail immersion in hot water at 55±0.1° C. The latent period of withdrawal of the tail (time to get rid of the pain stimulus) is determined every 3 minutes. For evaluation of pain sensitivity the rats that have short latency (3-5 seconds) during the last 15 minutes before administration of substances were used. Morphine, memantine, amitriptyline, dipyrone, phenylephrine, epinephrine, as well as triple combinations thereof were administered intramuscular (I.M.) or orally (intra-gavage, I.G.) in increasing doses until a maximal analgesia (measured as maximal latency of tail withdrawal in seconds) during the last three measurements of pain sensitivity. Control animals were injected I.M. 0.2 ml or orally 1.0 ml of distilled water. Analgesic effect is assessed by an increase of latency of tail withdrawal (s) compared to control (dist. Water).

### Paw Withdrawal Test (Acute Inflammatory Hyperalgesia).

Inflammatory hyperalgesia of a paw was caused by placing it into hot water (56° C.) for 20-25 seconds under the conditions of ether anesthesia. Hyperalgesia was developed 30 min after the burn (latency of paw withdrawal on its being placed into water at a temperature 47° C. was reduced from 15-20 seconds to 2-4 seconds). Acute anti-hyperalgesic action of drugs was estimated from prolongation of the paw withdrawal latency in male Wistar rats with inflammatory hyperalgesia (Coderre T. J., Melzack R. Brain Res. (1987) 404(1-2):95-106).

The latent period of paw withdrawal (time to get rid of the pain stimulus) is determined every 5 minutes. For estimation of hyperalgesia the rats that have short latency (3-5 seconds) during the last 20 min before administration of substances were used. Morphine, memantine, amitriptyline, dipyrone, phenylephrine epinephrine, as well as triple combinations thereof were administered intramuscular (I.M.) or orally in increasing doses until a maximal latency of paw withdrawal in seconds during the last three measurements of pain sensitivity. Control animals were injected I.M. 0.2 ml or orally 1.0 ml of distilled water. Anti-hyperalgesic effect is assessed by an increase of latency of paw withdrawal (s) compared to control (dist. Water).

### Open Field Test.

To evaluate the hypokinetic (sedative) or hyperkinetic action effects of tested drugs an “open field” (OF) test was applied. In the OF test, the locomotive activity of rats is determined. Animals were placed in the center of square of the illuminated field (1 meter×1 meter) for 3 minutes and the mobility time was recorded in seconds (horizontal activity). The registration of horizontal locomotive activity was performed 40 minutes after administration of the test drug(s). To quantify the locomotive activity for each dose of test drug(s) an average horizontal activity was calculated. Hypokinetic (sedative) or hyperkinetic action of morphine, memantine, epinephrine, phenylephrine, amitriptyline and dipyrone and their triple combinations were assessed by reduction (hypokinesia) or increase (hyperkinesia) of average horizontal activity in the OF test in % compared with measurements of rats in the control group as well as the number of rats with a significant decrease or increase the horizontal activity (50% and more as compared to the control).

TABLE 3

| | | | | Pow withdrawal | Hyperkinesia | |
|---|---|---|---|---|---|---|
| Morphine | Memantine | Epinephrine | Tail flick latency | latency | or sedation | Hyperalgesia |
| (Rat mg/kg) | (Rat mg/kg) | (Rat mg/kg) | (seconds) | (seconds) | (% rats) | (% rats) |
| | | | 4.5* | 4.3* | 0 | 0 |
| 0.01 | | | 1.2 | 1.3 | 0 | 90 |
| 0.02 | | | 0.9 | 1.1 | 0 | 100 |
| 0.2 | | | 4.9 | 5.2 | 0 | 0 |
| 1 | | | 6.9 | 7.2 | | 0 |
| 2.5 | | | 11.7 | 11.3 | 20** | 0 |
| 5 | | | 19.8 | 19.5 | 90** | 0 |
| | | 0.01 | 5 | 4.8 | 0 | 0 |
| | | 0.03 | 9.2 | 7.6 | 0 | 0 |
| | | 0.1 | 18.2 | 12.3 | 0 | 0 |
| | 1 | | 6.0 | 6.3 | 0 | 0 |
| | 2.5 | | 8.1 | 7.5 | 10*** | 0 |
| | 5 | | 12.7 | 12.2 | 20*** | 0 |
| | 15 | | 19.1 | 18.8 | 90*** | 0 |
| 0.01 | 1 | 0.01 | 7.4 | 7.1 | 0 | 0 |
| 0.02 | 1 | 0.01 | 19.4 | 19.1 | 0 | 0 |
| 0.5 | 1 | 0.01 | 29.5 | 29.2 | 0 | 0 |
| 5 | 1 | 0.01 | 38.9 | 37.8 | 0 | 0 |

*N.C.—Negative control (distilled water).
**Sedation in open field test.
***Hyperkinesia in open field test.

### Triple-Therapy.

Combined administration of morphine at a very low dose of 0.01 mg/kg with epinephrine in threshold dose 0.01 and memantine at a low dose of 1.0 mg/kg eliminated opioid hyperalgesia causing morphine in dose 0.01 mg/kg in tail and paw withdrawal test as it increased the latency of the tail and paw withdrawal from 0.9 and 1.1 seconds respectively to 7.4 and 7.1 seconds

Combined administration of morphine at a very low dose of 0.02 mg/kg with epinephrine in threshold dose of 0.01 and memantine in low dose 1 mg/kg not only eliminated opioid hyperalgesia causing morphine in dose 0.02 mg/kg, but also induced a maximal analgesic and anti-hyperalgesic effect reducing pain sensitivity and hyperalgesia by 4.4 and 4.5 times, compared to control (increase the latency of the tail and paw withdrawal respectively from 4.5 and 4.3 seconds to 19.4 and 19.1 seconds), such as morphine alone in high dose of 5 mg/kg. It is important to note that such a combination has not changed the horizontal activity in rats. Consequently, compositions comprising memantine in low dose 1 mg/kg and epinephrine in threshold dose 0.1 mg/kg (not effective alone) reduces the effective dose of morphine by about 250 times (from 5 to 0.02 mg/kg), causing maximal analgesic and anti-hyperalgesic effect without developing addictive (opioid hyperalgesia) and sedative side effect

It is highly surprising to achieve synergy by a combined administration of 0.02 mg/kg morphine within a triple composition with epinephrine 0.01 mg/kg and memantine 1 mg/kg, as the threshold doses of epinephrine and memantine in the ternary composition with morphine causing very little analgesic and anti-hyperalgesic effect (increased the latency tail and paw withdrawal from 4.5 and 4.3 seconds to 6.5 and 5.9 seconds compared to control).

Combined administration of morphine in low dose of 0.5 mg/kg with epinephrine in the threshold dose of 0.01 mg/kg and memantine in the threshold dose of 1.0 mg/kg increased the maximal analgesic and anti-hyperalgesic effect by 1.5 fold compared to 5 mg/kg of morphine (increase the latency of tail and paw withdrawal respectively from 19.8 and 19.5 seconds to 29.5 and 29.2 seconds) without the development of hypokinesia.

Combined morphine administration at higher doses of 5 mg/kg with epinephrine in threshold dose of 0.01 mg/kg, and memantine in the threshold dose of 1.0 mg/kg increased the maximum analgesic and hyperalgesic effect by 2.0 and 1.9 times, respectively, compared to 5 mg/kg of morphine (increased the latency of tail and paw withdrawal from 19.8 and 19.5 seconds to 38.9 and 37.8 seconds, respectively), as well as eliminated hypokinesia in 100% of the rats. Thus, the administration of memantine in the low dose of 1 mg/kg with epinephrine in threshold dose of 0.01 mg/kg in combined administration with morphine on the one hand increased the maximum effect of morphine by 1.5-2.0 times, and on the other hand reduced the effective dose of morphine by 10 times (to 0.5 mg/kg), causing potentiated analgesic and anti-hyperalgesic effect without developing side sedative effect. The potentiated synergism of morphine is highly surprising when combined within a triple composition with epinephrine 0.01 mg/kg and memantine (1.0 mg/kg), comprising 1.4-2.0 fold increase in the maximum analgesic effect and a 10 fold decrease in the effective doses of morphine causing potentiated analgesic and anti-hyperalgesic effect. The potentiation of the analgesic effect of morphine in the content of the ternary composition cannot be explained with the additional analgesic effect of the composition of epinephrine 0.01 mg/kg+memantine 1 mg/kg, as the composition of 0.01 mg/kg epinephrine+1 mg/kg memantine had very weak analgesic effect.

The results of these studies indicate that ternary compositions containing morphine, epinephrine and memantine cause analgesic and anti-hyperalgesic effect which is significantly superior to the analgesic and anti-hyperalgesic effect of morphine in conventional dose, without developing side sedative and addictive (opioid hyperalgesia) effects, and also significantly reducing the conventional doses of morphine, memantine and epinephrine as part of composition. The above-mentioned compositions cause a potentiation of analgesic effect of morphine in a safe manner, since they eliminate the side effects of the use of each of the components of the composition.

TABLE 4

| | | | | | Tail flick | Pow withdrawal | Hyperkinesia | |
|---|---|---|---|---|---|---|---|---|
| Morphine | Memantine | Phenylephrine | Amitriptyline | Dipyrone | latency | latency | or sedataion | Hyperalgesia |
| (Rat mg/kg) | (Rat mg/kg) | (Rat mg/kg) | (Rat mg/kg) | (Rat mg/kg) | (seconds) | (seconds) | (% rats) | (% rats) |
| | | | | | 4.8* | 4.5* | | |
| 0.01 | | | | | 1.3 | 1.2 | | 90 |
| 0.02 | | | | | 1.1 | 1.0 | 0 | 100 |
| 1 | | | | | 8.5 | 8.3 | 10** | 0 |
| 5 | | | | | 20.5 | 21.3 | 80** | 0 |
| | | 0.02 | | | 4.4 | 4.6 | 0 | 0 |
| | | 0.04 | | | 6.6 | 6.8 | 0 | 0 |
| | | 0.1 | | | 18.7 | 11.3 | 0 | 0 |
| | 1 | | | | 4.3 | 4.0 | 0 | 0 |
| | 5 | | | | 8.3 | 9.0 | 20*** | 0 |
| | 20 | | | | 20.3 | 21.0 | 90*** | 0 |
| | | | 0.1 | | 4.5 | 4.7 | 0 | 0 |
| | | | 0.5 | | 5.5 | 5.3 | 0 | 0 |
| | | | 2.0 | | 7.5 | 7.3 | 10** | 0 |
| | | | 10.0 | | 20.2 | 19.8 | 90** | 0 |
| | | | | 0.2 | 4.3 | 4.6 | 0 | 0 |
| | | | | 1.0 | 5.3 | 5.5 | 0 | 0 |
| | | | | 4.0 | 7.7 | 7.4 | 0 | 0 |
| | | | | 20.0 | 20.6 | 20.1 | 0 | 0 |
| 0.01 | 1 | 0.02 | | | 5.0 | 5.1 | 0 | 0 |
| 0.02 | 1 | 0.02 | | | 19.7 | 19.4 | 0 | 0 |
| 1.0 | 1 | 0.02 | | | 32.5 | 31.6 | 0 | 0 |
| 5.0 | 1 | 0.02 | | | 37.6 | 36.6 | | |
| | 1 | 0.02 | 0.05 | | 5.6 | 5.8 | 0 | 0 |
| | 1 | 0.01 | 0.1 | | 19.5 | 19.8 | 0 | 0 |
| | 1 | 0.01 | 2.0 | | 29.8 | 29.3 | 0 | 0 |
| | 1 | 0.02 | | 0.05 | 5.8 | 5.5 | 0 | 0 |
| | 1 | 0.01 | | 0.1 | 19.7 | 19.9 | 0 | 0 |
| | 1 | 0.01 | | 4.0 | 30.4 | 29.9 | 0 | 0 |

*N.C.—Negative control (distilled water).
**Sedation in open field test.
***Hyperkinesia in open field test.

### Triple-Therapy.

Combined administration of morphine at a very low dose of 0.01 mg/kg with phenylephrine in threshold dose 0.02 and memantine at a low dose of 1.0 mg/kg eliminated opioid hyperalgesia causing morphine in dose 0.01 mg/kg in tail and paw withdrawal test as it increased the latency of the tail and paw withdrawal from 1.3 and 1.2 seconds respectively to 5.0 and 5.1 seconds

Combined administration of morphine at a very low dose of 0.02 mg/kg with phenylephrine in threshold dose of 0.02 and memantine in low dose 1 mg/kg not only eliminated opioid hyperalgesia causing morphine in dose 0.02 mg/kg, but also induced a maximal analgesic and anti-hyperalgesic effect reducing pain sensitivity and hyperalgesia by 4.1 and 4.3 folds (increase the latency of the tail and paw withdrawal respectively from 4.8 and 4.5 seconds to 19.7 and 19.4 seconds), such as morphine alone in high dose of 5 mg/kg. It is important to note that such a combination has not changed the horizontal activity in rats. Consequently, compositions comprising memantine in low dose 1 mg/kg and phenylephrine in threshold dose 0.2 mg/kg (not effective alone) reduces the effective dose of morphine by about 250 times (from 5 to 0.02 mg/kg), causing maximal analgesic and anti-hyperalgesic effect without developing addictive (opioid hyperalgesia) and sedative side effect

It is highly surprising to achieve elimination of morphine (0.01-0.02 mg/kg) hyperalgesia within a triple composition with phenylephrine 0.02 mg/kg and memantine 1 mg/kg. It is highly surprising to achieve synergy by a combined administration of 0.02 mg/kg morphine within a triple composition with phenylephrine 0.02 mg/kg and memantine 1 mg/kg, as the threshold doses of phenylephrine and memantine in the ternary composition with morphine causing very little analgesic and anti-hyperalgesic effect (increased the latency tail and paw withdrawal from 4.8 and 4.5 s to 5.2 and 5.4 seconds compared to control).

Combined administration of morphine in low conventional dose of 1 mg/kg with phenylephrine in the threshold dose of 0.02 mg/kg and memantine in the low dose of 1.0 mg/kg increased the maximal analgesic and anti-hyperalgesic effect by 1.6 and 1.5 times compared to 5 mg/kg of morphine (increased latency of tail and paw withdrawal from 20.5 and 21.3 seconds respectively to 32.5 and 31.6 seconds), as well as eliminated sedation in 100% of the rats.

Combined morphine administration at higher doses of 5 mg/kg with phenylephrine in threshold dose of 0.02 mg/kg, and memantine in the low dose of 1.0 mg/kg increased the maximum analgesic and hyperalgesic effect by 1.8 and 1.7 times, compared to 5 mg/kg of morphine (increased the latency of tail and paw withdrawal from 20.5 and 21.3 seconds to 37.6 and 36.6 seconds, respectively), as well as eliminated hypokinesia in 100% of the rats. Thus, the administration of memantine in the low dose of 1 mg/kg with phenylephrine in threshold dose of 0.02 mg/kg in combined administration with morphine on the one hand increased the maximum effect of morphine by 1.5-1.8 times, and on the other hand reduced the effective dose of morphine by 5 times (to 1 mg/kg), causing potentiated analgesic and anti-hyperalgesic effect without developing side hypokinetic (sedative) effect. The potentiated synergism of morphine is highly surprising when combined within a triple composition with phenylephrine 0.02 mg/kg and memantine (1.0 mg/kg), comprising 1.5-1.8 fold increase in the maximum analgesic and anti-hyperalgesic effect and a 5 fold decrease in the effective doses of morphine causing potentiated analgesic and anti-hyperalgesic effect. The potentiation of the analgesic and anti-hyperalgesic effect of morphine in the content of the ternary composition cannot be explained with the additional analgesic effect of the composition of phenylephrine 0.02 mg/kg+memantine 1 mg/kg, as the composition of 0.02 mg/kg phenylephrine+1 mg/kg memantine had very weak analgesic and anti-hyperalgesic effect.

Combined administration of amitriptyline at a very low dose of 0.05 mg/kg with phenylephrine in threshold dose of 0.02 and memantine in low dose 1 mg/kg practically did not change the sensitivity to pain and hyperalgesia (the latency of the tail and paw withdrawal changed from 4.8 and 4.5 seconds to 5.6 and 5.8 seconds, respectively).

Combined administration of amitriptyline at a low dose of 0.1 mg/kg with phenylephrine in threshold dose of 0.02 and memantine in low dose 1 mg/kg induced a maximal analgesic and anti-hyperalgesic effect reducing pain sensitivity and hyperalgesia by 4.2 and 4.4 folds, (increase the latency of the tail and paw withdrawal respectively from 4.8 and 4.5 seconds to 19.5 and 19.8 seconds), such as amitriptyline alone in high dose of 10 mg/kg. It is important to note that such a combination has not changed the horizontal activity in rats. Consequently, compositions comprising memantine in low dose 1 mg/kg and phenylephrine in threshold dose 0.2 mg/kg (not effective alone) reduces the effective dose of amitriptyline by about 100 times (from 10 to 0.1 mg/kg), causing maximal analgesic and anti-hyperalgesic effect without developing sedative side effect

Combined administration of amitriptyline in low conventional dose of 2 mg/kg with phenylephrine in the threshold dose of 0.02 mg/kg and memantine in the low dose of 1.0 mg/kg increased the maximal analgesic and anti-hyperalgesic effect by 1.5 and 1.5 times compared to 10 mg/kg of amitriptyline (increased latency of tail and paw withdrawal from 20.2 and 19.8 seconds respectively to 29.8 and 29.3 seconds), as well as eliminated sedation in 100% of the rats.

Thus, the administration of memantine in the low dose of 1 mg/kg with phenylephrine in threshold dose of 0.02 mg/kg in combined administration with amitriptyline on the one hand increased the maximum effect of amitriptyline by 1.5 time, and on the other hand reduced the effective dose of amitriptyline by 5 times (to 2 mg/kg), causing potentiated analgesic and anti-hyperalgesic effect without developing side hypokinetic (sedative) effect. The potentiated synergism of amitriptyline is highly surprising when combined within a triple composition with phenylephrine 0.02 mg/kg and memantine (1.0 mg/kg), comprising 1.5 fold increase in the maximum analgesic and anti-hyperalgesic effect and a 5 fold decrease in the effective doses of amitriptyline causing potentiated analgesic and anti-hyperalgesic effect. The potentiation of the analgesic and anti-hyperalgesic effect of amitriptyline in the content of the ternary composition cannot be explained with the additional analgesic effect of the composition of phenylephrine 0.02 mg/kg+memantine 1 mg/kg, as the composition of 0.02 mg/kg phenylephrine+1 mg/kg memantine had very weak analgesic and anti-hyperalgesic effect.

Combined administration of dipyrone at a very low dose of 0.05 mg/kg with phenylephrine in threshold dose of 0.02 and memantine in low dose 1 mg/kg practically did not change the sensitivity to pain and hyperalgesia (the latency of the tail and paw withdrawal changed from 4.8 and 4.5 seconds to 5.8 and 5.5 seconds, respectively).

Combined administration of dipyrone at a low dose of 0.1 mg/kg with phenylephrine in threshold dose of 0.02 and memantine in low dose 1 mg/kg induced a maximal analgesic and anti-hyperalgesic effect reducing pain sensitivity and hyperalgesia by 4.1 and 4.4 folds (increase the latency of the tail and paw withdrawal respectively from 4.8 and 4.5 seconds to 19.7 and 19.9 seconds), such as dipyrone alone in high dose of 20 mg/kg. It is important to note that such a combination has not changed the horizontal activity in rats. Consequently, compositions comprising memantine in low dose 1 mg/kg and phenylephrine in threshold dose 0.2 mg/kg (not effective alone) reduces the effective dose of dipyrone by about 200 times (from 20 to 0.1 mg/kg), causing maximal analgesic and anti-hyperalgesic effect without developing side locomotor effect

Combined administration of dipyrone in low conventional dose of 4 mg/kg with phenylephrine in the threshold dose of 0.02 mg/kg and memantine in the low dose of 1.0 mg/kg increased the maximal analgesic and anti-hyperalgesic effect by 1.5 and 1.5 times compared to 20 mg/kg of dipyrone (increased latency of tail and paw withdrawal from 20.6 and 20.1 seconds respectively to 30.4 and 29.9 seconds)

Thus, the administration of memantine in the low dose of 1 mg/kg with phenylephrine in threshold dose of 0.02 mg/kg in combined administration with dipyrone on the one hand increased the maximum effect of dipyrone by 1.5 time, and on the other hand reduced the effective dose of dipyrone by 5 times (to 4 mg/kg), causing potentiated analgesic and anti-hyperalgesic effect without developing side locomotor effect. The potentiated synergism of dipyrone is highly surprising when combined within a triple composition with phenylephrine 0.02 mg/kg and memantine (1.0 mg/kg), comprising 1.5 fold increase in the maximum analgesic and anti-hyperalgesic effect and a 5 fold decrease in the effective doses of dipyrone causing potentiated analgesic and anti-hyperalgesic effect. The potentiation of the analgesic and anti-hyperalgesic effect of dipyrone in the content of the ternary composition cannot be explained with the additional analgesic effect of the composition of phenylephrine 0.02 mg/kg+memantine 1 mg/kg, as the composition of 0.02 mg/kg phenylephrine+1 mg/kg memantine had very weak analgesic and anti-hyperalgesic effect.

The results of these studies indicate that ternary compositions containing morphine, phenylephrine and memantine, amitriptyline, phenylephrine and memantine, and dipyrone, phenylephrine and memantine cause analgesic and anti-hyperalgesic effect which is significantly superior to the analgesic and anti-hyperalgesic effect of morphine, amitriptyline and dipyrone alone in conventional doses, without developing sedative side effect, and also significantly reduce the conventional doses of morphine, amitriptyline, dipyrone, memantine and phenylephrine as part of composition. The above-mentioned compositions cause a potentiation of analgesic and anti-hyperalgesic effect of morphine in a safe manner without development opioid hyperalgesia and sedative side effects.

### Example 4. Potentiation of the Anti-Parkinsonian Effect and Reduction of the Side Effects of Levodopa in a Haloperidol Catalepsy Model (Oral Administration)

### Methods.

Anti-parkinsonian effect of drugs was investigated on a model of induced catalepsy by I.M. injection of haloperidol at a dose of 1 mg/kg in Wistar rats (weight 160-170 g). Catalepsy was evaluated by duration of immobilization of rats positioned on a large grid fixed at an angle of 45 degrees 60 minutes after the haloperidol administration. Maximum catalepsy in rats was recorded in the case of their complete immobilization on grid after 180 seconds of observation (Campbell A., et al., 1988, Neuropharmacology, Vol. 27(11), pages 1197-1199; Ossowska K. J., Neural. Transm. Park. Dis. Dement. Sect., 1994, Vol. 8(1-2), pages 39-71; Gmiro V.E. and Serdyuk S.E., Bulletin of Experimental Biology and Medicine, 2007, Vol. 143 (5), pages 554-556).

Levodopa, memantine, phenylephrine, as well as combinations thereof were administered orally in a volume of 1.0 ml through a rigid metal probe, 45 minutes before the administration of haloperidol. Control animals received orally 1 ml of distilled water.

Anti-parkinsonian drugs effects were estimated as decrease of the average duration of immobilization of animals treated by test compositions compared to a control (DW).

### Open Field Test.

To evaluate the (hyperkinetic) side effects of tested drugs, an “open field” (OF) test was applied. In the OF test, the locomotive activity of rats is determined Animals were placed in the center of square of the illuminated field (1 meter×1 meter) for 3 minutes and the mobility time was recorded in seconds (horizontal activity). The registration of horizontal locomotive activity was performed 40 minutes after administration of the test drug(s), 5 minutes before the administration of haloperidol. To quantify the locomotive activity for each dose of test drug(s) an average horizontal activity was calculated. Hyperkinetic effect of levodopa, phenylephrine, memantine, as well as of combinations phenylephrine with memantine and levodopa was evaluated by an average increase in horizontal activity in the OF test in % compared with measures for rats in the control group, and also as the number of rats with significant increase in horizontal activity (50% and more as compared to control).

Intramuscular (IM) injection of haloperidol at a dose of 1 mg/kg, 60 minutes after injection, results in the control group in immobilization of 158.2±25 seconds (Table 5).

TABLE 5

| Levodopa | Memantine | Phenylephrine | Time on grid | Hyperkinesia | Levodopa | Memantine | Phenylephrine |
|---|---|---|---|---|---|---|---|
| (Rat mg/kg) | (Rat mg/kg) | (Rat mg/kg) | (seconds) | (% of Rats) | (mg, Human Dose) ** | (mg, Human Dose) ** | (mg, Human Dose) ** |
| | | | 158.2* | 0* | | | |
| 1 | | | 147 | 0 | 9.6 | | |
| 3 | | | 125 | 0 | 29 | | |
| 10 | | | 85 | 50 | 96.7 | | |
| 20 | | | 35 | 100 | 193.5 | | |
| | | 0.1 | 137 | 0 | | | 0.9 |
| | | 0.3 | 98 | 0 | | | 2.9 |
| | | 1 | 78 | 0 | | | 9.6 |
| | 1 | | 139 | 0 | | 9.6 | |
| | 3 | | 108 | 0 | | 29 | |
| | 10 | | 65 | 50 | | 96.7 | |
| | 20 | | 34 | 100 | | 193.5 | |
| 0.25 | 1 | 0.1 | 81 | 0 | | | |
| 0.5 | 1 | 0.1 | 28 | 0 | 4.83 | 9.67 | 0.96 |
| 3 | 1 | 0.1 | 12 | 0 | 29.03 | 9.67 | 0.96 |
| 10 | 1 | 0.1 | 6 | 0 | 96.77 | 9.67 | 0.96 |
| 10 | 3 | 0.1 | 5 | 0 | 96.77 | 29.03 | 0.96 |
| 20 | 1 | 0.1 | 3 | 0 | 193.54 | 29.67 | 0.96 |

*N.C.—Negative control (distilled water).
**Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).

### Triple-Therapy.

Combined administration of levodopa 0.25 mg/kg with memantine in low dose of 1 mg/kg and phenylephrine at a threshold dose 0.1 mg/kg caused a mild but significant anti-parkinsonian effect, as it reduced haloperidol catalepsy by a factor of 2.0 (reduces the duration of immobilization on the grid from 158 to 81 seconds)

Combined levodopa administration in a dose of 1.0 mg/kg with memantine in the low dose of 1 mg/kg and phenylephrine at a threshold dose of 0.1 mg/kg caused a significant, nearly maximal anti-parkinsonian effect (reduced the duration of immobilization on the grid by a factor of 4, from 158 to 28 seconds), comparable to levodopa and memantine in the high dose of 20 mg/kg for independent use. This combination has not increased the horizontal activity of rats. Consequently, compositions comprising memantine in a low dose of 0.1 mg/kg and phenylephrine in a threshold dose 0.1 mg/kg reduced the effective dose of levodopa causing maximal anti-parkinsonian effect without developing hyperkinetic side effect by 40 times (20.0 mg/kg versus 0.5 mg/kg).

Combined administration of levodopa in conventional low dose of 3 mg/kg with memantine in the threshold dose of 1 mg/kg and phenylephrine at a threshold dose of 0.1 mg/kg leads to a 2.8 fold increases in the maximal anti-parkinsonian effect compared to levodopa at a dose of 20 mg/kg (reduced immobilization duration the grid from 35 to 12 seconds), without development hyperkinesia in rats. In comparison, the composition of memantine in the threshold dose 1 mg/kg with phenylephrine at a threshold dose of 0.1 mg/kg caused only a mild anti-parkinsonian effect, as it reduced the amount of haloperidol catalepsy from 158 to 79 seconds, while in combining with oral administration of levodopa on the one hand multiplies the maximal anti-parkinsonian effect of levodopa, and on the other hand reduced by 7 times (to 3 mg/kg) the effective dose of levodopa causing potentiated anti-parkinsonian effect without developing side hyperkinetic effects. It is highly surprising that the potentiated synergy upon combined administration of levodopa within a triple composition with phenylephrine 0.1 mg/kg and memantine 1.0 mg/kg, results in a multiple increase in anti-parkinsonian action and 7 fold reduction of the effective dose of levodopa causing potentiated effect.

Combined administration of levodopa in medium therapeutic dose of 10 mg/kg and in the highest dose of 20 mg/kg with memantine in threshold dose of 1 mg/kg and phenylephrine at a threshold dose of 0.1 mg/kg increased the maximum anti-parkinsonian effect by a factor of 5-8 compared to levodopa alone in a dose of 20 mg/kg (immobilization duration on the grid decreases from 35 to 6 and 3 seconds, respectively) and also eliminate hyperkinesia in all rats.

The results of these studies indicate that ternary compositions containing levodopa, phenylephrine, and memantine exert anti-parkinsonian effect which is vastly superior to the anti-parkinsonian effect of levodopa in the conventional dose, without developing hyperkinetic side effect and significantly reducing the dosage of conventional levodopa, memantine and phenylephrine in the composition. The above-mentioned compositions cause a potentiation of the anti-parkinsonian action of levodopa in a safe manner, since it eliminates the side effects of the use of each of the components of the composition.

### Example 5. Potentiation of the Anti-Parkinsonian and Neuro-Protective Effects, and Elimination of Side Effects, of Levodopa and Memantine in Rotenone-Induced Parkinson Disease (PD) and Progressive Supranuclear Palsy (PSP) in Rats (Oral Administration)

Rotenone is a specific inhibitor of mitochondrial complex 1, which causes oxidative stress and apoptotic death of dopaminergic and cholinergic neurons in the striatum, substantia nigra and cerebellum. Damage to these neurons causes a decrease in dopamine and acetylcholine levels in the striatum, extrapyramidal disorders (catalepsy, impaired coordination of movements) and oligokinesia typical for PD, as well as ataxia, limb dystonia and mortality characteristic of PSP. Rotenone is used to create a model of PD and PSP in Wistar rats by intraperitoneal introduction of rotenone in a toxic dose of 2.3 mg/kg (Jason R. Cannon et al., Neurobiology of Disease, 2009, Vol. 34, pages 279-290). This model was used to conduct testes and comparisons of anti-parkinsonian and neuro-protective activity of levodopa and the combination of phenylephrine with memantine and levodopa after chronic oral administration at prophylactic scheme (Yang Y. et al., J. Neurosci. Res., 2005, Vol. 80 (3), pages 442-449; Cannon J.R. et al., Neurobiol. Dis., 2009, Vol. 34 (2), pages 279-290).

### Administration of Rotenone.

Rotenone was dissolved in mixture of DMSO and Miglyol 812N at a ratio of 2:98 at concentration of 2.3 mg/ml. Preparation: 230 mg sample of rotenone in vials was dissolved in 2 ml of DMSO. The solution was poured into a jar with 100 ml. Vials are rinsed twice with 5 ml of Miglyol, washings attached to the main solution and stirred. To this solution was added 88 ml of Miglyol and mixed again. The resulting solution was divided into 3 doses. The solution was stored at −10° C. Each animal was administered 0.2 ml of a solution of rotenone: a dose of 0.46 mg per animal weighing 200±20 g, i.e. 2.3 mg/kg. The solution of rotenone was injected IP one time a day for 19 days.

Anti-parkinsonian effect of therapy in rats with rotenone-induced PD was estimated according to elimination of extrapyramidal disorders (catalepsy) and oligokinesia (rapid decreased amplitude and velocity of repeated movements) in the “open field” test. The neuro-protective effect of therapy was evaluated by the elimination of ataxia limb dystonia and mortality in rats with rotenone-induced PSP. Hyperkinetic side action (hyper-locomotion) was evaluated by increased horizontal and vertical activity in the open field at the 10th and 18th day of the experiment, calculated in % compared to the first day of the experiment.

Phenylephrine 0.3 mg/kg, levodopa (10 and 20 mg/kg), memantine 2.5 and 5 mg/kg, combination of memantine 1 mg/kg+phenylephrine 0.3 mg/kg+levodopa 5 mg/kg, combination of memantine 1 mg/kg+phenylephrine 0.3 mg/kg+levodopa 10 mg/kg and DW (1.0 ml, control) were orally administered using rigid metal probe in volumes of 1 ml daily for 19 days, 45 minutes before administration of rotenone. The number of animals in the control and experimental groups ranged from 6 to 8.

Levodopa was administered in combination with benserazide (peripheral dopa-decarboxylase inhibitor) at a ratio of 4:1 to reduce the side effects of levodopa, associated with the stimulation of peripheral dopamine receptors (Alam M. et al., Behav. Brain Res., 2004, Vol. 153 (2), pages 439-446). Levodopa with benserazide were dissolved in 1 ml of DW and administered orally in the volume of 1 ml. Combination of levodopa with phenylephrine, of levodopa and of benserazide solution (0.5 ml) was mixed with 0.5 ml of phenylephrine solution. Combination of levodopa with memantine and phenylephrine of levodopa and of benserazide solution (0.5 ml) was mixed with 0.5 ml of phenylephrine+memantine solution. The mixture was administered orally in a volume of 1 ml. Anti-parkinsonian effect in the experimental groups of rats with rotenone has been evaluated by eliminating catalepsy and oligokinesia.

Catalepsy in rats is determined by the time of immobilization of the animal, placed on a large grid predisposed at an angle of 45°. Maximum catalepsy in rats is recorded in the case of their complete immobilization on grid at 120 seconds for observation. The value of catalepsy is evaluated in points: 3 points—immobilization duration from 80 seconds to 120, 2 points—immobilization duration from 40 to 70, 1 point—immobilization duration from 20 to 35, 0 points—the immobilization of less than 20 seconds. Catalepsy was followed in rats daily, 30 minutes before administration of rotenone and 180 minutes after administration of rotenone. For each experimental group, on a daily basis, throughout the period of observation number of rats with severe catalepsy (2-3 points) was determined in % of the total number of rats in group. To assess the anti-cataleptic effects of therapy, on 12th and 19th day of experiment, reduction in the number of rats with severe catalepsy (2-3 points) in % was determined compared to control treated with distilled water.

Locomotive activity of rats was tested in the “open field” test. Animals were placed in the center of square of the illuminated field (1 meter×1 meter) and during 3 minutes the distance of movement (horizontal activity) and the number of rearing (vertical activity) was recorded. Registration of horizontal and vertical locomotive activity was carried out in first day for rats with high selection locomotive activity (total walking time with not less than 12 seconds and the number of vertical uprights at least 4), 60 minutes before the first administration of rotenone. To identify the rotenone-induced oligokinesia in each experimental group the locomotive activity in the open field test was examined again 2 hours after the administration of rotenone on days 12 and 19 of the experiment.

Quantification of oligokinesia in points: 0 points—the highest horizontal activity (walking total time more than 12 seconds) and high vertical activity (number of vertical posts) more than 3; 1 point—reducing the horizontal activity (total walking time 7-11 seconds) and a decrease in vertical activity (the number of vertical columns 1-3); 2 points—a significant reduction in horizontal activity (total walking time 2-6 seconds) and the absence of vertical activity (the number of vertical columns 0-1); 3 points—the absence of horizontal activity (total walking time 0-1 seconds) and the absence of vertical activity (number of vertical columns 0). In each experimental group 120 minutes after administration of rotenone on days 12 and 19 of experiment the number of rats with severe oligokinesia (2-3 points) in % of the total number of rats in group was determined.

The neuro-protective effect in the experimental groups of rats with rotenone-induced PSP was evaluated due the elimination of ataxia, limb dystonia and mortality caused by cerebellar neurotoxic action of rotenone (J. Neurochem., 2005, Vol. 95(4):930-9).

Ataxia and limb dystonia is typical incoordination neurotoxic effect of rotenone manifested in uncoordinated movements and hyper-locomotion, replaced by hypokinesia and stereotypes, and in severe cases a complete violation of antigravity reflexes, falling on its side dystonic posture and akinesia. Assessment of the severity of ataxia and limb dystonia in scores was performed using the following scale: (+) uncoordinated movement, hyperkinesia; (++) significant incoordination, hypokinesia, stereotypes, falling on its side; frequent spontaneous dystonic postures (+++) complete violation of antigravity reflexes, akinesia, sustained dystonic posture (J. Neurosci., 2014, 27; 34(35):11723-32)

Ataxia and limb dystonia registration was performed twice daily, 30 minutes before administration of rotenone and 180 minutes after administration of rotenone. For each experimental group on a daily basis, throughout the whole period of observation number of rats with severe ataxia and limb dystonia (++−+++) and mortality in % of the total number of rats in group was determined. The neuro-protective effect of therapy (effects on rate of ataxia, limb dystonia and lethality) in rats with PSP was determined at days 12 and 19 of the experiment as reducing the number of rats with severe ataxia limb dystonia and mortality in % compared to control. Side hyperkinetic effects were evaluated in the “open field” test. In this test the locomotive activity in rats is determined. Animals were placed in the center of square of the illuminated field (1 meter×1 meter) for 3 min total time recorded in seconds distance (horizontal activity) and the number of rearing (vertical activity). Registration of horizontal and vertical locomotive activity in OF assay was performed on the 10th and 18th day of the experiment, within 40 minutes after administration of the substance and 5 minutes before administration of rotenone. Side hyperkinetic effects was evaluated by the number of rats with a significant (at least 50% compared with the first day of the experiment) increase in horizontal and vertical activity (hyperkinesia) as a % of the total number of rats in group.

TABLE 6
Potentiation of the anti-parkinsonian and neuro-protective effects, and elimination
of side effects of levodopa¹ and memantine in rotenone-induced PD and PSP in rats.

| | | | | | | | Severe ataxia | | | | | | |
|---|---|---|---|---|---|---|---|---|---|---|---|---|---|
| | | | | | | | sustained dystonic | | | | | | |
| Doses for rat | | | Severe | | Severe | | postures and lethality | | | | Doses for human | | |
| (mg/kg) | | | catalepsy* | | oligokinesia** | | (% of group)*** | | Hyperkinesia**** | | (mg) | | |
| | Phenyl- | Mem- | (% of group) | | (% of group) | | [lethality] | | (% of group) | | | Phenyl- | Mem- |
| Levodopa | ephrine | antine | Day 12 | Day 19 | Day 12 | Day 19 | Day 12 | Day 19 | Day 10 | Day 18 | Levodopa² | ephrine² | antine² |
| | | | 42³ | 58³ | 100³ | 100³ | 14[0]³ | 42[14]³ | 0³ | 0³ | | | |
| | | 2.5 | 33 | 50 | 83 | 100 | 17[17] | 34[17] | 0 | 0 | | | 24 |
| | | 5 | 17 | 17 | 50 | 50 | 0[17] | 17[17] | 40 | 60 | | | 48 |
| | 0.3 | | 28 | 42 | 86 | 100 | 14[0] | 28[14] | 0 | 0 | | 2.9 | |
| 10 | | | 28 | 42 | 86 | 100 | 14[0] | 42[14] | 0 | 0 | 96.7 | | |
| 20 | | | 12 | 24 | 50 | 63 | 0[0] | 24[0] | 88 | 75 | 193.5 | | |
| 5 | 0.3 | 1 | 12 | 12 | 12 | 24 | 0 | 0 | 0 | 0 | 48.3 | 2.9 | 9.6 |
| 10 | 0.3 | 1 | 0 | 0 | 0 | 12 | 0 | 0 | 0 | 0 | 96.7 | 2.9 | 9.6 |

¹Oral administration 45 minutes before the administration of rotenone 2.3 mg/kg in duration 19 days.
²Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).
³N.C.—Negative control (distilled water).
*The immobilization time of a rat on an inclined grid above 40 seconds.
**Oligokinesia ++-+++ in OF test.
***Ataxia and limb dystonia ++-+++.
****Increased locomotive activity in OF test more than 50% compared to the first day of experiment.

### Triple-Therapy.

Combined oral administration of levodopa in low conventional dose 5 mg/kg with memantine in low dose of 1 mg/kg and phenylephrine at a low dose 0.3 mg/kg on days 12 and 19 of experiment had reduced the number of rats with severe oligokinesia by 88% and 76% compared to control (from 100% to 12% and 24%) indicating a very significant reduction of oligokinesia in rats with rotenone-induced PD. This combination on days 12 and 19 of the experiment reduced the number of rats with severe catalepsy with maximum efficiency to 30% and 46% respectively compared to control (from 42% to 12% and from 58% to 12%). A combination of levodopa 5 mg/kg with memantine 1 mg/kg and phenylephrine 0.3 mg/kg full eliminated ataxia, limbic dystonia and mortality on days 12 and 19 of the experiment. Aforementioned combination did not cause hyperkinesia in rats in the OF test on the 10th and 18th day of experiment. Thus, the combination of levodopa in low dose 5 mg/kg with memantine in low dose 1 mg/kg) and phenylephrine in the low dose of 0.3 mg/kg caused maximal antiparkinsonian and neuroprotective effect, which is vastly superior to the efficacy of levodopa in maximal dose 20 mg/kg alone. The advantage of the aforementioned combination is a complete absence of hyperkinetic side effect in rats.

Combined oral administration of levodopa in middle conventional dose 10 mg/kg with memantine in low dose of 1 mg/kg and phenylephrine at a low dose 0.3 mg/kg on days 12 and 19 of experiment had reduced the number of rats with severe oligokinesia by 100% and 88% compared to control (from 100% to 0% and 12%) indicating almost full elimination of oligokinesia in rats with rotenone-induced PD. This combination on days 12 and 19 of the experiment full eliminated severe catalepsy ataxia, limbic dystonia and mortality. Aforementioned combination did not cause hyperkinesia in rats in the OF test on the 10th and 18th day of experiment. Thus, the combination of levodopa in middle conventional dose 10 mg/kg with memantine in low dose 1 mg/kg) and phenylephrine in the low dose of 0.3 mg/kg caused maximal possible antiparkinsonian and neuroprotective effect, which on 50-100% superior efficacy of levodopa in maximal dose 20 mg/kg alone.The advantage of the aforementioned combination is a complete absence of hyperkinetic side effect in rats.

It is highly surprising that the potentiated synergy upon combined administration of levodopa within a triple composition with phenylephrine 0.3 mg/kg and memantine 1.0 mg/kg, results in a multiple increase in anti-parkinsonian and neuroprotective action and 2-4 fold reduction of the effective dose of levodopa (20 mg/kg).

Therefore, phenylephrine that is not effective alone in a dose of 0.3 mg/kg and memantine that is not effective alone in a dose of 1 mg/kg synergistically potentiated the anti-parkinsonian and neuro-protective effects of levodopa in a low dose of 5-10 mg/kg that had low efficacy while being used alone, to the maximal level, which cannot be explained in view of the independent use of levodopa in the high dose of 20 mg/kg alone. However, phenylephrine and memantine did not enhance the hyperkinetic side effect of levodopa at dose of 5-10 mg/kg.

The results of these studies indicate that ternary compositions containing levodopa, phenylephrine, and memantine exert anti-parkinsonian and neuroprotective effect which is vastly superior to the anti-parkinsonian and neuroprotective effect of levodopa in the conventional dose, without developing hyperkinetic side effect and significantly reducing the dosage of conventional levodopa, memantine and phenylephrine in the composition. The above-mentioned compositions cause a potentiation of the anti-parkinsonian and neuroprotective action of levodopa in a safe manner, since it eliminates the side effects of the use of each of the components of the composition.

According to the results of the experiments, the combination of levodopa with phenylephrine and memantine in conventional low doses can be offered for the safe treatment of severe Parkinson's disease and supranuclear progressive palsy, resistant to the action of levodopa, as well as for the treatment of Parkinson's disease and PSP in patients who cannot tolerate levodopa.

### Example 6. Potentiation of the Anti-Parkinsonian Effect and Reduction of the Side Effects of Levodopa by Phenylephrine in a Haloperidol Catalepsy Model (Oral Administration)

### Methods.

Anti-parkinsonian effect of drugs was investigated on a model of induced catalepsy by I.M. injection of haloperidol at a dose of 1 mg/kg in Wistar rats (weight 160-170 g). Catalepsy was evaluated by duration of immobilization of rats positioned on a large grid fixed at an angle of 45 degrees 60 minutes after the haloperidol administration. Maximum catalepsy in rats was recorded in the case of their complete immobilization on grid after 180 sec of observation (Campbell A., et al., 1988, Neuropharmacology, Vol. 27(11), pages 1197-1199; Ossowska K. J., Neural. Transm. Park. Dis. Dement. Sect., 1994, Vol. 8(1-2), pages 39-71; Gmiro V.E. and Serdyuk S.E., Bulletin of Experimental Biology and Medicine, 2007, Vol. 143 (5), pages 554-556).

Levodopa and phenylephrine were administered orally in a volume of 1.0 ml through a rigid metal probe, 45 minutes before the administration of haloperidol. Control animals received orally 1 ml of distilled water.

Anti-parkinsonian agents effects were estimated as decrease of the average duration of immobilization of animals treated by test compositions compared to a control (DW).

### Open Field Test.

To evaluate the side hyperkinetic effects of drugs, the test an “open field” (OF) test was applied. In the OF test, the locomotive activity of rats is determined Animals were placed in the center of square of the illuminated field (1 meter×1 meter) for 3 minutes and the mobility time was recorded in seconds (horizontal activity). The registration of horizontal locomotive activity was performed 40 minutes after administration of the test drug(s), 5 minutes before the administration of haloperidol. To quantify the locomotive activity for each dose of the test drug(s), an average horizontal activity was calculated. Hyperkinetic effect of levodopa, phenylephrine, as well as of combinations of levodopa and phenylephrine were evaluated by an average increase in horizontal activity in the OF test in % compared with measures for rats in the control group, and also as the number of rats with significant increase in horizontal activity (50% and more as compared to control).

Intramuscular (IM) injection of haloperidol at a dose of 1 mg/kg, 60 minutes after injection, results in the control group in immobilization of 158.2±25 seconds (Table 7).

TABLE 7

| Levodopa | Phenylephrine | Time on grid | Hyperkinesia | Levodopa | Phenylephrine |
|---|---|---|---|---|---|
| (Rat mg/kg) | (Rat mg/kg) | (seconds) | (% of Rats) | (mg, Human Dose)** | (mg, Human Dose)** |
| | | 158.2* | 0* | | |
| 1 | | 147 | 0 | 9.6 | |
| 3 | | 125 | 0 | 29 | |
| 10 | | 85 | 50 | 96.7 | |
| 20 | | 35 | 100 | 193.5 | |
| | 0.1 | 137 | 0 | | 0.9 |
| | 0.3 | 98 | 0 | | 2.9 |
| | 1 | 78 | 0 | | 9.6 |
| 1.5 | 0.1 | 75 | 0 | 14.5 | 0.9 |
| 3 | 0.1 | 37 | 0 | 29 | 0.9 |
| 3 | 0.3 | 33 | 0 | 29 | 2.9 |
| 10 | 0.3 | 17 | 0 | 96.7 | 2.9 |
| 20 | 0.3 | 10 | 0 | 193.5 | 2.9 |
| 20 | 0.1 | 29 | 40 | 193.5 | 0.9 |

*N.C.—Negative control (distilled water).
**Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).

### Dual-Therapy.

Combined administration of levodopa in low dose of 1.5 mg/kg and phenylephrine at a threshold dose (0.1 mg/kg, which was ineffective independently) caused a mild but significant anti-parkinsonian effect, as it reduced haloperidol catalepsy by a factor of 2.1 (reduces the duration of immobilization on the grid from 158 to 75 seconds). Combined levodopa administration in a low dose of 3 mg/kg with phenylephrine at a threshold dose of 0.1 mg/kg caused a significant anti-parkinsonian effect (reduced the duration of immobilization on a grid by a factor of 4.3, from 158 to 37 seconds), similar to levodopa in the high dose of 20 mg/kg alone. It is important to note that such a combination has not increased the horizontal activity in rats. Therefore, a threshold dose of phenylephrine (0.1 mg/kg) reduces the effective dose of levodopa by about 7 times, achieving the maximal effect without developing side effects.

Combined levodopa administration in doses of 3, 10 and 20 mg/kg with phenylephrine at a dose of 0.3 mg/kg had a maximal anti-parkinsonian effect (reduced the duration of immobilization on the grid from 158 to 33, 17 and 10 seconds, respectively), which is 1.2, 2 and 3.5 times better than levodopa in the high dose of 20 mg/kg alone. It is important to note that such combinations had also not increased the horizontal activity in rats. Therefore, a dose of phenylephrine (0.3 mg/kg) reduced the effective dose of levodopa by 2 to 7 times, reaching maximal effect without developing side effects.

Combined levodopa administration in low dose (3 mg/kg) with phenylephrine in conventional dose of 0.3 mg/kg did not substantially increase the anti-parkinsonian effect as compared with the combination of levodopa at a dose of 3 mg/kg and phenylephrine at a threshold dose of 0.1 mg/kg (immobilization time on grid, 33 and 37 seconds, respectively). Thus, in order to reduce the effective dose of levodopa it is sufficient to use a threshold dose of phenylephrine (0.1 mg/kg), but for reaching the maximal anti-parkinsonian action of levodopa, higher conventional dose of phenylephrine (0.3 mg kg) should be used.

Combined administration of levodopa in a high dose (20 mg/kg) with phenylephrine at a threshold dose (0.1 mg/kg) did not significantly (a factor of 1.2) increase the maximal anti-parkinsonian effect of levodopa alone (reducing the duration of immobilization on the grid from 35 to 29 seconds) but eliminated hyperkinesia in 60% of the rats. Combined administration of levodopa in medium therapeutic dose (10 mg/kg) and in high therapeutic dose (20 mg/kg) with phenylephrine conventional medium dose (0.3 mg/kg, had small anti-parkinsonian effect alone) had increased the maximal anti-parkinsonian effect of levodopa in a dose of 20 mg/kg by 2.0 and 3.5 fold, respectively (immobilization duration decreases from 35 to 17 and 10 seconds, respectively), and also eliminated hyperkinesia in 100% of rats. Thus, phenylephrine in conventional average dose of 0.3 mg/kg significantly increases the maximum anti-parkinsonian effect of levodopa in conventional doses without developing hyperkinetic side effect.

The potentiated synergism of orally administered combination of levodopa with conventional dose of phenylephrine (0.3 mg/kg) is highly surprising, comprising a 2.0-3.5 fold increase in the maximal antiparkinsonian effect and a 2 fold decrease in the convention's dose of levodopa (from 20 mg/kg to 10 mg/kg). The potentiation of the antiparkinsonian effect of levodopa in this combination cannot be explained by the additional antiparkinsonian effect of phenylephrine as the phenylephrine in dose 0.3 mg/kg has very weak antiparkinsonian effect.

The results of these studies indicate that “double” compositions containing levodopa and phenylephrine exert anti-parkinsonian effect which is vastly superior to the anti-parkinsonian effect of levodopa in the conventional dose alone, without developing hyperkinetic side effect and significantly reducing the dosage of conventional levodopa and phenylephrine in the composition. The above-mentioned compositions cause a potentiation of the anti-parkinsonian action of levodopa in a safe manner, since they eliminate the side effects of the use of each of the components of the composition.

### Example 7. Potentiation of Analgesic Effect and Reduction of Side Effects of Morphine in Tail-Flick and Open Field Tests (Intramuscular Administration)

### Methods.

### Tail-Flick Test.

Analgesic effect of drugs was estimated from prolongation of the tail-flick latency in male Wistar rats (Woolf C. J. et al., 1977, Eur. J. Pharmacol., Vol. 45(3), pages 311-314; Serdyuk S.E. and Gmiro V.E., 2007, Bull. Exp. Biol. Med., Vol. 143(3), pages 350-352). In the tail-flick test, pain is stimulated by rat tail immersion in hot water at 55±0.1° C. The latent period of withdrawal of the tail (time to get rid of the pain stimulus) is determined every 3 minutes. For evaluation of pain sensitivity, the rats that have short latency (3-5 seconds) during the last 15 minutes before administration of substances were used. Morphine, epinephrine, and morphine combination with epinephrine were administered intramuscular (IM) in a volume of 0.1-0.3 ml in increasing doses until a maximal analgesia (measured as maximal latency of tail withdrawal in seconds) during the last three measurements of pain sensitivity. Control animals were injected I.M. 0.2 ml of distilled water. Analgesic effect is assessed by an increase of latency of tail withdrawal compared to control.

### Open Field Test.

To evaluate the hypokinetic (sedative) effects of drugs, “open field” (OF) test was applied. In the OF test, the locomotive activity of rats is determined. Animals were placed in the center of square of the illuminated field (1 meter×1 meter) for 3 minutes and the mobility time was recorded in seconds (horizontal activity). The registration of horizontal locomotive activity was performed 40 minutes after administration of the test drug(s). To quantify the locomotive activity for each dose of test drug(s), an average horizontal activity was calculated. Hypokinetic (sedative) action of morphine, epinephrine, and combination of morphine with epinephrine was assessed by reduction (hypokinesia) of average horizontal activity in the OF test in % compared with measurements of rats in the control group as well as the number of rats with a significant decrease in the horizontal activity (50% and more as compared to the control).

TABLE 8

| | | | Locomotive horizontal | | | |
|---|---|---|---|---|---|---|
| Morphine | Epinephrine | Latent period | activity in open field | Sedation | Morphine | Epinephrine |
| (mg/kg) | (mg/kg) | (seconds) | (sec) | (% of group) | (mg, Human Dose)** | (mg, Human Dose)** |
| | | 4.5* | 15.6* | 0% | | |
| 1 | | 6.9 | 14.8 | 0% | 9.67 | |
| 2.5 | | 11.7 | 12.5 | 20% | 24.19 | |
| 5 | | 19.8 | 6.1 | 90% | 48.38 | |
| | 0.01 | 5 | 15.4 | 0% | | 0.096 |
| | 0.03 | 9.2 | 15.9 | 0% | | 0.29 |
| | 0.1 | 18.2 | 17.6 | 0% | | 0.96 |
| 0.2 | 0.01 | 8.7 | 15.2 | 0% | 1.93 | 0.096 |
| 0.4 | 0.01 | 18.9 | 14.9 | 0% | 3.87 | 0.096 |
| 5 | 0.01 | 21.5 | 9.6 | 50% | 48.38 | 0.096 |
| 2.5 | 0.03 | 26.7 | 15.7 | 0% | 24.19 | 0.29 |
| 5 | 0.03 | 32.7 | 13.6 | 0% | 48.38 | 0.29 |

*N.C.—Negative control (distilled water).
**Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).

### Dual-Therapy.

Combined administration of morphine at a very low dose of 0.2 mg/kg and epinephrine in threshold dose of 0.01 mg/kg (each ineffective alone) insignificantly reduced the pain sensitivity (1.9 fold compared with control) as it increased the latency of the tail flick from 4.5 to 8.7 seconds. Combined administration of morphine at a higher dose of 0.4 mg/kg with epinephrine in threshold dose of 0.01 mg/kg induced a maximal analgesic effect (4.2 fold, increasing the latency of the tail flick from 4.5 to 18.9 seconds), such as morphine alone in high dose of 5 mg/kg. Such a combination did not reduce horizontal activity in the rat. Hence, this combination the effective dose of morphine by 12.5 times, causing maximal analgesic effect without developing side hypokinetic (sedative) effect.

Combined administration of morphine at a high dose of 5 mg/kg with epinephrine in threshold dose of 0.01 mg/kg did not significantly increase the maximal analgesic effect of morphine alone (increases the latency of the tail flick from 19.8 to 21.5 seconds), but eliminated hypokinesia in 40% of the rats. Hence, epinephrine in threshold dose of 0.01 mg/kg did not increase the analgesic effect of morphine at a high dose, but partly eliminates the side hypokinetic effect of morphine.

Combined administration of morphine at both a medium dose of 2.5 mg/kg and a higher dose of 5 mg/kg with epinephrine in conventional low dose 0.03 mg/kg (having light analgesic effect alone) increased the maximal analgesic effect of morphine 1.4 and 1.7 times, respectively (increased latency of tail withdrawal from 19.8 sec to 26.7 and 32.7 seconds, respectively), as well as eliminated hypokinesia in 100% of the rats. Thus epinephrine in conventional low dose 0.03 mg/kg significantly increased the maximum analgesic effect of morphine in conventional doses of 2.5 and 5 mg/kg without developing side hypokinetic (sedative) effect.

The potentiated synergism of intramuscularly administered combination of morphine with low conventional dose of epinephrine (0.03 mg/kg) is highly surprising, comprising 1.4-1.7 fold increase in the maximal analgesic effect and a 2 fold decrease in the conventional dose of morphine (from 5 mg/kg to 2.5 mg/kg) achieving potentiated analgesic effect. The potentiation of the analgesic effect of morphine in this combination cannot be explained by the additional analgesic effect of epinephrine as the epinephrine in a dose of 0.03 mg/kg has very weak analgesic effect.

### Example 8. Potentiation of Analgesic Effect and Reduction of Side Effects of Morphine in Tail-Flick and Open Field Tests (Oral Administration)

### Methods.

Tail-Flick and Open Field Tests were Conducted as Detailed Above.

Morphine, phenylephrine and a combination of morphine with epinephrine were administered orally in a volume of 1.0 ml through a rigid metal probe, in increasing doses until a maximal analgesia (measured as maximal latency of tail withdrawal in seconds) during the last three measurements of pain sensitivity, 45 minutes before the administration of haloperidol was achieved. Control animals received orally 1 ml of distilled water.

TABLE 9

| | | | Horizontal | | | |
|---|---|---|---|---|---|---|
| Morphine | Phenylephrine | Latent period | locomotive activity | Sedation | Morphine | Phenylephrine |
| (mg/kg) | (mg/kg) | (seconds) | (seconds) | (% of group) | (mg, Human Dose)** | (mg, Human Dose)** |
| | | 4.6* | 16.1* | 0% | | |
| 0.3 | | 6.5 | 15.5 | 0% | 2.88 | |
| 1.0 | | 10.8 | 13.2 | 10% | 9.6 | |
| 3.0 | | 19 | 7.8 | 70% | 28.8 | |
| | 0.02 | 5.5 | 15.9 | 0% | | 0.19 |
| | 0.1 | 9.4 | 16.3 | 0% | | 0.96 |
| | 0.3 | 18.4 | 17.9 | 0% | | 2.88 |
| 0.1 | 0.02 | 8.5 | 15 | 0% | 0.96 | 0.19 |
| 0.2 | 0.02 | 19.4 | 14.7 | 0% | 1.92 | 0.19 |
| 3.0 | 0.02 | 22 | 9.4 | 50% | 28.8 | 0.19 |
| 1.0 | 0.1 | 29.4 | 16.2 | 0% | 9.6 | 0.96 |
| 3.0 | 0.1 | 35.5 | 13.9 | 0% | 28.8 | 0.96 |

*N.C.—Negative control (distilled water).
**Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).

### Dual-Therapy.

Morphine in a very low dose of 0.1 mg/kg combined with phenylephrine in threshold dose of 0.02 mg/kg (each ineffective independently) insignificantly reduced pain sensitivity by only a 1.8 fold compared with control, as it increases the latency of the tail flick from 4.6 to 8.5 seconds. Morphine at a higher dose of 0.2 mg/kg combined with phenylephrine at a threshold dose of 0.02 mg/kg reached the maximal analgesic effect (4.2 fold increase compared to control, the latency of the tail flick increased from 4.6 to 19.4 seconds), the same as the high dose of morphine in the 3 mg/kg alone. As presented in Table 3, such a combination did not reduce the horizontal activity in rats. Hence, the threshold dose of phenylephrine of 0.02 mg/kg reduced the effective dose of morphine by 15 times, causing maximal analgesic effect without developing side hypokinetic (sedative) effect.

Morphine at a high dose of 3 mg/kg combined with phenylephrine at a threshold dose of 0.02 mg/kg insignificantly further increased the maximal analgesic effect of morphine by only a 1.2 fold (increases the latency of the tail flick from 19 to 22 seconds), but eliminated hypokinesia in 20% of rats (reduced from 70% to 50%). Hence, phenylephrine in the threshold dose of 0.02 mg/kg practically did not increase the analgesic effect of morphine at a high dose, but partially eliminates the sedative side effect of morphine. Morphine in conventional low dose of 1 mg/kg as well as in high conventional dose of 3 mg/kg combined with phenylephrine in the conventional low dose of 0.1 mg/kg (having a light analgesic effect alone) increased the maximal analgesic effect of morphine 1.6 and 1.9 times, respectively (increase latency of the tail withdrawal from 19 seconds to 29.4 and 35.5 seconds, respectively), and also eliminated hypokinesia in all rats. Thus, the combined oral administration of morphine in a conventional lowest dose of 1 mg/kg and in a high conventional dose of 3 mg/kg with phenylephrine in the low conventional dose of 0.1 mg/kg increased the maximal analgesic effect that is ordinarily achieved upon administration of 3 mg/kg morphine by 60-90%, without development of side effect (sedation).

The potentiated synergism of orally administered combination of morphine with low conventional dose of phenylephrine (0.1 mg/kg) is highly surprising, comprising 1.5-2 fold increase in the maximum analgesic effect and a 3 fold decrease in the conventional dose of morphine (from 3 mg/kg to 1 mg/kg). The potentiation of the analgesic effect of morphine in this combination cannot be explained by the additional analgesic effect of phenylephrine, as the phenylephrine in a dose 0.1 mg/kg has very weak analgesic effect.

### Example 9. Potentiation of the Anti-Parkinsonian and Neuro-Protective Effects, and Elimination of Side Effects, of Levodopa in Rotenone-Induced Parkinson Disease (PD) and Progressive Supranuclear Palsy (PSP) in Rats (Oral Administration)

### Administration of Rotenone.

Rotenone was dissolved in mixture of DMSO and Miglyol 812N at a ratio of 2:98 at concentration of 2.3 mg/ml. Preparation: 230 mg sample of rotenone in vials was dissolved in 2 ml of DMSO. The solution was poured into a jar with 100 ml. Vials are rinsed twice with 5 ml of Miglyol, washings attached to the main solution and stirred. To this solution was added 88 ml of Miglyol and mixed again. The resulting solution was divided into 3 doses. The solution was stored at −10° C. Each animal was administered 0.2 ml of a solution of rotenone: a dose of 0.46 mg per animal weighing 200±20 g, i.e. 2.3 mg/kg. The solution of rotenone was injected IP one time a day for 19 days.

Anti-parkinsonian effect of therapy in rats with rotenone-induced parkinsonism was estimated according to elimination of extrapyramidal disorders (catalepsy) and oligokinesia in the “open field” test. The neuro-protective effect of therapy was evaluated by the elimination of ataxia and mortality in rats with rotenone-induced PSP. Hyperkinetic side action (hyper-locomotion) was evaluated by increased horizontal and vertical activity in the open field at the 10th and 18th day of the experiment, calculated in % compared to the first day of the experiment.

Phenylephrine 0.3 mg/kg, levodopa (10 and 20 mg/kg), a combination of levodopa (10 mg/kg)+phenylephrine (0.3 mg/kg), a combination of levodopa (20 mg/kg)+phenylephrine (0.3 mg/kg) and DW (1.0 ml, control) were orally administered using rigid metal probe in volumes of 1 ml daily for 19 days, 45 minutes before administration of rotenone. The number of animals in the control and experimental groups ranged from 7 to 8.

Levodopa was administered in combination with benserazide (peripheral dopa-decarboxylase inhibitor) at a ratio of 4:1 to reduce the side effects of levodopa, associated with the stimulation of peripheral dopamine receptors (Alam M. et al., Behav. Brain Res., 2004, Vol. 153 (2), pages 439-446). Levodopa with benserazide were dissolved in 1 ml of DW and administered orally in the volume of 1 ml. Combination of levodopa with phenylephrine, of levodopa and of benserazide solution (0.5 ml) was mixed with 0.5 ml of phenylephrine solution. The mixture was administered orally in a volume of 1 ml. Anti-parkinsonian effect in the experimental groups of rats with rotenone has been evaluated by eliminating catalepsy and oligokinesia.

Catalepsy in rats is determined by the time of immobilization of the animal, placed on a large grid predisposed at an angle of 45°. Maximum catalepsy in rats is recorded in the case of their complete immobilization on grid at 120 seconds for observation. The value of catalepsy is evaluated in points: 3 points—immobilization duration from 80 seconds to 120, 2 points—immobilization duration from 40 to 70, 1 point—immobilization duration from 20 to 35, 0 points—the immobilization of less than 20 seconds. Catalepsy was followed in rats daily, 30 minutes before administration of rotenone and 180 minutes after administration of rotenone. For each experimental group, on a daily basis, throughout the period of observation number of rats with severe catalepsy (2-3 points) was determined in % of the total number of rats in group. To assess the anti-cataleptic effects of therapy, on 12th and 19th day of experiment, reduction in the number of rats with severe catalepsy (2-3 points) in % was determined compared to control treated with distilled water.

Locomotive activity of rats was tested in the “open field” test. Animals were placed in the center of square of the illuminated field (1 meter×1 meter) and during 3 minutes the distance of movement (horizontal activity) and the number of rearing (vertical activity) was recorded. Registration of horizontal and vertical locomotive activity was carried out in first day for rats with high selection locomotive activity (total walking time with not less than 12 seconds and the number of vertical uprights at least 4), 60 minutes before the first administration of rotenone. To identify the rotenone-induced oligokynesia in each experimental group the locomotive activity in the open field test was examined again 2 hours after the administration of rotenone on days 12 and 19 of the experiment.

Quantification of oligokynesia in points: 0 points—the highest horizontal activity (walking total time more than 12 seconds) and high vertical activity (number of vertical posts) more than 3; 1 point—reducing the horizontal activity (total walking time 7-11 seconds) and a decrease in vertical activity (the number of vertical columns 1-3); 2 points—a significant reduction in horizontal activity (total walking time 2-6 seconds) and the absence of vertical activity (the number of vertical columns 0-1); 3 points—the absence of horizontal activity (total walking time 0-1 seconds) and the absence of vertical activity (number of vertical columns 0). In each experimental group 120 minutes after administration of rotenone on days 12 and 19 of experiment the number of rats with severe oligokinesia (2-3 points) in % of the total number of rats in group was determined.

The neuro-protective effect in the experimental groups of rats with rotenone-induced PSP was evaluated due the elimination of ataxia and mortality caused by neurotoxic action of rotenone. Ataxia is typical incoordination neurotoxic effect of rotenone manifested in uncoordinated movements and hyper-locomotion, replaced by hypokinesia and stereotypes, and in severe cases a complete violation of antigravity reflexes, falling on its side and akinesia. Assessment of the severity of ataxia in scores was performed using the following scale: (+) uncoordinated movement, hyperkinesia; (++) significant incoordination, hypokinesia, stereotypes, falling on its side; (+++) complete violation of antigravity reflexes, akinesia. Ataxia registration was performed twice daily, 30 minutes before administration of rotenone and 180 minutes after administration of rotenone. For each experimental group on a daily basis, throughout the whole period of observation number of rats with severe ataxia (++−+++) and mortality in % of the total number of rats in group was determined. The neuro-protective effect of therapy (effects on rate of ataxia and lethality) in rats with PSP was determined at days 12 and 19 of the experiment as reducing the number of rats with severe ataxia and mortality in % compared to control. Side hyperkinetic effects were evaluated in the “open field” test. In this test the locomotive activity in rats is determined. Animals were placed in the center of square of the illuminated field (1 meter×1 meter) for 3 min total time recorded in seconds distance (horizontal activity) and the number of rearing (vertical activity). Registration of horizontal and vertical locomotive activity in OF assay was performed on the 10th and 18th day of the experiment, within 40 minutes after administration of the substance and 5 minutes before administration of rotenone. Side hyperkinetic effects was evaluated by the number of rats with a significant (at least 50% compared with the first day of the experiment) increase in horizontal and vertical activity (hyperkinesia) as a % of the total number of rats in group.

TABLE 10
Potentiation of the anti-parkinsonian and neuro-protective effects, and elimination
of side effects of levodopa¹ in rotenone-induced PD and PSP in rats.

| | | Severe | | Severe | | Severe ataxia | | | | | |
|---|---|---|---|---|---|---|---|---|---|---|---|
| Doses for rat | | catalepsy* | | oligokinesia* | | (% of group) | | Hyperkinesia**** | | Doses for human | |
| (mg/kg) | | (% of group) | | (% of group) | | [lethality] | | (% of group) | | (mg) | |
| Levodopa | Phenylephrine | Day 12 | Day 19 | Day 12 | Day 19 | Day 12 | Day 19 | Day 10 | Day 18 | Levodopa² | Phenylephrine² |
| | | 48³ | 58³ | 100³ | 100³ | 14[0]³ | 42[14]³ | 0³ | 0³ | | |
| | 0.3 | 36 | 42 | 90 | 100 | 14[0] | 28[14] | 0 | 0 | | 2.9 |
| 10 | | 36 | 42 | 90 | 100 | 14[0] | 42[14] | 0 | 0 | 96.7 | |
| 20 | | 12 | 24 | 50 | 60 | 0[0] | 24[0] | 90 | 75 | 193.5 | |
| 10 | 0.3 | 0 | 12 | 25 | 25 | 0 | 0 | 0 | 0 | 96.7 | 2.9 |
| 20 | 0.3 | 0 | 0 | 14 | 14 | 0 | 0 | 14 | 0 | 193.5 | 2.9 |

¹Oral administration 45 minutes before the administration of rotenone 2.3 mg/kg in duration 19 days.
²Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).
³N.C.—Negative control (distilled water).
*The immobilization time of a rat on an inclined grid above 40 seconds.
**Oligokinesia ++-+++ in OF test.
***Ataxia ++-+++.
****Increased locomotive activity in OF test more than 50% compared to the first day of experiment.

### Dual-Therapy.

A combination of levodopa (10 mg/kg) with phenylephrine (0.3 mg/kg) upon chronic oral administration on days 12 and 19 of experiment had reduced the number of rats with severe oligokinesia by 75% compared to control (from 100% to 25%) indicating a very significant reduction of oligokinesia in rats with rotenone-induced PD. This combination on days 12 and 19 of the experiment reduced the number of rats with severe catalepsy with maximum efficiency to 48% and 46% respectively compared to control (from 48% to 0% and from 58% to 12%). A combination of levodopa 10 mg/kg and phenylephrine 0.3 mg/kg completely eliminated ataxia and mortality at days 12 and 19 of experiment. A combination of levodopa in a dose of 10 mg/kg and phenylephrine in a dose of 0.3 mg/kg did not cause hyperkinesia in rats in the OF test on the 10th and 18th day of experiment. Thus, the combination of levodopa in a conventional low dose (10 mg/kg) and phenylephrine in the low convention dose of 0.3 mg/kg eliminated catalepsy and oligokynesia in rats with rotenone-induced PD and completely eliminates ataxia and paralysis in rats with rotenone-induced PSP. The advantage of the aforementioned combination is a complete absence of hyperkinetic side effect in rats.

Chronic oral administration of combination of levodopa in a dose of 20 mg/kg with phenylephrine in a dose of 0.3 mg/kg reduced the number of rats with severe oligokinesia by 86% compared to control (from 100% to 14%). This indicates that this combination eliminated oligokinesia in rats with rotenone-induced PD. This combination completely eliminated heavy catalepsy in rats with rotenone-induced PD (decrease from 48% to 0% and from 58% to 0%). A combination of levodopa in a dose of 20 mg/kg and phenylephrine in a dose of 0.3 mg/kg completely eliminated ataxia and mortality on days 12 and 19 of experiment. A combination of levodopa in a dose of 20 mg/kg and phenylephrine in a dose of 0.3 mg/kg caused an insignificant increase of locomotive activity only in 14% and 0% of the rats on the 10th and 18th day of the experiment, that is 76% less compared with the use of levodopa 20 mg/kg alone. Therefore, chronic orally administered combination of levodopa in a high conventional dose of 20 mg/kg and phenylephrine in a conventional low dose 0.3 mg/kg to rats efficiently eliminated the extrapyramidal and motor disorders in rats with rotenone-induced PD and completely eliminated ataxia and paralysis in rats with rotenone-induced PSP, and virtually eliminates the hyperkinesia induced by a high dose of levodopa. Thus, oral administration of levodopa in the low conventional dose 10 mg/kg and in the high conventional dose of 20 mg/kg with phenylephrine in a low dose of 0.3 mg/kg to rats increases the anti-parkinsonian and neuro-protective effect that is achieved by levodopa in a dose of 20 mg/kg by 50-100% without developing side hyperkinetic effects. The anti-parkinsonian and neuro-protective activity of the combination of levodopa in a conventional lowest dose of 10 mg/kg with phenylephrine in a conventional lowest dose of 0.3 mg/kg is highly surprising, as it far exceeds the anti-parkinsonian and neuro-protective activity of each one of the components of the combination alone, as well as it surpasses (by 1.5-2 times) the anti-parkinsonian and neuro-protective activity of levodopa in a double dose of 20 mg/kg. Therefore, phenylephrine that is not effective alone in a dose of 0.3 mg/kg synergistically potentiated the anti-parkinsonian and neuro-protective effects of levodopa in a low dose of 10 mg/kg that had low efficacy while being used alone, to the maximal level, which cannot be explained in view of the independent use of levodopa in the high dose of 20 mg/kg alone. However, phenylephrine did not enhance the hyperkinetic side effect of levodopa at dose of 10 mg/kg.

It is important to notice the relatively small difference in neuro-protective and anti-parkinsonian activity of the combination of phenylephrine with levodopa at a dose of 20 mg/kg and 10 mg/kg. This implies that much less phenylephrine enhanced the therapeutic effects of levodopa in the high dose of 20 mg/kg, but effectively eliminated the hyperkinetic side effects of levodopa in high dose of 20 mg/kg. According to the results of the experiments, the combination of levodopa with phenylephrine in conventional low doses can be offered for the safe treatment of severe Parkinson's disease and supranuclear progressive palsy, resistant to the action of levodopa, as well as for the treatment of Parkinson's disease and PSP in patients who cannot tolerate levodopa.

### Example 10. Potentiation of Analgesic and Anti-Hyperalgesic Effects, and Reduction of Side Effects of Memantine, Morphine, Amitriptyline and Dipyrone in Tail-Flick, Paw Withdrawal and Open Field Tests

### Methods.

### Tail-Flick Test (Acute Pain).

Acute analgesic action of drugs was estimated from prolongation of the tail-flick latency in male Wistar rats (Woolf C. J. et al., 1977, Eur. J. Pharmacol., Vol. 45(3), pages 311-314; Serdyuk S.E. and Gmiro V.E., 2007, Bull. Exp. Biol. Med., Vol. 143(3), pages 350-352). In the tail-flick test, pain is stimulated by rat tail immersion in hot water at 55±0.1° C. The latent period of withdrawal of the tail (time to get rid of the pain stimulus) is determined every 3 minutes. For evaluation of pain sensitivity the rats that have short latency (3-5 seconds) during the last 15 minutes before administration of substances were used. Morphine, memantine, amitriptyline, dipyrone, phenylephrine, epinephrine, as well as double combinations of thereof were administered intramuscular (I.M.) or orally in increasing doses until a maximal analgesia (measured as maximal latency of tail withdrawal in seconds) during the last three measurements of pain sensitivity. Control animals were injected I.M. 0.2 ml or orally 1.0 ml of distilled water. Analgesic effect is assessed by an increase of latency of tail withdrawal (s) compared to control (dist. Water).

### Paw Withdrawal Test (Acute Inflammatory Hyperalgesia).

Inflammatory hyperalgesia of a paw was caused by placing it into hot water (56° C.) for 20-25 seconds under the conditions of ether anesthesia. Hyperalgesia was developed 30 min after the burn (latency of paw withdrawal on its being placed into water at a temperature 47° C. was reduced from 15-20 seconds to 3-4 seconds). Acute anti-hyperalgesic action of drugs was estimated from prolongation of the paw withdrawal latency in male Wistar rats with inflammatory hyperalgesia (Coderre T. J., Melzack R. Brain Res. (1987) 404(1-2):95-106).

The latent period of paw withdrawal (time to get rid of the pain stimulus) is determined every 5 minutes. For estimation of hyperalgesia the rats that have short latency (3-5 seconds) during the last 20 min before administration of substances were used. Morphine, memantine, amitriptyline, dipyrone, phenylephrine, epinephrine, as well as double combinations thereof were administered intramuscular (IM) or orally in increasing doses until a maximal latency of paw withdrawal in seconds during the last three measurements of pain sensitivity. Control animals were injected I.M. 0.2 ml or orally 1.0 ml of distilled water. Anti-hyperalgesic effect is assessed by an increase of latency of paw withdrawal (s) compared to control (dist. Water).

### Open Field Test.

To evaluate the hypokinetic (sedative) or hyperkinetic action effects of tested drugs an “open field” (OF) test was applied. In the OF test, the locomotive activity of rats is determined. Animals were placed in the center of square of the illuminated field (1 meter×1 meter) for 3 minutes and the mobility time was recorded in seconds (horizontal activity). The registration of horizontal locomotive activity was performed 40 minutes after administration of the test drug(s). To quantify the locomotive activity for each dose of test drug(s) an average horizontal activity was calculated. Hypokinetic (sedative) or hyperkinetic action of morphine, memantine, epinephrine, phenylephrine, amitriptyline and dipyrone and their double combinations were assessed by reduction (hypokinesia) or increase (hyperkinesia) of average horizontal activity in the OF test in % compared with measurements of rats in the control group as well as the number of rats with a significant decrease or increase the horizontal activity (50% and more as compared to the control).

TABLE 11

| | | | Tail flick | Pow withdrawal | | |
|---|---|---|---|---|---|---|
| Morphine | Memantine | Epinephrine | latency | latency | Hyperkinesia | Hyperalgesia |
| (Rat mg/kg) | (Rat mg/kg) | (Rat mg/kg) | (seconds) | (seconds) | (% rats) | (% rats) |
| | | | 4.5* | 4.3* | 0 | 0 |
| 0.01 | | | 1.2 | 1.3 | 0 | 90 |
| 0.02 | | | 0.9 | 1.1 | 0 | 100 |
| 0.2 | | | 4.9 | 5.2 | 0 | 0 |
| 1 | | | 6.9 | 7.2 | | 0 |
| 2.5 | | | 11.7 | 11.3 | 20** | 0 |
| 5 | | | 19.8 | 19.5 | 90** | 0 |
| | | 0.01 | 5 | 4.8 | 0 | 0 |
| | | 0.03 | 9.2 | 7.6 | 0 | 0 |
| | | 0.1 | 18.2 | 12.3 | 0 | 0 |
| | 1 | | 6.0 | 6.3 | 0 | 0 |
| | 2.5 | | 8.1 | 7.5 | 10*** | 0 |
| | 5 | | 12.7 | 12.2 | 20*** | 0 |
| | 15 | | 19.1 | 18.8 | 90*** | 0 |
| | 1 | 0.01 | 6.5 | 5.9 | 0 | 0 |
| | 2.5 | 0.01 | 19.7 | 19.3 | 0 | 0 |
| | 5 | 0.01 | 27.5 | 27.2 | 0 | 0 |
| 0.02 | | 0.01 | 1.5 | 1.4 | 0 | 80 |
| 0.02 | | 0.02 | 6.5 | 6.2 | 0 | 0 |
| 0.2 | | 0.01 | 8.7 | 8.2 | 0 | 0 |
| 0.4 | | 0.01 | 18.9 | 18.4 | 0 | 0 |
| 5 | | 0.01 | 28.5 | 28.2 | 0 | 0 |

*N.C.—Negative control (distilled water).
**Sedation in open field test.
***Hyperkinesia in open field test.

### Dual-Therapy.

Combined administration of morphine at a very low dose of 0.02 mg/kg and epinephrine in threshold dose of 0.01 mg/kg not eliminated opioid hyperalgesia causing morphine in dose 0.02 mg/kg in tail and paw withdrawal test Combined administration of morphine at a very low dose of 0.02 mg/kg and epinephrine in higher threshold dose of 0.02 mg/kg eliminated opioid hyperalgesia causing morphine in dose 0.02 mg/kg as it increased the latency of the tail and paw withdrawal from 0.9 and 1.1 s to 6.5 and 6.2 seconds

Combined administration of morphine at a low dose of 0.2 mg/kg and epinephrine in threshold dose of 0.01 mg/kg (each ineffective alone) insignificantly reduced the pain sensitivity and hyperalgesia (1.9 and 1.8 fold compared with control) as it increased the latency of the tail and paw withdrawal from 4.5 and 4.3 seconds to 8.7 and 8.2 seconds. Combined administration of morphine at a higher dose of 0.4 mg/kg with epinephrine in threshold dose of 0.01 mg/kg induced a maximal analgesic and anti-hyperalgesic effect reducing the pain sensitivity and hyperalgesia by 4.2 and 4.3 folds (increase the latency of the tail paw withdrawal respectively from 4.5 and 4.3 seconds to 18.9 and 18.4 seconds), such as morphine alone in high dose of 5 mg/kg. Such a combination did not reduce horizontal activity in the rat. Hence, this combination decreased the effective dose of morphine by 12.5 times, causing maximal analgesic effect without developing side hypokinetic (sedative) effect.

Combined administration of morphine at a high dose of 5 mg/kg with epinephrine in threshold dose of 0.01 mg/kg increased the maximal analgesic and anti-hyperalgesic effect of morphine by 1.4 and 1.5 times (increased latency of tail and paw withdrawal from 19.8 and 19.5 seconds respectively to 28.5 and 28.2 seconds), as well as eliminated hypokinesia in 100% of the rats.

Thus epinephrine in threshold dose 0.01 mg/kg significantly increased the maximum analgesic effect of morphine in high dose 5 mg/kg without developing side hypokinetic (sedative) effect.

The potentiated synergism of intramuscularly administered combination of morphine with threshold dose of epinephrine (0.01 mg/kg) is highly surprising, because decrease in 12.5 times effective dose of morphine (from 5 mg/kg to 0.4 mg/kg) and 1.4-1.5 fold increase of the maximal analgesic and anti-hyperalgesic effect of high dose of morphine. Potentiation cannot be explained by the additional analgesic effect of epinephrine as the epinephrine in a dose of 0.01 mg/kg not has analgesic and anti-hyperalgesic effect.

Combined administration of memantine at a low dose of 1 mg/kg and epinephrine in threshold dose of 0.01 mg/kg (each ineffective alone) practically did not reduce the pain sensitivity and hyperalgesia compared with the control (the latency of the tail and paw withdrawal changed from 4.5 and 4.3 seconds to 6.5 and 5.9 seconds). Combined administration of memantine at a higher dose of 2.5 mg/kg with epinephrine in threshold dose of 0.01 mg/kg induced a maximal analgesic and anti-hyperalgesic effect (4.4 and 4.5 folds, increasing the latency of the tail and paw withdrawal respectively from 4.5 and 4.3 seconds to 19.7 and 19.3 seconds, such as memantine alone in high dose of 15 mg/kg. Such a combination did not increase horizontal activity in the rat. Hence, this combination decreased the effective dose of memantine by 6 times, causing maximal analgesic effect without developing side hyperkinetic effect.

Combined administration of memantine at a middle dose of 5 mg/kg with epinephrine in threshold dose of 0.01 mg/kg increased the maximal analgesic and anti-hyperalgesic effect of memantine in maximal dose 15 mg/kg by 1.4 and 1.4 times (increased latency of tail and paw withdrawal from 19.1 and 18.8 seconds respectively to 27.5 and 27.2 s), as well as eliminated hyperkinesia in 100% of the rats.

Thus epinephrine in threshold dose 0.01 mg/kg significantly increased the maximum analgesic effect and decrease effective dose of memantine without developing side hyperkinetic effect.

The potentiated synergism of intramuscularly administered combination of memantine with threshold 0.1 mg/kg of epinephrine is highly surprising, comprising a 1.4 fold increase in the maximal analgesic and anti-hyperalgesic effect and a 3 fold decrease in the convention's dose of memantine (from 15 mg/kg to 5 mg/kg). The potentiation of the analgesic and anti-hyperalgesic effect of memantine in this combination cannot be explained by the additional analgesic effect of epinephrine as the epinephrine in dose 0.1 mg/kg not has analgesic and anti-hyperalgesic effect.

The results of these studies indicate that “double” compositions containing epinephrine with morphine and memantine exert analgesic and anti-hyperalgesic effect which is vastly superior to the analgesic and anti-hyperalgesic effect of morphine and memantine in the conventional dose alone, without developing side sedative and hyperkinetic action and significantly reducing of the conventional doses of morphine, memantine and epinephrine in the composition. The above-mentioned compositions cause a potentiation of the analgesic and anti-hyperalgesic action of morphine and memantine in a safe manner, since they eliminate the side effects of the use of each of the components of the composition.

TABLE 12

| | | | | | Tail flick | Pow withdrawal | | |
|---|---|---|---|---|---|---|---|---|
| Morphine | Memantine | Phenylephrine | Amitriptyline | Dipyrone | latency | latency | Hyperkinesia | Hyperalgesia |
| (Rat mg/kg) | (Rat mg/kg) | (Rat mg/kg) | (Rat mg/kg) | (Rat mg/kg) | (seconds) | (seconds) | (% rats) | (% rats) |
| | | | | | 4.8* | 4.5* | | |
| 0.01 | | | | | 1.3 | 1.2 | | 90 |
| 0.02 | | | | | 1.1 | 1.0 | 0 | 100 |
| 1 | | | | | 8.5 | 8.3 | 10** | 0 |
| 5 | | | | | 20.5 | 21.3 | 80** | 0 |
| | | 0.02 | | | 4.4 | 4.6 | 0 | 0 |
| | | 0.04 | | | 6.6 | 6.8 | 0 | 0 |
| | | 0.1 | | | 18.7 | 11.3 | 0 | 0 |
| | 1 | | | | 4.3 | 4.0 | 0 | 0 |
| | 5 | | | | 8.3 | 9.0 | 20*** | 0 |
| | 20 | | | | 20.3 | 21.0 | 90*** | 0 |
| | | | 0.1 | | 4.5 | 4.7 | 0 | 0 |
| | | | 0.5 | | 5.5 | 5.3 | 0 | 0 |
| | | | 2.0 | | 7.5 | 7.3 | 10** | 0 |
| | | | 10.0 | | 20.2 | 19.8 | 90** | 0 |
| | | | | 0.2 | 4.3 | 4.6 | 0 | 0 |
| | | | | 1.0 | 5.3 | 5.5 | 0 | 0 |
| | | | | 4.0 | 7.7 | 7.4 | 0 | 0 |
| | | | | 20.0 | 20.6 | 20.1 | 0 | 0 |
| | 1 | 0.02 | | | 5.2 | 5.4 | 0 | 0 |
| | 2 | 0.02 | | | 20.2 | 19.4 | 0 | 0 |
| | 5 | 0.02 | | | 28.9 | 28.4 | 0 | 0 |
| 0.02 | | 0.02 | | | 1.5 | 1.4 | 0 | 80 |
| 0.02 | | 0.04 | | | 7.5 | 7.2 | 0 | 0 |
| 0.1 | | 0.02 | | | 8.5 | 7.9 | 0 | 0 |
| 0.2 | | 0.02 | | | 19.4 | 19.1 | 0 | 0 |
| 5.0 | | 0.02 | | | 30.5 | 29.8 | 0 | 0 |
| | | 0.02 | 0.1 | | 6.5 | 6.7 | 0 | 0 |
| | | 0.02 | 0.5 | | 19.3 | 18.9 | 0 | 0 |
| | | 0.02 | 10.0 | | 29.0 | 28.8 | 0 | 0 |
| | | 0.02 | | 0.2 | 6.9 | 6.6 | 0 | 0 |
| | | 0.02 | | 1.0 | 19.3 | 19.0 | 0 | 0 |
| | | 0.02 | | 20.0 | 29.4 | 29.2 | 0 | 0 |

*N.C.—Negative control (distilled water).
**Sedation in open field test.
***Hyperkinesia in open field test.

### Dual-Therapy.

Combined administration of morphine at a very low dose of 0.02 mg/kg and phenylephrine in threshold dose of 0.02 mg/kg not eliminated opioid hyperalgesia causing morphine in dose 0.02 mg/kg in tail and paw withdrawal test. Combined administration of morphine at a very low dose of 0.02 mg/kg and phenylephrine in higher threshold dose of 0.04 mg/kg eliminated opioid hyperalgesia causing morphine in dose 0.02 mg/kg as it increased the latency of the tail and paw withdrawal from 1.1 and 1.0 seconds respectively to 7.5 and 7.2 seconds.

Combined administration of morphine at a low dose of 0.1 mg/kg and phenylephrine in threshold dose of 0.02 mg/kg (each ineffective alone) insignificantly reduced the pain sensitivity and hyperalgesia (by 1.8 and 1.8 fold compared with control) as it increased the latency of the tail and paw withdrawal from 4.8 and 4.5 seconds to 8.5 and 7.9 seconds. Combined administration of morphine at a higher dose of 0.2 mg/kg with phenylephrine in threshold dose of 0.02 mg/kg induced a maximal analgesic and anti-hyperalgesic effect reducing pain sensitivity and hyperalgesia by 4.0 and 4.2 folds, (increase the latency of the tail paw withdrawal respectively from 4.8 and 4.5 seconds respectively to 19.4 and 19.1 seconds), such as morphine alone in high dose of 5 mg/kg. Such a combination did not reduce horizontal activity in the rat. Hence, this combination decreased the effective dose of morphine by 25 times, causing maximal analgesic effect without developing side hypokinetic (sedative) effect.

Combined administration of morphine at a high dose of 5 mg/kg with phenylephrine in threshold dose of 0.02 mg/kg increased the maximal analgesic and anti-hyperalgesic effect of morphine by 1.5 and 1.4 times as it increased latency of tail and paw withdrawal from 20.5 and 21.3 seconds respectively to 30.5 and 29.8 seconds, as well as eliminated hypokinesia in 100% of the rats.

Thus, phenylephrine in threshold dose 0.02 mg/kg significantly increased the maximum analgesic effect of morphine in high dose 5 mg/kg without developing side hypokinetic (sedative) effect.

The potentiated synergism of orally administered combination of morphine with threshold dose of phenylephrine (0.02 mg/kg) is highly surprising, because decrease in 25 times effective dose of morphine (from 5 mg/kg to 0.4 mg/kg) and 1.5-1.4 folds increase of the maximal analgesic and anti-hyperalgesic effect of high dose of morphine (5 mg/kg). Potentiation cannot be explained by the additional analgesic effect of phenylephrine as the phenylephrine in a dose of 0.02 mg/kg not has analgesic and anti-hyperalgesic effect.

Combined administration of memantine at a low dose of 1 mg/kg and phenylephrine in threshold dose of 0.02 mg/kg practically did not change the sensitivity to pain and hyperalgesia (the latency of the tail and paw withdrawal changed from 4.8 and 4.5 seconds to 5.2 and 5.4 seconds). Combined administration of memantine at a higher dose of 2 mg/kg with phenylephrine in threshold dose of 0.02 mg/kg induced a maximal analgesic and anti-hyperalgesic effect reducing pain sensitivity and hyperalgesia by 4.2 and 4.3 folds (increase the latency of the tail paw withdrawal respectively from 4.8 and 4.5 seconds to 20.2 and 19.4 seconds), such as memantine alone in high dose of 20 mg/kg. Such a combination did not increase horizontal activity in the rat. Hence, this combination decreased the effective dose of memantine by 10 times, causing maximal analgesic effect without developing side hyperkinetic effect.

Combined administration of memantine at a middle dose of 5 mg/kg with phenylephrine in threshold dose of 0.02 mg/kg increased the maximal analgesic and anti-hyperalgesic effect of memantine in high dose 20 mg/kg by 1.4 and 1.4 times as it increased latency of tail and paw withdrawal from 20.3 and 21.0 seconds respectively to 28.9 and 28.4 seconds, as well as eliminated hyperkinesia in 100% of the rats.

Thus, phenylephrine in threshold dose 0.02 mg/kg significantly increased the maximum analgesic effect and decrease effective dose of memantine without developing side hyperkinetic effect.

The potentiated synergism of orally administered combination of memantine with threshold dose 0.02 mg/kg of phenylephrine is highly surprising, comprising a 1.4 fold increase in the maximal analgesic and anti-hyperalgesic effect and a 4 fold decrease in the convention's dose of memantine (from 20 mg/kg to 5 mg/kg). The potentiation of the analgesic and anti-hyperalgesic effect of memantine in this combination cannot be explained by the additional analgesic effect of phenylephrine as the phenylephrine in dose 0.02 mg/kg not has analgesic and anti-hyperalgesic effect.

Combined administration of amitriptyline at a low dose of 0.1 mg/kg and phenylephrine in threshold dose of 0.02 mg/kg practically did not change the sensitivity to pain and hyperalgesia (the latency of the tail and paw withdrawal changed from 4.8 and 4.5 seconds to 6.5 and 6.7 seconds). Combined administration of amitriptyline at a higher dose of 0.5 mg/kg with phenylephrine in threshold dose of 0.02 mg/kg induced a maximal analgesic and anti-hyperalgesic reducing pain sensitivity and hyperalgesia by 4.0 and 4.2 folds, (increase the latency of the tail paw withdrawal respectively from 4.8 and 4.5 seconds to 19.3 and 18.9 seconds), such as amitriptyline alone in high dose of 10 mg/kg. Such a combination did not decrease horizontal activity in the rat. Hence, this combination decreased the effective dose of amitriptyline by 20 times, causing maximal analgesic effect without developing side sedative effect.

Combined administration of amitriptyline at a high dose of 10 mg/kg with phenylephrine in threshold dose of 0.02 mg/kg increased the maximal analgesic and anti-hyperalgesic effect of morphine 1.4 and 1.5 times (increased latency of tail and paw withdrawal from 20.2 and 19.8 seconds respectively to 29.0 and 28.8 seconds), as well as eliminated sedation in 100% of the rats.

Thus, phenylephrine in threshold dose 0.02 mg/kg significantly increased the maximum analgesic effect of amitriptyline in high dose 10 mg/kg without developing side hypokinetic (sedative) effect.

The potentiated synergism of orally administered combination of amitriptyline with threshold dose of phenylephrine (0.02 mg/kg) is highly surprising, because decrease in 20 times effective dose of amitriptyline (from 10 mg/kg to 0.5 mg/kg) and 1.4-1.5 folds increase of the maximal analgesic and anti-hyperalgesic effect of high dose of amitriptyline (10 mg/kg) cannot be explained by the additional analgesic effect of phenylephrine as the phenylephrine in a dose of 0.02 mg/kg not has analgesic and anti-hyperalgesic effect.

Combined administration of dipyrone at a low dose of 0.2 mg/kg and phenylephrine in threshold dose of 0.02 mg/kg practically did not change the sensitivity to pain and hyperalgesia (the latency of the tail and paw withdrawal changed from 4.8 and 4.5 seconds to 6.9 and 6.6 seconds, respectively). Combined administration of dipyrone at a higher dose of 1 mg/kg with phenylephrine in threshold dose of 0.02 mg/kg induced a maximal analgesic and anti-hyperalgesic effect reducing pain sensitivity and hyperalgesia by 4.0 and 4.2 folds, (increase the latency of the tail paw withdrawal respectively from 4.8 and 4.5 seconds respectively to 19.3 and 19.0 seconds), such as dipyrone alone in high dose of 20 mg/kg. Such a combination did not change horizontal activity in the rat. Hence, this combination decreased the effective dose of dipyrone by 20 times, causing maximal analgesic effect without developing side locomotor effect.

Combined administration of dipyrone at a high dose of 20 mg/kg with phenylephrine in threshold dose of 0.02 mg/kg increased the maximal analgesic and anti-hyperalgesic effect of dipyrone by 1.4 and 1.5 times (increased latency of tail and paw withdrawal from 20.6 and 20.1 seconds respectively to 29.4 and 29.2 seconds) without changes of horizontal activity in open field test.

Thus phenylephrine in threshold dose 0.02 mg/kg significantly increased the maximum analgesic effect of dipyrone in high dose 20 mg/kg without developing side locomotor effect.

The potentiated synergism of orally administered combination of dipyrone with threshold dose of phenylephrine (0.02 mg/kg) is highly surprising, because decrease in 20 times effective dose of dipyrone (from 20 mg/kg to 1 mg/kg) and 1.4-1.5 folds increase of the maximal analgesic and anti-hyperalgesic effect of high dose of dipyrone (20 mg/kg). Potentiation cannot be explained by the additional analgesic effect of phenylephrine as the phenylephrine in a dose of 0.02 mg/kg not has analgesic and anti-hyperalgesic effect.

The results of these studies indicate that “double” compositions containing phenylephrine with morphine, memantine, amitriptyline and dipyrone exert analgesic and anti-hyperalgesic effect which is vastly superior to the analgesic and anti-hyperalgesic effect of morphine, memantine, amitriptyline and dipyrone in the conventional dose alone, without developing side sedative and hyperkinetic action and significantly reducing of the conventional doses of morphine, memantine, amitriptyline, dipyrone and phenylephrine in the composition. The above-mentioned compositions cause a potentiation of the analgesic and anti-hyperalgesic action of morphine, amitriptyline, dipyrone and memantine in a safe manner, since they eliminate the side effects of the use of each of the components of the composition.

### Example 11. Potentiation of the Anti-Parkinsonian Effect and Reduction of the Side Effects of Levodopa and Memantine in a Haloperidol Catalepsy Model (Oral Administration)

### Methods.

Anti-parkinsonian effect of drugs was investigated on a model of induced catalepsy by I.M. injection of haloperidol at a dose of 1 mg/kg in Wistar rats (weight 160-170 g). Catalepsy was evaluated by duration of immobilization of rats positioned on a large grid fixed at an angle of 45 degrees 60 minutes after the haloperidol administration. Maximum catalepsy in rats was recorded in the case of their complete immobilization on grid after 180 seconds of observation (Campbell A., et al., 1988, Neuropharmacology, Vol. 27(11), pages 1197-1199; Ossowska K. J., Neural. Transm. Park. Dis. Dement. Sect., 1994, Vol. 8(1-2), pages 39-71; Gmiro V.E. and Serdyuk S.E., Bulletin of Experimental Biology and Medicine, 2007, Vol. 143 (5), pages 554-556).

Levodopa, memantine, phenylephrine, as well as double combinations of phenylephrine with memantine or levodopa were administered orally in a volume of 1.0 ml through a rigid metal probe, 45 minutes before the administration of haloperidol. Control animals received orally 1 ml of distilled water.

Anti-parkinsonian drugs effects were estimated as decrease of the average duration of immobilization of animals treated by test compositions compared to a control (DW).

### Open Field Test.

To evaluate the (hyperkinetic) side effects of tested drugs an “open field” (OF) test was applied. In the OF test, the locomotive activity of rats is determined Animals were placed in the center of square of the illuminated field (1 meter×1 meter) for 3 minutes and the mobility time was recorded in seconds (horizontal activity). The registration of horizontal locomotive activity was performed 40 minutes after administration of the test drug(s), 5 minutes before the administration of haloperidol. To quantify the locomotive activity for each dose of test drug(s) an average horizontal activity was calculated. Hyperkinetic effect of levodopa, phenylephrine, memantine, as well as of combinations phenylephrine with memantine and levodopa was evaluated by an average increase in horizontal activity in the OF test in % compared with measures for rats in the control group, and also as the number of rats with significant increase in horizontal activity (50% and more as compared to control).

Intramuscular (IM) injection of haloperidol at a dose of 1 mg/kg, 60 minutes after injection, results in the control group in immobilization of 158.2±25 seconds (Table 13).

TABLE 13

| Levodopa | Memantine | Phenylephrine | Time on grid | Hyperkinesia | Levodopa | Memantine | Phenylephrine |
|---|---|---|---|---|---|---|---|
| (Rat mg/kg) | (Rat mg/kg) | (Rat mg/kg) | (seconds) | (% of Rats) | (mg, Human Dose) ** | (mg, Human Dose) ** | (mg, Human Dose) ** |
| | | | 158.2* | 0* | | | |
| 1 | | | 147 | 0 | 9.6 | | |
| 3 | | | 125 | 0 | 29 | | |
| 10 | | | 85 | 50 | 96.7 | | |
| 20 | | | 35 | 100 | 193.5 | | |
| | | 0.1 | 137 | 0 | | | 0.9 |
| | | 0.3 | 98 | 0 | | | 2.9 |
| | | 1 | 78 | 0 | | | 9.6 |
| | 1 | | 139 | 0 | | 9.6 | |
| | 3 | | 108 | 0 | | 29 | |
| | 10 | | 65 | 50 | | 96.7 | |
| | 20 | | 34 | 100 | | 193.5 | |
| | 0.5 | 0.1 | 133 | 0 | | | |
| | 1 | 0.1 | 79 | 0 | | | |
| | 3 | 0.1 | 35 | 0 | | | |
| | 10 | 0.1 | 12 | 0 | | | |
| 1.5 | | 0.1 | 75 | 0 | 14.5 | | |
| 3.0 | | 0.1 | 37 | 0 | 29 | | |
| 10 | | 0.1 | 21 | 0 | 96.7 | | |
| 10 | | 0.3 | 12 | 0 | 96.7 | | |

*N.C.—Negative control (distilled water).
** Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).

### Dual-Therapy.

Combined administration of levodopa in low dose of 1.5 mg/kg and phenylephrine at a threshold dose (0.1 mg/kg, which was ineffective independently) caused a mild but significant anti-parkinsonian effect, as it reduced haloperidol catalepsy by a factor of 2.1 (reduces the duration of immobilization on the grid from 158 to 75 seconds). Combined levodopa administration in a low dose of 3 mg/kg with phenylephrine at a threshold dose of 0.1 mg/kg caused a significant anti-parkinsonian effect (reduced the duration of immobilization on a grid by a factor of 4.3, from 158 to 37 seconds), similar to levodopa in the high dose of 20 mg/kg alone. It is important to note that such a combination has not increased the horizontal activity in rats. Therefore, a threshold dose of phenylephrine (0.1 mg/kg) reduces the effective dose of levodopa by about 7 times, achieving the maximal effect without developing side effects.

Combined levodopa administration in doses of 10 mg/kg with phenylephrine at a threshold dose of 0.1 mg/kg and low conventional dose 0.3 mg/kg had a maximal anti-parkinsonian effect (reduced the duration of immobilization on the grid from 158 to 21 and 12 seconds, respectively), which is 1.8 and 3.0 times better than levodopa in the high dose of 20 mg/kg alone. It is important to note that such combinations had also not increased the horizontal activity in rats. Therefore, a dose of phenylephrine (0.1-0.3 mg/kg) reduced the effective dose of levodopa by 2 times and increase maximal effect of levodopa without developing side effects.

The potentiated synergism of orally administered combination of levodopa with threshold 0.1 mg/kg and low conventional dose of phenylephrine (0.3 mg/kg) is highly surprising, comprising a 1.8-3.0 fold increase in the maximal antiparkinsonian effect and a 2 fold decrease in the convention's dose of levodopa (from 20 mg/kg to 10 mg/kg). The potentiation of the antiparkinsonian effect of levodopa in this combination cannot be explained by the additional antiparkinsonian effect of phenylephrine as the phenylephrine in dose 0.1-0.3 mg/kg has very weak antiparkinsonian effect.

The results of these studies indicate that “double” compositions containing levodopa and phenylephrine exert anti-parkinsonian effect which is vastly superior to the anti-parkinsonian effect of levodopa in the conventional dose alone, without developing hyperkinetic side effect and significantly reducing the dosage of conventional levodopa and phenylephrine in the composition. The above-mentioned compositions cause a potentiation of the anti-parkinsonian action of levodopa in a safe manner, since they eliminate the side effects of the use of each of the components of the composition.

Combined administration of memantine in low dose of 0.5 mg/kg and phenylephrine at a threshold dose (0.1 mg/kg, which was ineffective independently) caused a not significant anti-parkinsonian effect, as it reduced haloperidol catalepsy by a factor of 1.2 (reduces the duration of immobilization on the grid from 158 to 133 seconds)

Combined administration of memantine in low dose of 1.0 mg/kg and phenylephrine at a threshold dose (0.1 mg/kg, caused a mild but significant anti-parkinsonian effect, as it reduced haloperidol catalepsy by a factor of 2.0 (reduces the duration of immobilization on the grid from 158 to 79 seconds). Combined memantine administration in a low dose of 3 mg/kg with phenylephrine at a threshold dose of 0.1 mg/kg caused a significant anti-parkinsonian effect (reduced the duration of immobilization on a grid by a factor of 4.5, from 158 to 35 seconds), similar to memantine in the high dose of 20 mg/kg alone. It is important to note that such a combination has not increased the horizontal activity in rats. Therefore, a threshold dose of phenylephrine (0.1 mg/kg) reduces the effective dose of memantine by about 7 times, achieving the maximal effect without developing side effects.

Combined memantine administration in doses of 10 mg/kg with phenylephrine at a threshold dose of 0.1 mg/kg had a maximal anti-parkinsonian effect (reduced the duration of immobilization on the grid from 158 to 12 seconds, respectively), which is 2.9 times better than memantine in the high dose of 20 mg/kg alone. It is important to note that such combinations had also not increased the horizontal activity in rats. Therefore, a dose of phenylephrine 0.1 mg/kg reduced the effective dose of memantine by 2 times and increase maximal effect of memantine without developing side effects.

The potentiated synergism of orally administered combination of memantine with threshold 0.1 mg/kg of phenylephrine is highly surprising, comprising a 2.8 fold increase in the maximal antiparkinsonian effect and a 2 fold decrease in the convention's dose of memantine (from 20 mg/kg to 10 mg/kg). The potentiation of the antiparkinsonian effect of memantine in this combination cannot be explained by the additional antiparkinsonian effect of phenylephrine as the phenylephrine in dose 0.1 mg/kg has very weak antiparkinsonian effect.

The results of these studies indicate that “double” compositions containing phenylephrine with levodopa and memantine exert anti-parkinsonian effect which is vastly superior to the anti-parkinsonian effect of levodopa and memantine in the conventional dose alone, without developing hyperkinetic side effect and significantly reducing the dosage of conventional levodopa, memantine and phenylephrine in the composition. The above-mentioned compositions cause a potentiation of the anti-parkinsonian action of levodopa and memantine in a safe manner, since they eliminate the side effects of the use of each of the components of the composition.

### Example 12. Potentiation of the Anti-Parkinsonian and Neuro-Protective Effects, and Elimination of Side Effects, of Levodopa and Memantine in Rotenone-Induced Parkinson Disease (PD) and Progressive Supranuclear Palsy (PSP) in Rats

### Administration of Rotenone.

Rotenone was dissolved in mixture of DMSO and Miglyol 812N at a ratio of 2:98 at concentration of 2.3 mg/ml. Preparation: 230 mg sample of rotenone in vials was dissolved in 2 ml of DMSO. The solution was poured into a jar with 100 ml. Vials are rinsed twice with 5 ml of Miglyol, washings attached to the main solution and stirred. To this solution was added 88 ml of Miglyol and mixed again. The resulting solution was divided into 3 doses. The solution was stored at −10° C. Each animal was administered 0.2 ml of a solution of rotenone: a dose of 0.46 mg per animal weighing 200±20 g, i.e. 2.3 mg/kg. The solution of rotenone was injected IP one time a day for 19 days.

Anti-parkinsonian effect of therapy in rats with rotenone-induced PD was estimated according to elimination of extrapyramidal disorders (catalepsy) and oligokinesia in the “open field” test. The neuro-protective effect of therapy was evaluated by the elimination of ataxia limb dystonia and mortality in rats with rotenone-induced PSP. Hyperkinetic side action (hyper-locomotion) was evaluated by increased horizontal and vertical activity in the open field at the 10th and 18th day of the experiment, calculated in % compared to the first day of the experiment.

Phenylephrine 0.3 mg/kg, levodopa (10 and 20 mg/kg), memantine 2.5 and 5 mg/kg, a combination of levodopa 10 mg/kg+phenylephrine 0.3 mg/kg, a combination of levodopa 20 mg/kg+phenylephrine 0.3 mg/kg, combination of memantine 1 mg/kg+phenylephrine 0.3 mg/kg, combination of memantine 2.5 mg/kg+phenylephrine 0.3 mg/kg, combination of memantine 5 mg/kg+phenylephrine 0.3 mg/kg, and DW (1.0 ml, control) were orally administered using rigid metal probe in volumes of 1 ml daily for 19 days, 45 minutes before administration of rotenone. The number of animals in the control and experimental groups ranged from 6 to 8.

Levodopa was administered in combination with benserazide (peripheral dopa-decarboxylase inhibitor) at a ratio of 4:1 to reduce the side effects of levodopa, associated with the stimulation of peripheral dopamine receptors (Alam M. et al., Behav. Brain Res., 2004, Vol. 153 (2), pages 439-446). Levodopa with benserazide were dissolved in 1 ml of DW and administered orally in the volume of 1 ml. Combination of levodopa with phenylephrine, of levodopa and of benserazide solution (0.5 ml) was mixed with 0.5 ml of phenylephrine solution. Combination of levodopa with memantine and phenylephrine of levodopa and of benserazide solution (0.5 ml) was mixed with 0.5 ml of phenylephrine+memantine solution. The mixture was administered orally in a volume of 1 ml. Anti-parkinsonian effect in the experimental groups of rats with rotenone has been evaluated by eliminating catalepsy and oligokinesia.

Catalepsy in rats is determined by the time of immobilization of the animal, placed on a large grid predisposed at an angle of 45°. Maximum catalepsy in rats is recorded in the case of their complete immobilization on grid at 120 seconds for observation. The value of catalepsy is evaluated in points: 3 points—immobilization duration from 80 seconds to 120, 2 points—immobilization duration from 40 to 70, 1 point—immobilization duration from 20 to 35, 0 points—the immobilization of less than 20 seconds. Catalepsy was followed in rats daily, 30 minutes before administration of rotenone and 180 minutes after administration of rotenone. For each experimental group, on a daily basis, throughout the period of observation number of rats with severe catalepsy (2-3 points) was determined in % of the total number of rats in group. To assess the anti-cataleptic effects of therapy, on 12th and 19th day of experiment, reduction in the number of rats with severe catalepsy (2-3 points) in % was determined compared to control treated with distilled water.

Locomotive activity of rats was tested in the “open field” test. Animals were placed in the center of square of the illuminated field (1 meter×1 meter) and during 3 minutes the distance of movement (horizontal activity) and the number of rearing (vertical activity) was recorded. Registration of horizontal and vertical locomotive activity was carried out in first day for rats with high selection locomotive activity (total walking time with not less than 12 seconds and the number of vertical uprights at least 4), 60 minutes before the first administration of rotenone. To identify the rotenone-induced oligokynesia in each experimental group the locomotive activity in the open field test was examined again 2 hours after the administration of rotenone on days 12 and 19 of the experiment.

Quantification of oligokynesia in points: 0 points—the highest horizontal activity (walking total time more than 12 seconds) and high vertical activity (number of vertical posts) more than 3; 1 point—reducing the horizontal activity (total walking time 7-11 seconds) and a decrease in vertical activity (the number of vertical columns 1-3); 2 points—a significant reduction in horizontal activity (total walking time 2-6 seconds) and the absence of vertical activity (the number of vertical columns 0-1); 3 points—the absence of horizontal activity (total walking time 0-1 seconds) and the absence of vertical activity (number of vertical columns 0). In each experimental group 120 minutes after administration of rotenone on days 12 and 19 of experiment the number of rats with severe oligokinesia (2-3 points) in % of the total number of rats in group was determined.

The neuro-protective effect in the experimental groups of rats with rotenone-induced PSP was evaluated due the elimination of ataxia, limb dystonia and mortality caused by cerebellar neurotoxic action of rotenone (J. Neurochem., 2005, Vol. 95(4):930-9).

Ataxia and limb dystonia is typical incoordination neurotoxic effect of rotenone manifested in uncoordinated movements and hyper-locomotion, replaced by hypokinesia and stereotypes, and in severe cases a complete violation of antigravity reflexes, falling on its side dystonic posture and akinesia. Assessment of the severity of ataxia and limb dystonia in scores was performed using the following scale: (+) uncoordinated movement, hyperkinesia; (++) significant incoordination, hypokinesia, stereotypes, falling on its side; frequent spontaneous dystonic postures (+++) complete violation of antigravity reflexes, akinesia, sustained dystonic posture (J. Neurosci., 2014, 27; 34 (35):11723-32)

Ataxia and limb dystonia registration was performed twice daily, 30 minutes before administration of rotenone and 180 minutes after administration of rotenone. For each experimental group on a daily basis, throughout the whole period of observation number of rats with severe ataxia and limb dystonia (++−+++) and mortality in % of the total number of rats in group was determined. The neuro-protective effect of therapy (effects on rate of ataxia, limb dystonia and lethality) in rats with PSP was determined at days 12 and 19 of the experiment as reducing the number of rats with severe ataxia limb dystonia and mortality in % compared to control. Side hyperkinetic effects were evaluated in the “open field” test. In this test the locomotive activity in rats is determined. Animals were placed in the center of square of the illuminated field (1 meter×1 meter) for 3 min total time recorded in seconds distance (horizontal activity) and the number of rearing (vertical activity). Registration of horizontal and vertical locomotive activity in OF assay was performed on the 10th and 18th day of the experiment, within 40 minutes after administration of the substance and 5 minutes before administration of rotenone. Side hyperkinetic effects was evaluated by the number of rats with a significant (at least 50% compared with the first day of the experiment) increase in horizontal and vertical activity (hyperkinesia) as a % of the total number of rats in group.

TABLE 14
Potentiation of the anti-parkinsonian and neuro-protective effects, and elimination
of side effects of levodopa¹ and memantine in rotenone-induced PD and PSP in rats.

| | | | | | | | Severe ataxia | | | | | | |
|---|---|---|---|---|---|---|---|---|---|---|---|---|---|
| | | | | | | | sustained dystonic | | | | | | |
| Doses for rat | | | Severe | | Severe | | postures and lethality | | | | Doses for human ² | | |
| (mg/kg) | | | catalepsy | | oligokinesia | | (% of group)*** | | Hyperkmesia**** | | (mg) | | |
| | Phenyl- | Mem- | (% of group) | | (% of group) | | [lethality] | | (% of group) | | | Phenyl- | Mem- |
| Levodopa | ephrine | antine | Day 12 | Day 19 | Day 12 | Day 19 | Day 12 | Day 19 | Day 10 | Day 18 | Levodopa | ephrine | antine |
| | | | 42³ | 58³ | 100³ | 100³ | 14[0]³ | 42[14]³ | 0³ | 0³ | | | |
| | | 2.5 | 33 | 50 | 83 | 100 | 17[17] | 34[17] | 0 | 0 | | | 24 |
| | | 5 | 17 | 17 | 50 | 50 | 0[17] | 17[17] | 40 | 60 | | | 48 |
| | 0.3 | | 28 | 42 | 86 | 100 | 14[0] | 28[14] | 0 | 0 | | 2.9 | |
| 10 | | | 28 | 42 | 86 | 100 | 14[0] | 42[14] | 0 | 0 | 96.7 | | |
| 20 | | | 12 | 24 | 50 | 63 | 0[0] | 24[0] | 88 | 75 | 193.5 | | |
| | 0.3 | 1 | 50 | 50 | 83 | 100 | 17[17] | 34[17] | 0 | 0 | | | |
| | 0.3 | 2.5 | 17 | 17 | 33 | 50 | 0[17] | 17[17] | 0 | 0 | | | 9.6 |
| | 0.3 | 5.0 | 0 | 0 | 17 | 34 | 0 | 0[17] | 0 | 0 | | | 24 |
| 10 | 0.3 | | 0 | 12 | 24 | 24 | 0 | 0 | 0 | 0 | 96.7 | 2.9 | 48 |
| 20 | 0.3 | | 0 | 0 | 12 | 12 | 0 | 0 | 12 | 0 | 193.5 | 2.9 | |

¹Oral administration 45 minutes before the administration of rotenone 2.3 mg/kg in duration 19 days.
² Human Dose—Absolute dose for a 60 kg Human, calculated according to FDA guidelines (Guidance for Industry, “Estimating the Maximum Safe Starting Dose in Initial Clinical Trials for Therapeutics in Adult Healthy Volunteers”, July 2005, page 7).
³N.C.—Negative control (distilled water).
*The immobilization time of a rat on an inclined grid above 40 seconds.
**Oligokinesia ++-+++ in OF test.
***Ataxia and limb dystonia ++-+++.
****Increased locomotive activity in OF test more than 50% compared to the first day of experiment.

### Dual-Therapy.

A combination of levodopa (10 mg/kg) with phenylephrine (0.3 mg/kg) upon chronic oral administration on days 12 and 19 of experiment had reduced the number of rats with severe oligokinesia by 76% compared to control (from 100% to 24%) indicating a very significant reduction of oligokinesia in rats with rotenone-induced PD. This combination on days 12 and 19 of the experiment reduced the number of rats with severe catalepsy with maximum efficiency to 42% and 46% respectively compared to control (from 42% to 0% and from 58% to 12%). A combination of levodopa 10 mg/kg and phenylephrine 0.3 mg/kg significant (on 30%) reduction ataxia, limbic dystonia and mortality at days 19 of experiment from 42% to 12%. A combination of levodopa in a dose of 10 mg/kg and phenylephrine in a dose of 0.3 mg/kg did not cause hyperkinesia in rats in the OF test on the 10th and 18th day of experiment. Thus, the combination of levodopa in a conventional low dose (10 mg/kg) and phenylephrine in the low convention dose of 0.3 mg/kg eliminated catalepsy and significant decrease oligokinesia in rats with rotenone-induced PD and very significant reduction ataxia, limbic dystonia and mortality in rats with rotenone-induced PSP. The advantage of the aforementioned combination is a complete absence of hyperkinetic side effect in rats.

Chronic oral administration of combination of levodopa in a dose of 20 mg/kg with phenylephrine in a dose of 0.3 mg/kg reduced the number of rats with severe oligokinesia by 88% compared to control (from 100% to 12%). This indicates that this combination eliminated oligokinesia in rats with rotenone-induced PD. This combination completely eliminated heavy catalepsy in rats with rotenone-induced PD (decrease from 42% to 0% and from 58% to 0%). A combination of levodopa in a dose of 20 mg/kg and phenylephrine in a dose of 0.3 mg/kg completely eliminated ataxia limbic dystonia and mortality on days 12 and 19 of the experiment. A combination of levodopa in a dose of 20 mg/kg and phenylephrine in a dose of 0.3 mg/kg not increase of locomotive activity of the rats on 18th day of the experiment. Therefore, chronic orally administered combination of levodopa in a high conventional dose of 20 mg/kg and phenylephrine in a conventional low dose 0.3 mg/kg to rats efficiently eliminated the extrapyramidal and motor disorders in rats with rotenone-induced PD and completely eliminated ataxia, limbic dystonia and mortality in rats with rotenone-induced PSP, and virtually eliminates the hyperkinesia induced by a high dose of levodopa. Thus, oral administration of levodopa in the low conventional dose 10 mg/kg and in the high conventional dose of 20 mg/kg with phenylephrine in a low dose of 0.3 mg/kg to rats increases the anti-parkinsonian and neuro-protective effect that is achieved by levodopa in a dose of 20 mg/kg by 50-100% without developing side hyperkinetic effects. The anti-parkinsonian and neuro-protective activity of the combination of levodopa in a conventional lowest dose of 10 mg/kg with phenylephrine in a conventional lowest dose of 0.3 mg/kg is highly surprising, as it far exceeds the anti-parkinsonian and neuro-protective activity of each one of the components of the combination alone, as well as it surpasses (by 1.5-2 times) the anti-parkinsonian and neuro-protective activity of levodopa in a double dose of 20 mg/kg. Therefore, phenylephrine that is not effective alone in a dose of 0.3 mg/kg synergistically potentiated the anti-parkinsonian and neuro-protective effects of levodopa in a low dose of 10 mg/kg that had low efficacy while being used alone, to the maximal level, which cannot be explained in view of the independent use of levodopa in the high dose of 20 mg/kg alone. However, phenylephrine did not enhance the hyperkinetic side effect of levodopa at dose of 10 mg/kg.

It is important to notice the relatively small difference in neuro-protective and anti-parkinsonian activity of the combination of phenylephrine with levodopa at a dose of 20 mg/kg and 10 mg/kg. This implies that much less phenylephrine enhanced the therapeutic effects of levodopa in the high dose of 20 mg/kg, but effectively eliminated the hyperkinetic side effects of levodopa in high dose of 20 mg/kg. According to the results of the experiments, the combination of levodopa with phenylephrine in conventional low doses can be offered for the safe treatment of severe Parkinson's disease and supranuclear progressive palsy, resistant to the action of levodopa, as well as for the treatment of Parkinson's disease and PSP in patients who cannot tolerate levodopa.

Chronic oral administration of combination of memantine in a low dose 1 mg with phenylephrine in a dose of 0.3 mg/kg caused not significant antiparkinsonian and neuroprotective effect in tats with rotenone parkinsonism and PST. A combination of memantine in low dose 2.5 mg/kg with 0.3 mg/kg phenylephrine upon chronic oral administration on days 12 and 19 of experiment had reduced the number of rats with severe oligokinesia by 67% and 50% compared to control (from 100% to 33% and 50%) indicating a significant reduction of oligokinesia in rats with rotenone-induced PD. This combination on days 12 and 19 of the experiment reduced the number of rats with severe catalepsy with maximum efficiency to 29% and 41% respectively compared to control (from 42% to 17% and from 58% to 17%). A combination of 2.5 mg/kg memantine and 0.3 mg/kg phenylephrine significantly caused a 25% reduction in ataxia, limbic dystonia and mortality at days 19 of the experiment, from 42% to 17%. A combination of memantine in a dose of 2.5 mg/kg and phenylephrine in a dose of 0.3 mg/kg did not cause hyperkinesia in rats in the OF test on the 10th and 18th day of experiment. Thus, the combination of memantine in a conventional low dose 2.5 mg/kg) and phenylephrine in the low convention dose of 0.3 mg/kg caused submaximal antiparkinsonian and neuroprotective effect compare with efficacy memantine in dose 5 mg/kg alone. The advantage of the aforementioned combination is a complete absence of hyperkinetic side effect in rats.

Chronic oral administration of combination of memantine in a conventional dose 5 mg with phenylephrine in a low dose of 0.3 mg/kg reduced the number of rats with severe oligokinesia on days 12 and 19 of the experiment by 83% and 66% compared to control (from 100% to 17 and 34%). This indicates that this combination very significant decreased oligokinesia in rats with rotenone-induced PD. This combination completely eliminated heavy catalepsy in rats with rotenone-induced PD (decrease from 42% to 0% and from 58% to 0%). A combination of memantine in a dose of 5 mg/kg and phenylephrine in a dose of 0.3 mg/kg fully eliminated ataxia, limbic dystonia and mortality on days 12 and 19 of the experiment. A combination of memantine in a dose of 5 mg/kg and phenylephrine in a dose of 0.3 mg/kg not increase of locomotive activity of the rats on 18th day of the experiment. Therefore, chronic orally administered combination of memantine in a conventional dose of 5 mg/kg and phenylephrine in a conventional low dose 0.3 mg/kg to rats efficiently eliminated the extrapyramidal and motor disorders in rats with rotenone-induced PD and completely eliminated ataxia, limbic dystonia and mortality in rats with rotenone-induced PSP, and virtually eliminates the hyperkinesia induced by a conventional dose of memantine. Thus, oral administration of memantine in the conventional dose 5 mg/kg and with phenylephrine in a low dose of 0.3 mg/kg to rats increases the anti-parkinsonian and neuro-protective effect that is achieved by memantine in a dose of 5 mg/kg by 50-70% without developing side hyperkinetic effects. The anti-parkinsonian and neuro-protective activity of the combination of memantine in a low dose 2.5 mg/kg and conventional dose of 5 mg/kg with phenylephrine in a conventional lowest dose of 0.3 mg/kg is highly surprising, as it far exceeds the anti-parkinsonian and neuro-protective activity of each one of the components of the combination alone. Therefore, phenylephrine that is not effective alone in a dose of 0.3 mg/kg synergistically potentiated the anti-parkinsonian and neuro-protective effects of memantine in a low dose of 2.5 mg/kg and conventional dose 5 mg/kg to the maximal level, which cannot be explained in view of the independent use of memantine in this dose alone. However, phenylephrine did not enhance the hyperkinetic side effect of memantine in low 2.5 mg/kg but effectively eliminated the hyperkinetic side effects of memantine in higher dose of 5 mg/kg.

According to the results of the experiments, the combination of memantine with phenylephrine in conventional low doses can be offered for the safe treatment of severe Parkinson's disease and supranuclear progressive palsy, resistant to the action of levodopa, as well as for the treatment of Parkinson's disease and PSP in patients who cannot tolerate levodopa.

It is important to note that the scope of the invention is not construed as being limited by the illustrative embodiments set forth herein. Other variations are possible within the scope of the present invention as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1-48. (canceled)

49. A method of potentiating the activity or reducing at least one side effect of a CNS drug selected from the group consisting of an anti-parkinsonian drug and an analgesic drug in a subject in need, comprising systemically administering to the subject:
a) at least one first CNS drug, selected from the group consisting of an anti-parkinsonian drug and an analgesic drug;
b) at least one second CNS drug, which is an NMDA receptor antagonist; and
c) at least one peripheral adrenergic receptor agonist.

50. The method of claim 49, wherein the anti-parkinsonian drug is selected from the group consisting of:
a) L-3,4-dihydroxyphenylalanine (levodopa);
b) a dopamine agonist selected from the group consisting of bromocriptine, cabergoline, pergolide, pramipexole, ropinirole, piribedil, apomorphine, rigotine, quinagolide, fenoldopam, and lisuride; and
c) a monoamine oxidase B (MAOB) inhibitor selected from the group consisting of selegiline, desmethylselegiline, pargyline, rasagiline, lazabemide, milacemide, mofegiline, D-deprenyl and ladostigil.

51. The method of claim 49, wherein the analgesic drug is selected from the group consisting of morphine, amitriptyline, dipyrone, fentanyl, promedolum, omnoponum, oxycodone, hydrocodone, hydromorphone, hydrocodone bitartrate, and buprenorphine.

52. The method of claim 49, wherein the NMDA receptor antagonist is selected from the group of memantine, amantadine, dextromethorphan and ketamine.

53. The method of claim 49, wherein the peripheral adrenergic receptor agonist is selected from the group consisting of phenylephrine, epinephrine, midodrine and pseudoephedrine.

54. The method claim 49, wherein first CNS drug is levodopa, morphine, amitriptyline or dipyrone, the NMDA receptor antagonist is memantine, and the peripheral adrenergic receptor agonist is phenylephrine or epinephrine.

55. The method of claim 49, wherein the side effect is selected from the group consisting hyperkinesia, sedation, hyperalgesia, catalepsy, dyskinesia, and addiction.

56. The method of claim 50, comprising systemically administering to the subject 5-200 mg levodopa.

57. The method of claim 51, comprising systemically administering to the subject 0.1-50 mg morphine.

58. The method of claim 51, comprising systemically administering to the subject 0.5-20 mg amitriptyline.

59. The method of claim 51, comprising systemically administering to the subject 0.5-40 mg dipyrone.

60. The method of claim 52, comprising systemically administering to the subject 5-30 mg memantine.

61. The method of claim 53, comprising systemically administering to the subject 0.1-3 mg phenylephrine.

62. The method of claim 53, comprising systemically administering to the subject 0.05-0.1 mg epinephrine.

63. The method of claim 49, comprising systemically administering to the subject the first CNS drug, the second CNS drug and the peripheral adrenergic receptor agonist in a molar ratio of 0.2-1000:1-300:1, respectively.

64. The method of claim 49, for treating Parkinson Disease (PD) or Progressive Supranuclear Palsy (PSP) or Parkinsonism syndrome.

65. The method of claim 49, for treating pain or hyperalgesia.

66. A pharmaceutical composition, comprising:
a) at least one first CNS drug, selected from the group consisting of an anti-parkinsonian drug and an analgesic drug;
b) at least one second CNS drug, which is an NMDA receptor antagonist; and
c) at least one peripheral adrenergic receptor agonist.

67. A method of potentiating the activity or reducing at least one side effect of a CNS drug selected from the group consisting of an anti-parkinsonian drug and an analgesic drug in a subject in need, comprising systemically administering to the subject:
a) at least one CNS drug, wherein the CNS drug is levodopa; and
b) at least one peripheral adrenergic receptor agonist, wherein the peripheral adrenergic receptor agonist is phenylephrine or epinephrine;
wherein the molar ratio of the CNS drug and peripheral adrenergic receptor agonist is in the range of 1-2000:1, respectively.

68. The method of claim 67, for treating Parkinson Disease (PD) or Progressive Supranuclear Palsy (PSP) or Parkinsonism syndrome.
